(19) United States
(12) Patent Application Publication
(10) Pub. No.: US 2015/0337376 A1
(43) Pub. Date: Nov. 26, 2015

(54) Core Transcriptional Circuitry in Human Cells and Methods of Use Thereof

(71) Applicant: Whitehead Institute for Biomedical Research, Cambridge, MA (US)

(72) Inventors: Violaine Saint-Andre, Cambridge, MA (US); Brian J. Abraham, Cambridge, MA (US); Zi Peng Fan, Waltham, MA (US); Tong Ihn Lee, Somerville, MA (US); Richard A. Young, Boston, MA (US)

(21) Appl. No.: 14/663,056

(22) Filed: Mar. 19, 2015

Related U.S. Application Data
(60) Provisional application No. 61/955,764, filed on Mar. 19, 2014.

(51) Int. Cl. C12Q 1/68

(52) U.S. Cl. CPC C12Q 1/6883; C12Q 2600/156; C12Q 2600/136

(57) ABSTRACT

Disclosed herein are methods for identifying the core regulatory circuitry or cell identity program of a cell or tissue, and related methods of diagnoses, screening, and treatment involving the core regulatory circuitry and/or cell identity programs identified using the methods.

### RELATED APPLICATIONS

This application claims the benefit of U.S. Provisional 61/955,764, filed Mar. 19, 2014. The entire teachings of the above application(s) are incorporated herein by reference.

### GOVERNMENT SUPPORT

This invention was made with government support under RO1-HG002668 awarded by the National Institutes of Health. The government has certain rights in the invention.

### BACKGROUND OF THE INVENTION

The molecular pathways for cellular processes such as metabolism, energy production, and signal transduction have been described in some detail. In contrast, the transcriptional circuitries that control the gene expression programs that define cell identity have yet to be mapped in most cells. For such mapping, it is essential to identify the set of key transcription factors that are responsible for control of cell identity and to determine how they function together to regulate cell-type-specific gene expression programs.

### SUMMARY OF THE INVENTION

In some aspects, the disclosure provides a method of identifying the core regulatory circuitry of a cell or tissue, comprising: a) identifying a group of transcription factor encoding genes in a cell or tissue which are associated with a super-enhancer; b) determining which transcription factor encoding genes identified in a) comprise autoregulated transcription factor encoding genes, wherein a transcription factor encoding gene identified in a) comprises an autoregulated transcription factor encoding gene if the transcription factor encoded by the transcription factor encoding gene is predicted to bind to the super-enhancer associated with the transcription factor encoding gene; and c) identifying the core regulatory circuitry of the cell or tissue, wherein the core regulatory circuitry of the cell or tissue comprises autoregulated transcription factor encoding genes identified in b) which form an interconnected autoregulatory loop, wherein the autoregulated transcription factor encoding genes identified in b) form an interconnected autoregulatory loop if each transcription factor encoded by an autoregulated transcription factor encoding gene identified in b) is predicted to bind to the super-enhancer associated with each of the other autoregulated transcription factor encoding genes identified in b).

In some embodiments, the core regulatory circuitry comprises the autoregulated transcription factors forming the interconnected autoregulatory loop, the transcription factors encoded by the autoregulated transcription factor encoding genes, a super-enhancers associated with the autoregulated transcription factor encoding genes, or a component of the super-enhancer.

In some embodiments, the method further includes d) determining at least one target of at least one transcription factor encoded by at least one autoregulated transcription factor encoding gene. In some embodiments, the at least one target of the at least one transcription factor encoded by the at least one autoregulated transcription factor encoding gene comprises a gene which encodes a reprogramming factor or a cell identity gene. In some embodiments, the transcription factor encoded by the transcription factor encoding gene is predicted to bind to the super-enhancer associated with transcription factor encoding gene if the super-enhancer associated with the transcription factor encoding gene comprises at least one DNA sequence motif predicted for the transcription factor encoded by the transcription factor encoding gene. In some embodiments, each transcription factor encoded by the autoregulated transcription factor encoding gene is predicted to bind to the super-enhancer associated with each of the other autoregulated transcription factor encoding genes if the super-enhancers associated with each of the other autoregulated transcription factor encoding genes comprise at least one DNA sequence motif predicted for each of the transcription factors encoded by each of the other autoregulated transcription factor encoding genes.

In some embodiments, the at least one DNA sequence motif is located between 500 bp upstream and 500 bp downstream of the super-enhancer associated with the transcription factor encoding gene.

In some embodiments, the cell comprises a) a blood cell selected from the group consisting of a CD14+ monocyte, a CD56+ monocyte, a CD4+ T cell, a CD3+ T cell, a CD4+ primary T cell, a CD4+ memory T cell, a CD4+ naïve T cell, a CD4+ CD127+ T cell, a CD8+ primary T cell, a CD8+ memory T cell, a CD8+ naïve T cell, a CD19+ B cell, a CD20+ B cell, a CD34+ HSC cell; b) a brain cell selected from the group consisting of astrocytes, glial cells, an neurons; c) a fibroblast selected from the group consisting of dermal fibroblast and fibroblast; d) skeletal myoblasts; e) a colon crypt, f) an embryonic stem cell; g) a hepatocyte; h) a tumor cell; i) a keratinocyte; j) a macrophage; k) lymphocytes; l) regulatory T (Tregs); m) NK cells; n) pancreatic beta cells; o) cardiac muscle cells; p) never cells; and q) chondrocytes.

In some embodiments, the tissue comprises a) brain tissue selected from the group consisting of brain hippocampus, brain inferior temporal lobe, brain angular gyrus, and brain mid frontal lobe; b) internal tissue selected from the group consisting of spleen, bladder, mammary epithelium, adipose, ovarian, adrenal gland, pancreatic, and lung; d) thymus; e) muscle tissue selected from the group consisting of skeletal muscle, psoas muscle, duodenum smooth muscle, and stomach smooth muscle; f) heart tissue selected from the group consisting of right ventricle, aorta, left ventricle, and right atrium; g) digestive tissue selected from the group consisting of esophagus, gastric, sigmoid colon, and small intestine; and h) tumor tissue.

In some aspects, the disclosure provides a method of identifying the cell identity program of a cell or tissue, comprising a) identifying the core regulatory circuitry of a cell or tissue of interest, wherein the core regulatory circuitry of the cell or tissue of interest comprises at least one autoregulated transcription factor encoding gene associated with a super-enhancer in the cell or tissue of interest, at least one transcription factor encoded by the at least one autoregulated transcription factor encoding gene, at least one super-enhancer associated with the at least one autoregulated transcription factor encoding gene, and optionally at least one component of the super-enhancer; and b) identifying the cell identity program of the cell or tissue, wherein the cell identity program of the cell or tissue comprises the core regulatory circuitry identified in a) and at least one target of the at least one transcription factor encoded by the at least one autoregulated transcription factor encoding gene in the core regulatory circuitry.

In some embodiments, the at least one target comprises a gene comprising at least one enhancer element predicted to be bound by the at least one transcription factor. In some embodiments, the at least one enhancer element predicted to be bound by the at least one transcription factor comprises a DNA sequence motif associated with a super-enhancer.

In some aspects, the disclosure provides a method of modulating the identity of a cell, comprising modulating at least one component of a cell identity program of the cell. In some embodiments, the at least one component of the cell identity program in the cell comprises the core regulatory circuitry of the cell or at least one target modulated by the at least one component of the core regulatory circuitry of the cell. In some embodiments, the modulating the at least one component of the cell identity program in the cell comprises contacting the cell with an agent that modulates at least one component of the cell identity program of the cell.

In some embodiments, the cell comprises a cell listed in Table 2 and the at least one component of the cell identity program comprises at least one component listed in Table 2 selected from the group consisting of (i) at least one gene encoding a master transcription factor, (ii) the master transcription factor encoded by the at least one gene, (iii) a target of the master transcription factor, and (iv) at least one super-enhancer associated with any of (i)-(iii), or at least one component of the super-enhancer.

In some embodiments, the method further includes (i) modulating at least two components of the cell identity program in the cell, (ii) modulating at least three components of the cell identity program in the cell, (iii) modulating at least four components of the cell identity program in the cell, or (iv) modulating at least five components of the cell identity program in the cell. In some embodiments, the method further includes (i) modulating at least one component of the core regulatory circuitry in the cell and at least one target of a master transcription factor in the core regulatory circuitry; (ii) modulating at least two components of the core regulatory circuitry in the cell and at least two targets of a master transcription factor in the core regulatory circuitry; (iii) modulating at least three components of the core regulatory circuitry in the cell and at least three targets of a master transcription factor in the core regulatory circuitry; (iv) modulating at least four components of the core regulatory circuitry in the cell and at least four targets of a master transcription factor in the core regulatory circuitry; and (v) modulating at least five components of the core regulatory circuitry in the cell and at least five targets of a master transcription factor in the core regulatory circuitry of the cell.

In some aspects, the disclosure provides a method of diagnosing a cell identity program-related disorder comprising determining whether the cell identity program of the cell or tissue is enriched for disease-associated variations. In some embodiments, the determining comprises: a) obtaining a sample comprising a cell or tissue of interest; and b) detecting the presence of disease-associated variations in components of the cell identity program of the cell or tissue of interest, wherein the cell identity program of the cell or tissue is enriched for disease-associated variations if at least two disease-associated variations are detected in the components of the cell identity program of the cell or tissue of interest.

In some embodiments, the cell identity program of the cell or tissue is enriched for disease-associated variations if (i) at least three; (ii) at least four; (iii) at least five; (iv) or at least six disease associated variations are detected in the components of the cell identity program of the cell or tissue of interest. In some embodiments, the disease-associated variations comprise GWAS variants. In some embodiments, the disease-associated variations comprise GWAS variants in a super-enhancer associated with the core regulatory circuitry in the cell or tissue of interested selected from the group consisting of i) at least one gene encoding a master transcription factor, (ii) the master transcription factor encoded by the at least one gene, or (iii) at least one target of the master transcription factor. In some embodiments, the GWAS variant is selected from the group consisting of (i) a GWAS variant from Alzheimer disease present in the cell identity program of brain hippocampus; (ii) a GWAS variant from systemic lupus erythematosus present in the cell identity program of CD20 cells; (iii) a GWAS variant from fasting insulin trait present in the cell identity program of adipose nuclei; (iv) a GWAS variant from ulcerative colitis present in the cell identity program of sigmoid colon; and (vi) a GWAS variant from electrocardiographic traits present in the cell identity program of left ventricle.

In some aspects, the disclosure provides a method of treating a cell identity program-related disorder in a subject in need thereof, comprising modulating at least one abnormal component of a cell identity program in a diseased cell or tissue of the subject.

In some embodiments, modulating at least one abnormal component of the cell identity program in the diseased cell or tissue of the subject comprises administering to the subject an effective amount of an agent that modulates the at least one abnormal component of the cell identity program. In some embodiments, the agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof. In some embodiments, the diseased cell or tissue comprises a tumor cell or tissue. In some embodiments, the diseased cell or tissue comprises a cell or tissue listed in Table 2, and the abnormal component comprises at least one component of the cell identity program of the cell listed in Table 2 selected from the group consisting of (i) a gene encoding a master transcription factor, (ii) the master transcription factor encoded by the gene, (iii) a target of the master transcription factor, and (iv) a super-enhancer associated with any of (i)-(iii), or a component of the super-enhancer.

In some embodiments, the method further includes diagnosing the subject as having the cell identity program-related disorder.

In some aspects, the disclosure provides a method of reprogramming a cell of a first cell type to a cell of a second cell type, the method comprising modulating at least one component of the core regulatory circuitry of the second cell type in the cell of the first cell type.

In some embodiments, the (i) the at least one component comprises a transcriptional repressor or transcriptional co-repressor and modulating comprises repressing the at least one component; and/or (ii) the at least one component comprises a transcriptional activator or transcriptional co-activator and modulating comprises activating the at least one component. In some embodiments, activating the at least one component comprises (i) expressing the at least one component of the core regulatory circuitry of the second cell type in the cell of the first type; (ii) introducing the at least one component of the core regulatory circuitry of the second cell type into the cell of the second type; (iii) contacting the cell with an agent that activates expression of the at least one component of the core regulatory circuitry of the second cell type in the cell of the first type; and (iv) any combination of (i)-(iii). In some embodiments, modulating (e.g., activating) the at least one component of the core regulatory circuitry of the second cell type in the cell of the first type occurs ex vivo. In some embodiments, modulating (e.g., repressing) the at least one component of the core regulatory circuitry of the second cell type in the cell of the first type occurs ex vivo.

In some embodiments, modulating (e.g., activating) the at least one component of the core regulatory circuitry of the second cell type in the cell of the first type occurs in vivo. In some embodiments, modulating (e.g., repressing) the at least one component of the core regulatory circuitry of the second cell type in the cell of the first type occurs in vivo.

In some embodiments, the method includes inhibiting at least one component of the core regulatory circuitry of the first cell type. In some embodiments, the (i) cell of the first cell type comprises the core regulatory circuitry of a diseased cell, and the cell of the second cell type comprises the core regulatory circuitry of a normal cell; (ii) cell of the first cell type comprises the core regulatory circuitry of a terminally differentiated cell, and the cell of the second cell type comprises the core regulatory circuitry of a less differentiated cell; (iii) cell of the first cell type comprises the core regulatory circuitry of a first somatic cell type, and the cell of the second cell type comprises the core regulatory circuitry of a second somatic cell type; (iv) cell of the first cell type comprises the core regulatory circuitry of a somatic cell, and the cell of the second cell type comprises the core regulatory circuitry of an embryonic cell; (v) cell of the first cell type comprises the core regulatory circuitry of a first tissue type, and the cell of the second type comprises the core regulatory circuitry of a second tissue type; (vi) cell of the first cell type comprises the core regulatory circuitry of a skin or fat cell, and the cell of the second cell type comprises the core regulatory circuitry of a tissue; and (vii) cell of the first cell type comprises the core regulatory circuitry of a tumor cell or tissue, and the cell of the second cell type comprises the core regulatory circuitry of a healthy cell or tissue.

In some aspects, the disclosure provides a method of identifying a candidate modulator of at least one component of the core regulatory circuitry of a cell or tissue, comprising: a) contacting a cell or tissue with a test agent; and b) assessing the ability of the test agent to modulate at least one component of the core regulatory circuitry of the cell or tissue, wherein the test agent is identified as a candidate modulator of the at least one component of the core regulatory circuitry of the cell or tissue if the at least one component of the core regulatory circuitry is activated or inhibited in the presence of the test agent.

In some embodiments, the at least one component of the core regulatory circuitry of the cell or tissue comprises a reprogramming factor or a cell identity gene. In some embodiments, the at least one component of the core regulatory circuitry of the cell or tissue comprises a disease-associated variant.

In some aspects, the disclosure provides a method of reprogramming a cell comprising contacting the cell with the candidate modulator identified according to a method described herein. In some embodiments, at least one component of the core regulatory circuitry of the cell comprises a disease-associated variant. In some embodiments, contacting occurs in vivo or ex vivo.

In some aspects, the disclosure provides a method of identifying a candidate modulator of at least one component of the cell identity program of a cell or tissue, comprising: a) contacting a cell or tissue with a test agent; and b) assessing the ability of the test agent to modulate at least one component of the cell identity program of the cell or tissue, wherein the test agent is identified as a candidate modulator of the at least one component of the cell identity program of the cell or tissue if the at least one component of the cell identity program of the cell or tissue is activated or inhibited in the presence of the test agent.

In some embodiments, the at least one component of the cell identity program of the cell or tissue comprises a reprogramming factor or a cell identity gene. In some embodiments, the at least one component of the cell identity program of the cell or tissue comprises a disease-associated variant.

In some aspects, the disclosure provides a method of reprogramming a cell comprising contacting the cell with the candidate modulator identified according to a method described herein. In some embodiments, at least one component of the core regulatory circuitry of the cell comprises a disease-associated variant. In some embodiments, contacting occurs in vivo or ex vivo.

In some aspects, the disclosure provides a method of identifying a target for drug discovery comprising identifying a variation in at least one component of the core regulatory circuitry of a cell or tissue that is more prevalent in subjects suffering from a disease than in healthy subjects, wherein the at least one component of the core regulatory circuitry of the cell or tissue that is more prevalent in subjects suffering from a disease than in healthy subjects comprises a disease-associated variant, and wherein the disease-associated variant is a target for drug discovery.

In some aspects, the disclosure provides a method of identifying a target for drug discovery comprising identifying a variation in at least one component of the cell identity program of a cell or tissue that is more prevalent in subjects suffering from a disease than in healthy subjects, wherein the at least one component of the cell identity program of the cell or tissue that is more prevalent in subjects suffering from a disease than in healthy subjects comprises a disease-associated variant, and wherein the disease-associated variant is a target for drug discovery.

In some embodiments, the target for drug discovery comprises a target for diagnostic purposes.

In some aspects, the disclosure provides a method of identifying a target for anti-cancer drug discovery comprising: a) comparing the core regulatory circuitry of a tumor cell or tissue with the core regulatory circuitry of a corresponding non-tumor cell or tissue; and b) identifying at least one component that differs between the core regulatory circuitry of the tumor cell or tissue and the corresponding non-tumor cell or tissue, wherein the at least one component that differs between the core regulatory circuitry of the tumor cell or tissue and the corresponding non-tumor cell or tissue is identified as a target for anti-cancer drug discovery.

In some embodiments, a gene regulated by the at least one component is identified as a target for anti-cancer drug discovery. In some embodiments, the at least one component differs in sequence, expression, and/or activity.

In some aspects, the disclosure provides a method of identifying an anti-cancer agent comprising identifying a modulator of the target for anti-cancer drug discovery identified according to a method described herein.

In some aspects, the disclosure provides a method treating a cancer characterized by tumor cell or tissue comprising the target for anti-cancer drug discovery, comprising administering to a subject suffering from the cancer an effective amount of the anti-cancer agent identified according to a method described herein.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-ID depict schematics of the inventive method. FIG. 1A is a schematic depicting the identification of master transcription factor candidates. FIG. 1B is a schematic depicting the identification of predicted auto-regulated transcription factors. FIG. 1C is a schematic depicting the assembly of core regulatory circuits. FIG. 1D is a schematic depicting a model of the core regulatory circuitry in human embryonic stem cells (ESCs).

FIGS. 2A-2C depict schematics of the inventive method. FIG. 2A is a schematic demonstrating that master transcription factors form autoregulatory loops. FIG. 2B is a schematic depicting the identification of predicted master transcription factor target genes. FIG. 2C is a schematic illustrating a cell identity program map of human embryonic stem cells.

FIG. 3 shows clustering of the predicted master transcription factors in 43 human cell types.

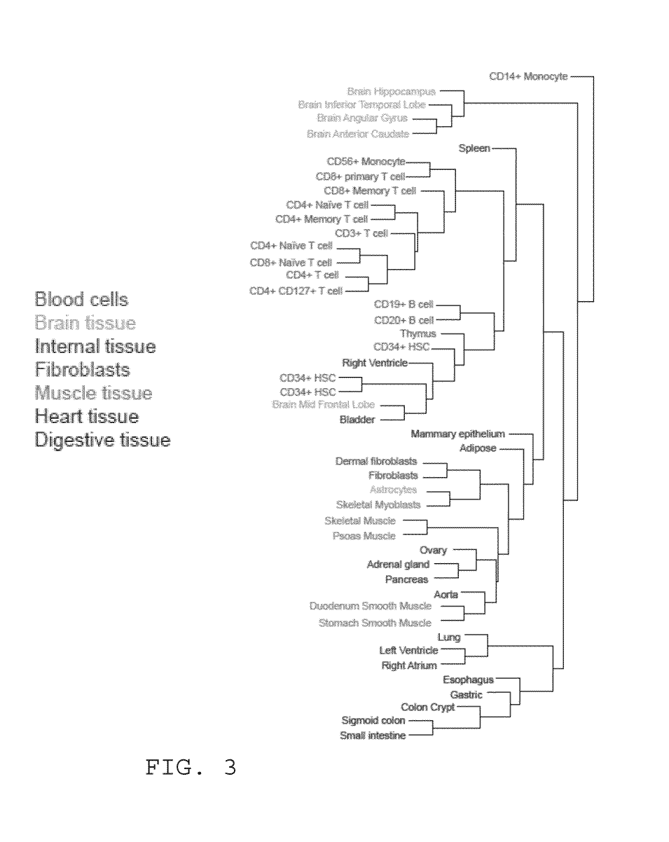

FIG. 4 is a schematic demonstrating that GWAS variants are enriched in regulatory regions of the cell identity programs of multiple disease relevant cell types. Super-enhancers containing GWAS variants are depicted. Brain: GWAS variants from Alzheimer disease have been mapped on Brain Hippocampus middle circuitry; Blood: GWAS variants from Systemic Lupus Erythematosus have been mapped on CD20 circuitry; Fat: GWAS variants from fasting insulin trait have been mapped on Adipose nuclei circuitry; Colon: GWAS variants from ulcerative colitis have been mapped on sigmoid colon circuitry; Heart: GWAS variants from Electrocardiographic traits have been mapped to left ventricle circuitry.

FIG. 5 demonstrates systemic lupus erythematosus-associated variation in the B cell CRC identity program.

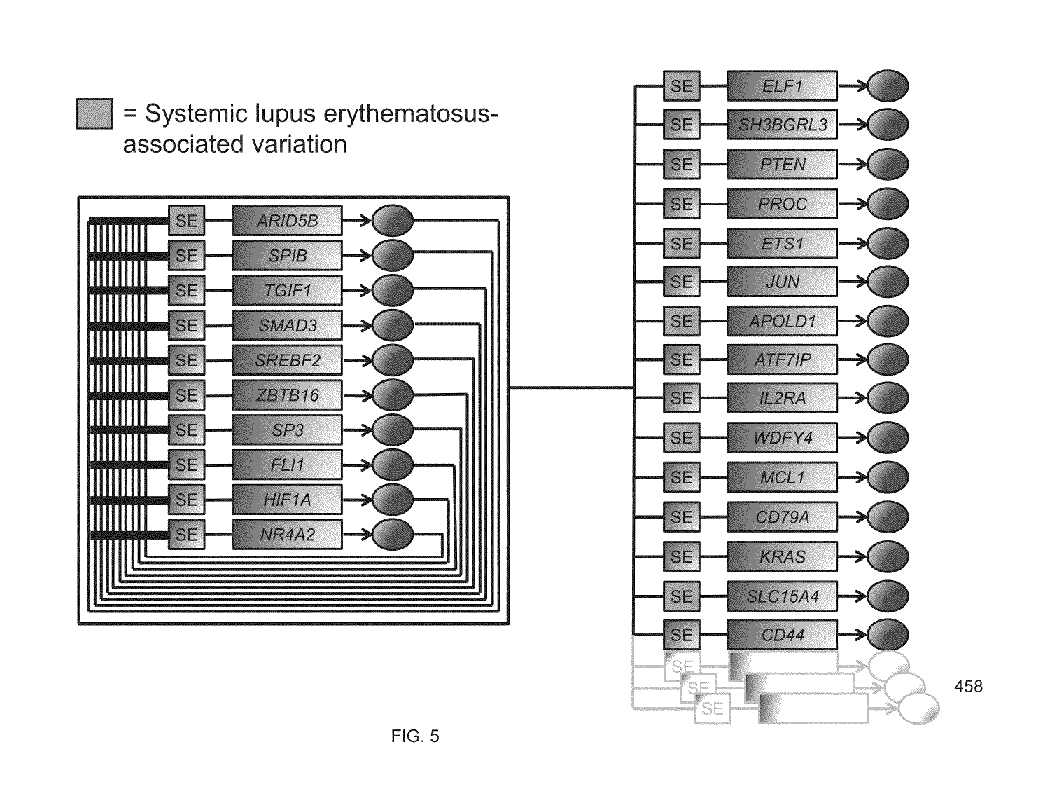

### DETAILED DESCRIPTION OF THE INVENTION

Aspects of the disclosure relate to methods of identifying the core regulatory circuitry and/or cell identity programs of cells or tissues, and related diagnostic, treatment, and screening methods involving the core regulatory circuitry and/or cell identity programs identified.

In embryonic stem cells and a few other cell types, master transcription factors (TFs) have been shown to function together in a core regulatory circuit (CRC) that controls the gene expression programs that define cell identity (Boyer et al., 2005; Lee and Young, 2011; Odom et al., 2006; Lien et al., 2002; Novershtern et al., 2011). In these CRCs, the master TFs regulate their own genes and other genes key to cell identity though their binding of the super-enhancers associated with those genes (Whyte et al., 2013; Hnisz et al., 2013). Work described herein exploits novel features of super-enhancers and TF binding site sequences for 43 cell types and tissues to construct models of CRCs for a broad spectrum of cell types throughout the human body. Cell Identity Program models for these cells, which consist of the master TFs forming the CRCs and their target genes, contain the vast majority of master TFs and reprogramming factors described for specific cell types in the literature and cluster according to known cell lineages. The work described herein also demonstrates that the master TFs in the CRCs have binding site sequences in the enhancers of the majority of cell identity genes that are expressed in each cell/tissue type. Surprisingly, the work described herein also demonstrates that the regulatory elements within the Cell Identity Program models are highly enriched in disease-associated sequence variation, and shows how tumor cells can modify the CRC to create gene expression programs associated with tumor pathology. These maps of core regulatory circuitry provide founding models to test and expand knowledge of regulatory circuitry, provide guidance for reprogramming studies, and should facilitate understanding of disease causality.

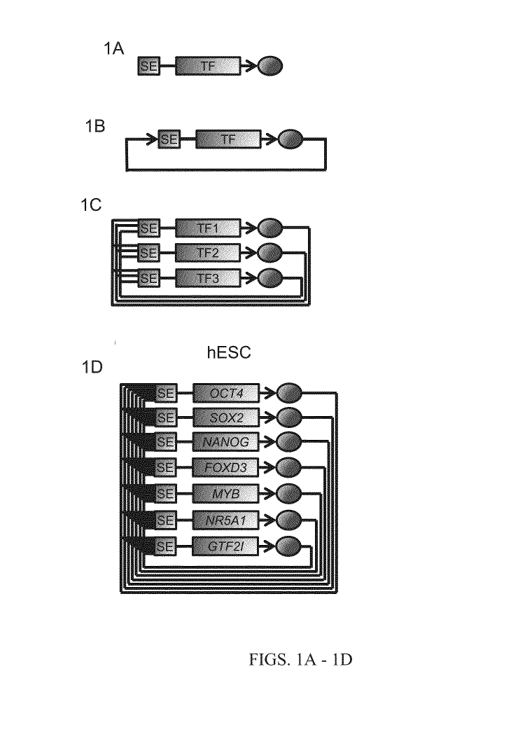

Accordingly, aspects of the disclosure relate to methods for identifying the core regulatory circuitry of a cell or tissue. In some aspects, a method of identifying the core regulatory circuitry of a cell or tissue comprises: a) identifying a group of transcription factor encoding genes in a cell or tissue which are associated with a super-enhancer; b) determining which transcription factor encoding genes identified in a) comprise autoregulated transcription factor encoding genes, wherein a transcription factor encoding gene identified in a) comprises an autoregulated transcription factor encoding gene if a transcription factor encoded by the transcription factor encoding gene is predicted to bind to a super-enhancer associated with the transcription factor encoding gene; and c) identifying the core regulatory circuitry of the cell or tissue, wherein the core regulatory circuitry of the cell or tissue comprises autoregulated transcription factor encoding genes identified in b) which form an interconnected autoregulatory loop, wherein the autoregulated transcription factor encoding genes identified in b) form an interconnected autoregulatory loop if each transcription factor encoded by an autoregulated transcription factor encoding gene identified in b) is predicted to bind to a super-enhancer associated with each of the other autoregulated transcription factor encoding genes identified in b). An exemplary embodiment of a method for identifying the core regulatory circuitry of a cell or tissue is depicted in FIGS. 1A, 1B, 1C, and ID.

As is shown in the example embodiment depicted in FIG. 1A, master transcription factor candidates are identified in a cell or tissue by determining all of the transcription factors in the cell or tissue which are encoded by genes associated with a super-enhancer in the cell or tissue, e.g., the group of transcription factor encoding genes associated with a super-enhancer. As used herein, a “transcription factor encoding gene” refers to any gene which encodes a transcription factor. The transcription factor can be a known transcription factor, a putative transcription factor, etc. . . . . It should be appreciated that the group of transcription factor encoding genes is intended to encompass all genes in a particular cell or tissue which encode master transcription factors. The number of such transcription factor encoding genes may vary depending on the particular cell or tissue type. In some embodiments, the group of transcription factor encoding genes (e.g., genes encoding master transcription factors) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 transcription factor encoding genes. In some embodiments, the group of transcription factor encoding genes comprises at least 5, at least 10, at least 15, at least 20, at least 25, at least 30, at least 35, at least 40, at least 45, or at least 50 transcription factor encoding genes. In some embodiments, the group of transcription factor encoding genes comprise at least 50, at least 60, at least 70, at least 80, at least 90, or at least 100 transcription factor encoding genes.

As is illustrated in FIG. 1B, the master transcription factor candidates identified in step a) (e.g., as exemplified in FIG. 1A) can then be assessed in step b) to determine whether the master transcription factor candidates are autoregulated transcription factors. As used herein, the phrase “autoregulated transcription factor” refers to a transcription factor encoded by an autoregulated transcription factor encoding gene, i.e., a super-enhancer associated with the transcription factor encoding gene is predicted to be bound by the transcription factor encoded by the transcription factor encoding gene. Put differently, as is shown in FIG. 1B, the transcription factor encoding gene (boxed TF) encodes a transcription factor (oval) that binds to the super-enhancer (boxed SE) associated with the transcription actor encoding gene. It is expected that only a fraction of the candidate master transcription factors in any particular cell or tissue will comprise autoregulated transcription factors. In some embodiments, at least 1%, at least 2%, at least 3%, at least 4%, at least 5%, at least 6%, at least 7%, at least 8%, at least 9%, or at least 10% of the candidate master transcription factors in a cell or tissue comprise autoregulated transcription factors. In some embodiments, at least 1%, at least 2%, at least 3%, at least 4%, at least 5%, at least 6%, at least 7%, at least 8%, at least 9%, or at least 10% of the super-enhancer associated transcription factor encoding genes in a cell or tissue comprise autoregulated transcription factor encoding genes.

As exemplified in the embodiment shown in FIG. 1C, step c) of the method involves identifying a core regulatory circuitry of the cell or tissue by determining the largest set of fully interconnected autoregulated transcription factors or autoregulated transcription factor encoding genes identified in step b) which forms an interconnected autoregulatory loop. As used herein, the phrases “autoregulated transcription factors forming an interconnected autoregulatory loop” and “master transcription factors” are used interchangeably herein to refer to transcription factors encoded by genes whose expression is driven by super-enhancers, and which bind their own super-enhancers (e.g., a super-enhancer or super-enhancer component associated with the gene encoding the transcription factor) as well as super-enhancers associated with other autoregulated transcription factor encoding genes and/or the transcription factors encoded by those genes in the interconnected autoregulatory loop.

As used herein, the phrase “interconnected autoregulatory loop” refers to a network of autoregulated transcription factor encoding genes predicted to bind each of the super-enhancers associated with other autoregulated transcription factors in the network. The concept of an autoregulatory loop is depicted in FIG. 1C for three hypothetical transcription factors TF1, TF2, TF3. As shown in FIG. 1C, the interconnected autoregulatory loop forms a core regulatory circuitry that includes each autoregulated transcription factor encoding gene (e.g., TF1, TF2, and TF3), the autoregulated transcription factor encoded by each autoregulated transcription factor encoding gene (e.g., oval 1, oval 2, and oval 3), the super-enhancers or a component of a super-enhancer associated with each autoregulated transcription factor encoding gene, wherein each autoregulated transcription factor in the network is predicted to bind to or binds to each super-enhancer in the network. To further illustrate the core regulatory circuitry concept, FIG. 1D depicts a model of the core regulatory circuitry in human embryonic stem cells (ESCs). In some embodiments, the core regulatory circuitry comprises the autoregulated transcription factors forming the interconnected autoregulatory loop, the transcription factors encoded by the autoregulated transcription factor encoding genes, a super-enhancers associated with the autoregulated transcription factor encoding genes, or a component of the super-enhancer. In some embodiments, a component of the core regulatory circuitry comprises a transcriptional activator, i.e., a component whose activation favors activation of the overall core regulatory circuitry of a cell or tissue. In some embodiments, a component of the core regulatory circuitry comprises a transcriptional repressor, i.e., a component whose repression favors activation of the overall core regulatory circuitry of a cell or tissue.

As used herein, the phrase “super-enhancer” refers to clusters of enhancers which drive the expression of genes encoding the master transcription factors and other genes key to cell identity. The disclosure contemplates the use of any super-enhancer. Exemplary super-enhancers are disclosed in PCT International Application No. PCT/US2013/066957 (attorney docket no. WIBR-137-WO1), filed Oct. 25, 2013, the entirety of which is incorporated by reference herein.

As used herein, the phrase “super-enhancer component” refers to a component, such as a protein, that has a higher local concentration, or exhibits a higher occupancy, at a super-enhancer, as opposed to a normal enhancer or an enhancer outside a super-enhancer, and in embodiments, contributes to increased expression of the associated gene. In an embodiment, the super-enhancer component is a nucleic acid (e.g., RNA, e.g., eRNA transcribed from the super-enhancer, i.e., an eRNA). In an embodiment, the nucleic acid is not chromosomal nucleic acid. In an embodiment, the component is involved in the activation or regulation of transcription. In some embodiments, the super-enhancer component comprises RNA polymerase II, Mediator, cohesin, Nipbl, p300, CBP, Chd7, Brd4, and components of the esBAF (Brg1) or a Lsd1-Nurd complex (e.g., RNA polymerase II).

As used herein, “enhancer” refers to a short region of DNA to which proteins (e.g., transcription factors) bind to enhance transcription of a gene. As used herein, “transcriptional coactivator” refers to a protein or complex of proteins that interacts with transcription factors to stimulate transcription of a gene. In some embodiments, the transcriptional coactivator is Mediator. In some embodiments, the transcriptional coactivator is Med1 (Gene ID: 5469). In some embodiments, the transcriptional coactivator is a Mediator component. As used herein, “Mediator component” comprises or consists of a polypeptide whose amino acid sequence is identical to the amino acid sequence of a naturally occurring Mediator complex polypeptide. The naturally occurring Mediator complex polypeptide can be, e.g., any of the approximately 30 polypeptides found in a Mediator complex that occurs in a cell or is purified from a cell (see, e.g., Conaway et al., 2005; Kornberg, 2005; Malik and Roeder, 2005). In some embodiments a naturally occurring Mediator component is any of Med1-Med 31 or any naturally occurring Mediator polypeptide known in the art. For example, a naturally occurring Mediator complex polypeptide can be Med6, Med7, Med10, Med12, Med14, Med15, Med17, Med21, Med24, Med27, Med28 or Med30. In some embodiments a Mediator polypeptide is a subunit found in a Med11, Med17, Med20, Med22, Med 8, Med 18, Med 19, Med 6, Med 30, Med 21, Med 4, Med 7, Med 31, Med 10, Med 1, Med 27, Med 26, Med14, Med15 complex. In some embodiments a Mediator polypeptide is a subunit found in a Med12/Med13/CDK8/cyclin complex. Mediator is described in further detail in PCT International Application No. WO 2011/100374, the teachings of which are incorporated herein by reference in their entirety.

In some embodiments, the method of identifying the core regulatory circuitry comprises d) determining at least one target of at least one transcription factor encoded by at least one autoregulated transcription factor encoding gene. In some embodiments, the at least one target of the at least one transcription factor encoded by the at least one autoregulated transcription factor encoding gene comprises a gene which encodes a reprogramming factor or a cell identity gene.

Any suitable method can be used to determine whether the transcription factor encoded by the transcription factor encoding gene is predicted to bind to the super-enhancer associated with the transcription factor encoding gene, e.g., motif analysis or searching. In some embodiments, the transcription factor encoded by the transcription factor encoding gene is predicted to bind to the super-enhancer associated with transcription factor encoding gene if the super-enhancer associated with the transcription factor encoding gene comprises at least one DNA sequence motif predicted for the transcription factor encoded by the transcription factor encoding gene. In some embodiments, each transcription factor encoded by the autoregulated transcription factor encoding gene is predicted to bind to the super-enhancer associated with each of the other autoregulated transcription factor encoding genes if the super-enhancers associated with each of the other autoregulated transcription factor encoding genes comprise at least one DNA sequence motif predicted for each of the transcription factors encoded by each of the other autoregulated transcription factor encoding genes.

The at least one DNA sequence motif can be located within any range upstream or downstream of the super-enhancer associated with the transcription factor encoding gene (e.g., autoregulated transcription factor encoding gene). In some embodiments, the at least one DNA sequence motif is located between 10,000 bp upstream and 10,000 bp downstream of the super-enhancer associated with the transcription factor encoding gene. In some embodiments, the at least one DNA sequence motif is located between 5,000 bp upstream and 5,000 bp downstream of the super-enhancer associated with the transcription factor encoding gene. In some embodiments, the at least one DNA sequence motif is located between 500 bp upstream and 500 bp downstream of the super-enhancer associated with the transcription factor encoding gene. In some embodiments, the at least one DNA sequence motif is located between 50 bp upstream and 50 bp downstream of the super-enhancer associated with the transcription factor encoding gene.

In some embodiments, the methods described herein comprise obtaining ChIP-seq data for histone H3K27Ac, e.g., as a marker of an enhancer, e.g., a super-enhancer associated with a transcription factor encoding gene. In some embodiments, the H3K27Ac ChIP-seq data can be used to create a catalogue of super-enhancers for a cell or tissue of interest described herein.

Aspects of the disclosure involve cells of interest. The disclosure contemplates any cell of interest. In some embodiments, the cell comprises a cell of ectoderm lineage. In some embodiments, the cell comprises a cell of endoderm lineage. In some embodiments, the cell comprises a cell of mesoderm lineage. In some embodiments, the cell comprises an embryonic cell (e.g., embryonic stem cell). In some embodiments, the cell comprises a pluripotent cell (e.g., an induced pluripotent stem cell). In some embodiments, the cell comprises a somatic cell. In some embodiments, the cell comprises a multipotent cell. In some embodiments, the cell comprises a progenitor cell. In some embodiments, the cell comprises a cell listed in Table 1. In some embodiments, the cell comprises a cell listed in Table 2. In some embodiments, the cell comprises a) a blood cell selected from the group consisting of a CD14+ monocyte, a CD56+ monocyte, a CD4+ T cell, a CD3+ T cell, a CD4+ primary T cell, a CD4+ memory T cell, a CD4+ naïve T cell, a CD4+CD127+ T cell, a CD8+ primary T cell, a CD8+ memory T cell, a CD8+ naïve T cell, a CD19+ B cell, a CD20+ B cell, a CD34+ HSC cell; b) a brain cell selected from the group consisting of astrocytes, glial cells, an neurons; c) a fibroblast selected from the group consisting of dermal fibroblast and fibroblast; d) skeletal myoblasts; e) a colon crypt, f) an embryonic stem cell; g) a hepatocyte; h) a tumor cell; i) a keratinocyte; j) a macrophage; k) lymphocytes; I) regulatory T (Tregs); m) NK cells; n) pancreatic beta cells; o) cardiac muscle cells; p) nerve cells; and q) chondrocytes (e.g., for cartilage repair).

In some embodiments, the cell comprises a diseased cell. In some embodiments, the cell comprises a cell that harbors a disease-associated variant (e.g., a GWAS variant). In some embodiments, the tumor cell is a cell from a cancer selected from the group consisting of ovarian cancer, bladder cancer, lung cancer, cervical cancer, breast cancer, prostate cancer, gliomas, fibrosarcomas, retinoblastomas, melanomas, soft tissue sarcomas, osteosarcomas, leukemias, stomach cancer, colon cancer, carcinoma of the kidney, gastrointestinal cancer, salivary gland cancer, pancreatic cancer, Hodgkin's disease, non-Hodgkin's lymphomas, acute and chronic lymphocytic leukemias, multiple myeloma, neuroblastoma, Wilms' tumor, testicular cancer, soft-tissue sarcomas, chronic lymphocytic leukemia, primary macroglobulinemia, chronic granulocytic leukemia, primary brain carcinoma, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

Aspects of the disclosure involve tissues of interest. The disclosure contemplates any tissue of interest. In some embodiments, the tissue comprises tissue of mesoderm lineage. In some embodiments, the tissue comprises tissue of endoderm lineage. In some embodiments, the tissue comprises tissue of ectoderm lineage. In some embodiments, the tissue comprises germ tissue. In some embodiments, the tissue comprises a) brain tissue selected from the group consisting of brain hippocampus, brain inferior temporal lobe, brain angular gyrus, and brain mid frontal lobe; b) internal tissue selected from the group consisting of spleen, bladder, mammary epithelium, adipose, ovarian, adrenal gland, pancreatic, and lung; d) thymus; e) muscle tissue selected from the group consisting of skeletal muscle, psoas muscle, duodenum smooth muscle, and stomach smooth muscle; f) heart tissue selected from the group consisting of right ventricle, aorta, left ventricle, and right atrium; g) digestive tissue selected from the group consisting of esophagus, gastric, sigmoid colon, and small intestine; and h) tumor tissue.

In an embodiment the sample includes a cell or tissue, e.g., a cell or tissue from any of human cells; fetal cells; embryonic stem cells or embryonic stem cell-like cells, e.g., cells from the umbilical vein, e.g., endothelial cells from the umbilical vein; muscle, e.g., myotube, fetal muscle; blood cells, e.g., cancerous blood cells, fetal blood cells, monocytes; B cells, e.g., Pro-B cells; brain, e.g., astrocyte cells, angular gyrus of the brain, anterior caudate of the brain, cingulate gyrus of the brain, hippocampus of the brain, inferior temporal lobe of the brain, middle frontal lobe of the brain, brain cancer cells; T cells, e.g., naïve T cells, memory T cells; CD4 positive cells; CD25 positive cells; CD45RA positive cells; CD45RO positive cells; IL-17 positive cells; cells stimulated with PMA; Th cells; Th17 cells; CD255 positive cells; CD127 positive cells; CD8 positive cells; CD34 positive cells; duodenum, e.g., smooth muscle tissue of the duodenum; skeletal muscle tissue; myoblast; stomach, e.g., smooth muscle tissue of the stomach, e.g., gastric cells; CD3 positive cells; CD14 positive cells; CD19 positive cells; CD20 positive cells; CD34 positive cells; CD56 positive cells; prostate, e.g., prostate cancer; colon, e.g., colorectal cancer cells; crypt cells, e.g., colon crypt cells; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cells; skin, e.g., fibroblast cells; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer.

In some embodiments, the tumor tissue is tumor tissue from a cancer selected from the group consisting of ovarian cancer, bladder cancer, lung cancer, cervical cancer, breast cancer, prostate cancer, gliomas, fibrosarcomas, retinoblastomas, melanomas, soft tissue sarcomas, osteosarcomas, leukemias, stomach cancer, colon cancer, carcinoma of the kidney, gastrointestinal cancer, salivary gland cancer, pancreatic cancer, Hodgkin's disease, non-Hodgkin's lymphomas, acute and chronic lymphocytic leukemias, multiple myeloma, neuroblastoma, Wilms' tumor, testicular cancer, soft-tissue sarcomas, chronic lymphocytic leukemia, primary macroglobulinemia, chronic granulocytic leukemia, primary brain carcinoma, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

In some embodiments, the cell or tissue of interest comprises a cell or tissue that is affected by a disease. Exemplary diseases include, without limitation, an autoimmune disease, a metabolic disease, a cardiovascular disease, a neurological disease, a psychiatric disease, a renal disease, a liver disease, a dermatological disease, a pancreatic disease, a glandular disease, a lymph disease, an ophthalmological disease, an orthopedic disease, an inflammatory disease, a hematological disease, an infectious disease, a cell-type specific disease, an olfactory disease, etc. In some embodiments, the cell or tissue affected by a disease is obtained from a subject suffering from the disease.

Aspects of the disclosed methods include obtaining a biological sample from a subject comprising a cell or tissue of interest. A biological sample used in the methods described herein will typically comprise or be derived from cells or tissues isolated from a subject. The cells or tissues may comprise cells or tissues affected by a disease described herein. In some embodiments, the cells or tissues are isolated from a tumor cell or tissue described herein.

Samples can be, e.g., surgical samples, tissue biopsy samples, fine needle aspiration biopsy samples, core needle samples. The sample may be obtained using methods known in the art. A sample can be subjected to one or more processing steps. In some embodiments the sample is frozen and/or fixed. In some embodiments the sample is sectioned and/or embedded, e.g., in paraffin. In some embodiments, tumor cells, e.g., epithelial tumor cells, are separated from at least some surrounding stromal tissue (e.g., stromal cells and/or extracellular matrix). Cells or tissue of interest can be isolated using, e.g., tissue microdissection, e.g., laser capture microdissection. It should be appreciated that a sample can be a sample isolated from any of the subjects described herein.

In some embodiments, cells of the sample are lysed. Nucleic acids or polypeptides may be isolated from the samples (e.g., cells or tissues of interest). In some embodiments DNA, optionally isolated from a sample, is amplified. A wide variety of methods are available for detection of DNA, e.g., DNA of super-enhancers associated with autoregulated transcription factor encoding genes, DNA of an autoregulated transcription factor encoding gene, a DNA sequence motif, etc. In some embodiments RNA, optionally isolated from a sample, is reverse transcribed and/or amplified. A wide variety of solution phase or solid phase methods are available for detection of RNA, e.g., mRNA encoding a master transcription factor or autoregulated transcription factor, mRNA encoding a target of a master transcription factor. Suitable methods include e.g., hybridization-based approaches (e.g., nuclease protection assays, Northern blots, microarrays, in situ hybridization), amplification-based approaches (e.g., reverse transcription polymerase chain reaction (which can be a real-time PCR reaction), or sequencing (e.g., RNA-Seq, which uses high throughput sequencing techniques to quantify RNA transcripts (see, e.g., Wang, Z., et al. Nature Reviews Genetics 10, 57-63, 2009)). In some embodiments of interest a quantitative PCR (qPCR) assay is used. Other methods include electrochemical detection, bioluminescence-based methods, fluorescence-correlation spectroscopy, etc.

Aspects of the methods described herein involve detecting the levels or presence of expression products, e.g., an expression product of a component the core regulatory circuitry comprising a disease associated variation (e.g., such as a single nucleotide polymorphism), an autoregulated transcription factor, an expression product of a target gene of a master transcription factor, etc.). Levels of expression products, e.g., of master transcription factor target genes, may be assessed using any suitable method. Either mRNA or protein level may be measured. A “polypeptide”, “peptide” or “protein” refers to a molecule comprising at least two covalently attached amino acids. A polypeptide can be made up of naturally occurring amino acids and peptide bonds and/or synthetic peptidomimetic residues and/or bonds. Polypeptides described herein include naturally purified products, products of chemical synthetic procedures, and products produced by recombinant techniques from a prokaryotic or eukaryotic host, including, for example, bacterial, yeast, higher plant, insect and mammalian cells.

Exemplary methods for measuring mRNA include hybridization based assays, polymerase chain reaction assay, sequencing, in situ hybridization, etc. Exemplary methods for measuring protein levels include ELISA assays, Western blot, mass spectrometry, or immunohistochemistry. It will be understood that suitable controls and normalization procedures can be used to accurately quantify expression. Values can also be normalized to account for the fact that different samples may contain different proportions of a cell type of interest, e.g., tumor cells or tissues compared to corresponding non-tumor cells or tissues (e.g., health cells or tissues).

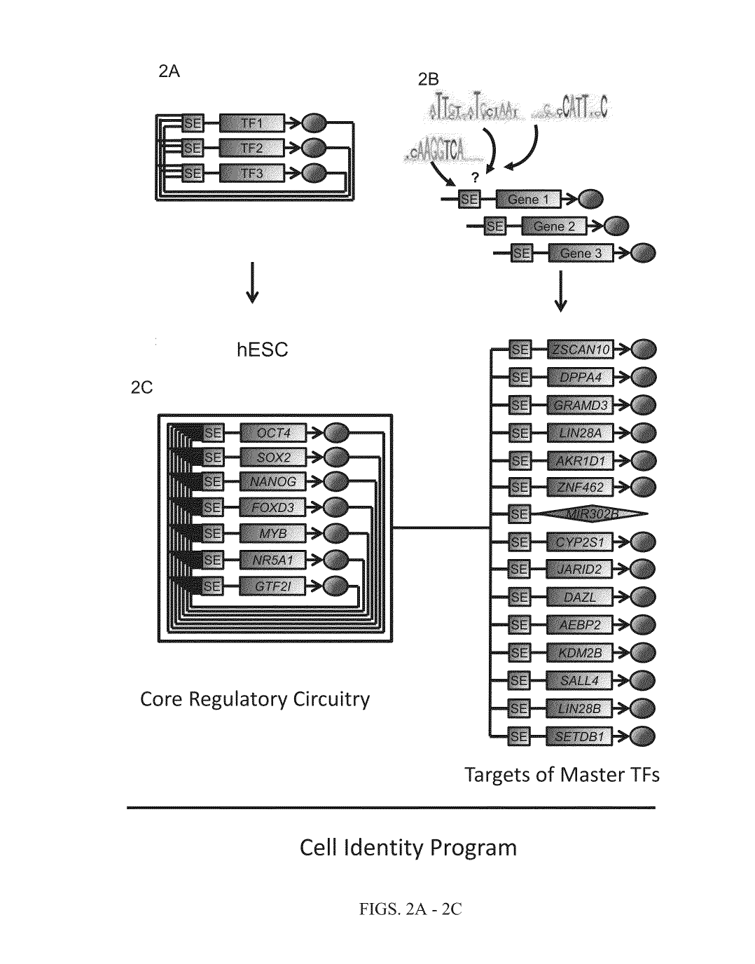

Aspects of the disclosure relate to methods of identifying the cell identity program of a cell or tissue. Generally, the methods of identifying the cell identity program of a cell or tissue incorporate the methods of identifying the core regulatory circuitry and extend those methods according to exemplary embodiments depicted in FIGS. 2A, 2B, and 2C. FIG. 2A is a schematic demonstrating that master transcription factors form autoregulatory loops. FIG. 2B is a schematic depicting the identification of predicted master transcription factor target genes. FIG. 2C is a schematic illustrating a cell identity program map of human embryonic stem cells.

In some aspects, a method of identifying the cell identity program of a cell or tissue, comprising a) identifying the core regulatory circuitry of a cell or tissue of interest, wherein the core regulatory circuitry of the cell or tissue of interest comprises at least one autoregulated transcription factor encoding gene associated with a super-enhancer in the cell or tissue of interest, at least one transcription factor encoded by the at least one autoregulated transcription factor encoding gene, at least one super-enhancer associated with the at least one autoregulated transcription factor encoding gene, and optionally at least one component of the super-enhancer; and b) identifying the cell identity program of the cell or tissue, wherein the cell identity program of the cell or tissue comprises the core regulatory circuitry identified in a) and at least one target of the at least one transcription factor encoded by the at least one autoregulated transcription factor encoding gene in the core regulatory circuitry.

As used herein, the phrase “cell identity program” refers to the core regulatory circuitry of a cell or tissue and targets of master transcription factors that are part of the core regulatory circuitry of the cell or tissue, as is depicted in FIG. 2C, which shows an exemplary a cell identity program of human embryonic stem cells.

The disclosure contemplates the use of any target of a master transcription factor that is part of the core regulatory circuitry of a cell or tissue, e.g., at least one target which comprises a gene comprising at least one enhancer element predicted to be bound by the at least one transcription factor. In some embodiments, the at least one enhancer element predicted to be bound by the at least one transcription factor comprises a DNA sequence motif associated with a super-enhancer.

Surprisingly, and unexpectedly, the work described herein demonstrates the cell identity programs constructed for 43 different human cell and tissue types. Exemplary cell identity programs for 43 different human cell and tissue types are shown in Table 2.

Aspects of the disclosure relate to methods for modulating cell identity. Generally, the methods of modulating cell identity disclosed herein involve modulating at least one component of a cell identity program of a cell. The at least one component of the cell identity program in the cell comprises the core regulatory circuitry of the cell or at least one target modulated by the at least one component of the core regulatory circuitry of the cell. The disclosure contemplates the use of any suitable method for modulating the at least one component of a cell identity program of a cell. In some embodiments, modulating the at least one component of the cell identity program in the cell comprises contacting the cell with an agent that modulates at least one component of the cell identity program of the cell. The expressions “activate”, “inhibit”, “modulate”, “increase”, “decrease” or the like, e.g., which denote quantitative differences between two states, refer to at least statistically significant differences between the two states. For example, “modulating at least one component of the cell identity program” means that the sequence, expression, or activity of the at least one component of the cell identity program is modified, activated, increased, inhibited, or decreased in the presence of the agent by at least statistically significantly amount compared to the sequence, expression, or activity of the at least one component of the cell identity program in the absence of the agent. Such terms are applied herein to, for example, rates of cell proliferation, percentages of surviving cells, percentages of altered or modified sequences, levels of expression, levels of transcriptional or translational activity, and levels of enzymatic or protein activity, percentages of conversion of a cell of a first cell type to a cell of a second cell type, etc. It should be appreciated that the at least one component can comprise any component of the cell identity program including one or more components of the core regulatory circuitry or targets of autoregulated transcription factors expressed by the core regulatory circuitry. In some embodiments, the cell comprises a cell listed in Table 2 and the at least one component of the cell identity program comprises at least one component listed in Table 2 selected from the group consisting of (i) at least one gene encoding a master transcription factor, (ii) the master transcription factor encoded by the at least one gene, (iii) a target of the master transcription factor, (iv) at least one super-enhancer associated with any of (i)-(iii), or at least one component of the super-enhancer.

The methods for modulating cell identity contemplate modulating any or all components of the cell identity program of a particular cell or tissue. Generally, it is expected that the extent of modulation of any particular cell or tissue from a first type to a second type is proportionate to the number of components in the cell identity program modulated relative to the total number of components in the cell identity program. In some embodiments, the method comprises modulating at least two components, at least three components, at least four components, or at least five components, of the cell identity program in the cell. In some embodiments, the method comprises modulating at least 5%, at least 10%, at least 15%, at least 20%, at least 25%, at least 33%, at least 40%, or at least 50% of the components in the cell identity program. In some embodiments, the method comprises modulating at least 55%, at least 60%, at least 70%, at least 75%, at least 80%, or at least 90% of the components in the cell identity program of a cell. In some embodiments, the method comprises modulating 90%, 91%, 92%, 93%, 94%, 95%, 96%, 97%, 98%, 99%, or up to 100% of the components of the cell identity program of the cell.

In some embodiments, the method comprises modulating at least one component of the core regulatory circuitry in the cell, and at least one target of a master transcription factor in the core regulatory circuitry. In some embodiments, the method comprises modulating at least two components of the core regulatory circuitry in the cell and at least two targets of a master transcription factor in the core regulatory circuitry. In some embodiments, the method comprises modulating at least three components of the core regulatory circuitry in the cell and at least three targets of a master transcription factor in the core regulatory circuitry. In some embodiments, the method comprises modulating at least four components of the core regulatory circuitry in the cell and at least four targets of a master transcription factor in the core regulatory circuitry. In some embodiments, the method comprises modulating at least five components of the core regulatory circuitry in the cell and at least five targets of a master transcription factor in the core regulatory circuitry of the cell. In some embodiments, the method comprises modulating at least 6, at least 7, at least 8, at least 9, at least 10, at least 15, at least 20 or at least 25 components of the core regulatory circuitry in the cell and at least 6, at least 7, at least 8, at least 9, at least 10, at least 15, at least 20 or at least 25 targets of the master transcription factors in the core regulatory circuitry.

In some embodiments, the method comprises modulating all components of the core regulatory circuitry in the cell, and at least one target of a master transcription factor in the core regulatory circuitry. In some embodiments, the method comprises modulating at least one component of the core regulatory circuitry in the cell, and all of the targets of the master transcription factor in the core regulatory circuitry. In some embodiments, the method comprises modulating all components of the core regulatory circuitry in the cell. In some embodiments, the method comprises modulating all targets of master transcription factors in the core regulatory circuitry.

In some aspects, the disclosure relates to reprogramming cells of a first cell type to cells of a second cell type, e.g., to alter the identity of the cell of the first cell type. In some aspects, the disclosure provides a method of reprogramming a cell of a first cell type to a cell of a second cell type, the method comprising modulating at least one component of the core regulatory circuitry of the second cell type in the cell of the first cell type. In some aspects, the disclosure provides a method of reprogramming a cell of a first cell type to a cell of a second cell type, the method comprising modulating at least one component of the cell identity program of the second cell type in the cell of the first cell type. In some context, “modulating at least one component of the core regulatory circuitry and/or cell identity program” comprises activating the at least one component of the core regulatory circuitry and/or cell identity program, e.g., activating a transcriptional coactivator. Those skilled in the art will appreciate that activation of the at least one component of the core regulatory circuitry and/or cell identity program can be accomplished in a variety of ways, e.g., alone or in combination with conventional reprogramming methods. In some embodiments, activating the at least one component comprises expressing the at least one component of the core regulatory circuitry and/or cell identity program of the second cell type in the cell of the first type. Such expression can be accomplished using methods such as DNA transfection, for example transient transfection, mRNA transfection, viral infection, etc. It should be appreciated that expression of core regulatory circuitry for purposes of reprogramming can be conditional, e.g., inducible, e.g., under control of an inducible promoter, e.g., using an inducible expression system, e.g., Tet-On, Tet-Off. In some embodiments, activating the at least one component comprises introducing the at least one component of the core regulatory circuitry and/or cell identity program of the second cell type into the cell of the second type. For example, at least one component of the core regulatory circuitry and/or cell identity program of the second cell type, e.g., in polypeptide form, can be directly introduced into the cell of the first cell type. Such polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing. In some embodiments, activating the at least one component comprises contacting the cell with an agent that activates expression of the at least one component of the core regulatory circuitry and/or cell identity program of the second cell type in the cell of the first type. In some embodiments, activation of the at least one component of the core regulatory circuitry and/or cell identity program of the second cell type comprises any combination of the above methods.

In some context, “modulating at least one component of the core regulatory circuitry and/or cell identity program” comprises repressing the at least one component of the core regulatory circuitry and/or cell identity program. For example, if the at least one component of the core regulatory circuitry and/or cell identity program comprise a repressor, reducing the repressor's activity in the context of several other transcriptional activators, for example transiently, could result in activation of the core regulatory circuitry and/or cell identity program of the second cell type thereby reprogramming the cell. The disclosure contemplates any suitable method of repressing the at least one component of the core regulatory circuitry and/or cell identity program (e.g., transcriptional repressor). Exemplary methods of repressing the at least one component include contacting the cell or tissue with a dominant negative mutant of the transcriptional repressor, contacting the cell or tissue with a nucleic acid that inhibits transcription or translation of the transcriptional repressor, e.g., antisense oligonucleotides directed against the sequence encoding the transcriptional repressor or a regulatory element that drives expression of the transcriptional repressor, e.g., a super-enhancer or DNA sequence binding motif, shRNA, microRNA, aptamers, small molecule inhibitors that interfere with binding between the transcriptional repressor and a regulatory element, etc.

It should be appreciated that the extent of reprogramming of the cell from the first cell type to the cell of the second cell type is likely to increase proportionately the extent of core regulatory circuitry and/or cell identity program components of the cell of the second cell type activated in the cell of the first cell type. In other words, the more the activation profile of core regulatory circuitry and/or cell identity program components of the cell of the first type resembles the core regulatory circuitry and/or cell identity program of the cell of the second type, the more the cell of the first type will phenotypically resemble the cell of the second type, i.e., the reprogramming efficiency will increase with increased activation of the desired core regulatory circuitry and/or cell identity program components. For the avoidance of doubt, it should be appreciated that the expressions “activation profile” and “activation of the core regulatory circuitry and/or cell identity program” refer to the overall effect that modulation of the components of the core regulatory circuitry and/or cell identity programs have on the cell or tissue, taking into account the fact that both activating a transcriptional activator or coactivator and repressing or inhibiting a transcriptional repressor or corepressor result in an overall net effect that favors increased activity or activation of the core regulatory circuitry and/or cell identity program in such a way that the identity of the cell is reprogrammed from the cell of the first type to the cell of the second type as a result of such increased activity or activation. In some embodiments, modulating the at least one component of the core regulatory circuitry and/or cell identity program increases the overall activation or activity of the core transcriptional circuitry and/or cell identity program (e.g., by driving the expression of core transcriptional circuitry target genes) by at least 5%, 10%, 15%, 20%, 25%, 30%, 35%, 40%, 50%, 55%, 60%, 70%, 75%, 80%, 85%, 90%, or 95% or more. In some embodiments, modulating the at least one component of the core regulatory circuitry and/or cell identity program increases the overall activation or activity of the core transcriptional circuitry and/or cell identity program by at least 1.1 fold, 1.2 fold, 1.3 fold, 1.4 fold, 1.5 fold, 1.6 fold, 1.7 fold, 1.8 fold, 1.9 fold, 2.0 fold, 2.5 fold, 3 fold, 4 fold, 5 fold, 6 fold, 7 fold, 8 fold.

In some embodiments, at least two components, at least three components, at least four components, at least five components, at least six components, at least seven components, at least eight components, at least nine components, or at least ten components of the core regulatory circuitry and/or cell identity program of the second cell type are modulated (e.g., activated and/or repressed) in the cell of the first type. In some embodiments, at least 5%, at least 10%, at least 15%, at least 20%, at least 25%, at least 30%, at least 33%, at least 35%, at least 40%, at least 45%, at least 50% or more of the components of the core regulatory circuitry of the cell of the second type are modulated (e.g., activated and/or repressed) in the cell of the first type. In some embodiments, at least 55%, at least 60%, at least 65%, at least 70%, at least 75%, at least 80%, at least 85%, at least 87%, or at least 90% of the components of the core regulatory circuitry and/or cell identity program of the cell of the second type are modulated (e.g., activated and/or repressed) in the cell of the first type. In some embodiments, at least 91%, at least 92%, at least 93%, at least 94%, at least 95%, at least 96%, at least 97%, at least 98%, at least 99%, or 100% of the components of the core regulatory circuitry and/or cell identity program of the cell of the second type are modulated (e.g., activated and/or repressed) in the cell of the first type.

In some embodiments, modulating the at least one component of the core regulatory circuitry and/or cell identity program of the second cell type in the cell of the first type occurs ex vivo. In some embodiments, modulating the at least one component of the core regulatory circuitry and/or cell identity program of the second cell type in the cell of the first type occurs in vivo. In some embodiments, the method of reprogramming optionally comprises modulating (e.g., inhibiting) at least one component of the core regulatory circuitry and/or cell identity program of the first cell type.

It should be appreciated that the methods can be used to reprogram any cell of a first cell type to a cell of a second cell type as long as the core regulatory circuitry and/or cell identity program of the cell of the second cell type is known. In some embodiments, the cell of the first cell type comprises the core regulatory circuitry and/or cell identity program of a diseased cell, and the cell of the second cell type comprises the core regulatory circuitry and/or cell identity program of a normal cell. In some embodiments, the cell of the first cell type comprises the core regulatory circuitry and/or cell identity program of a terminally differentiated cell, and the cell of the second cell type comprises the core regulatory circuitry and/or cell identity program of a less differentiated cell. In some embodiments, the cell of the first cell type comprises the core regulatory circuitry and/or cell identity program of a first somatic cell type, and the cell of the second cell type comprises the core regulatory circuitry and/or cell identity program of a second somatic cell type. In some embodiments, the cell of the first cell type comprises the core regulatory circuitry and/or cell identity program of a somatic cell, and the cell of the second cell type comprises the core regulatory circuitry and/or cell identity program of an embryonic cell. In some embodiments, the cell of the first cell type comprises the core regulatory circuitry and/or cell identity program of a first tissue type, and the cell of the second type comprises the core regulatory circuitry and/or cell identity program of a second tissue type. In some embodiments, the cell of the first cell type comprises the core regulatory circuitry and/or cell identity program of a skin or fat cell, and the cell of the second cell type comprises the core regulatory circuitry and/or cell identity program of an internal cell or tissue. In some embodiments, the cell of the first cell type comprises the core regulatory circuitry and/or cell identity program of a tumor cell or tissue, and the cell of the second cell type comprises the core regulatory circuitry and/or cell identity program of a healthy cell or tissue.

In some embodiments, nucleic acids encoding one or more core regulatory circuitry components can be incorporated into a vector, which can be introduced into a cell whose reprogramming is desired. Accordingly, in some embodiments, the disclosure provides kits comprising at least one nucleic acid encoding a core regulatory circuitry component of a cell type of interest.

In some embodiments, reprogramming is effected without genetically modifying the cell being reprogrammed. In some embodiments, cells to be reprogrammed may be obtained from a patient (or donor, optionally one who is immunocompatible with the patient), reprogrammed ex vivo, and at least some of the resulting cells can be administered to the patient for purposes of cell-based therapy, e.g., regenerative medicine, e.g., restoring a degenerated, injured, damaged, or dysfunctional organ or tissue, cell-based immunotherapy (e.g., for cancer or an infection), or used to construct a tissue or organ ex vivo, which can be implanted into the patient. In some embodiments, the reprogrammed cells can optionally be expanded ex vivo prior to reprogramming, after reprogramming, or both.

In some aspects, the disclosure provides methods for determining a subset of core regulatory circuitry components for a cell or tissue that are sufficient to effect reprogramming of the cell or tissue, comprising systematically introducing all but a first, a second, a third, . . . up to an Nth (where N is an integer equal to the total number of core regulatory circuitry components for the cell or tissue) of the core regulatory circuitry components into the cell or tissue to be reprogrammed, and evaluating combinations of core regulatory circuitry components that are effective in reprogramming the cell or tissue.

The reprogramming methods described herein can be used for any purpose which would be desirable to a skilled person, e.g., use in cell therapy, e.g., autologous cell therapy. As an example, fibroblasts can be obtained from an individual and reprogrammed to muscle cells ex vivo for use in tissue repair. As another example, white fat can be reprogrammed to brown fat.

Aspects of the disclosure relate to diagnosing cell identity program-related disorders. As used herein a “cell identity program-related disorder” refers to any disease, condition, or disorder that is caused, correlated to, or associated with a deviation in sequence, expression, or activity of a component of a cell identity program in a cell or tissue, e.g., a diseased cell or tissue of interest, e.g., obtained from a subject suffering from any disease, condition, or disorder described herein. In some aspects, a method of diagnosing a cell identity program-related disorder comprising determining whether the cell identity program of the cell or tissue is enriched for disease-associated variations. Any suitable method can be used to determine enrichment of disease-associated variations in the cell identity program of a cell or tissue of interest. In some embodiments, determining whether the cell identity program of the cell or tissue is enriched for disease-associated variations comprises obtaining a sample comprising a cell or tissue of interest, and detecting the presence of disease-associated variations in components of the cell identity program of the cell or tissue of interest, wherein the cell identity program of the cell or tissue is enriched for disease-associated variations if at least two disease-associated variations are detected in the components of the cell identity program of the cell or tissue of interest.

Those skilled in the art will appreciate that the sensitivity and specificity of the diagnostic methods may increase as a function of the overall number of disease-associated variations detected in the cell identity program relative to the overall number of components in the cell identity program. In some embodiments, the cell identity program of the cell or tissue is enriched for disease-associated variations if at least three; at least four; at least five; or at least six disease associated variations are detected in the components of the cell identity program of the cell or tissue of interest. In some embodiments, the cell identity program of the cell or tissue is enriched for disease-associated variations if at least 7, at least 8, at least 9, or at least 10 disease-associated variations are detected in the components of the cell identity program. In some embodiments, the cell identity program of the cell or tissue is enriched for disease-associated variations if at least 5%, at least 6%, at least 7%, at least 8%, at least 9%, or at least 10% of the components of the cell identity program are determined to contain a disease-associated variation. In some embodiments, the cell identity program of the cell or tissue is enriched for disease-associated variations if at least 11%, at least 12%, at least 13%, at least 14%, at least 15%, at least 16%, at least 17%, at least 88%, at least 19%, at least 20%, at least 25% or more of the components of the cell identity program are determined to contain a disease-associated variation. In some embodiments, the cell identity program of the cell or tissue is enriched for disease-associated variations if at least 30%, at least 33%, at least 35%, at least 37%, at least 39%, at least 42%, at least 45%, at least 47%, at least 50%, at least 55%, at least 60% or more of the components of the cell identity program are determined to contain a disease-associated variation.

As used herein, the phrase “disease-associated variations” and “disease-associated variants” refers to variations in sequences, expression levels, or activity of components of a cell identity program in a particular cell or tissue of interest. In some embodiments, the disease associated variations comprise single nucleotide polymorphisms. In some embodiments, the disease-associated variations comprise GWAS variants. Any SNPs linked to a phenotypic trait or disease can be of use herein. In some embodiments, the SNP comprises one of more than 5,000 SNPs and diseases identified in more than 1,600 GWAS studies described in PCT International Application No. PCT/US2013/066957 (attorney docket no. WIBR-137-WO1), filed Oct. 25, 2013, the entirety of which is incorporated by reference herein.

In some embodiments, the disease-associated variations comprise GWAS variants in a super-enhancer associated with the core regulatory circuitry in the cell or tissue of interested selected from the group consisting of i) at least one gene encoding a master transcription factor, (ii) the master transcription factor encoded by the at least one gene, or (iii) at least one target of the master transcription factor. In some embodiments, the GWAS variant is selected from the group consisting of (i) a GWAS variant from Alzheimer disease present in the cell identity program of brain hippocampus; (ii) a GWAS variant from systemic lupus erythematosus present in the cell identity program of CD20 cells; (iii) a GWAS variant from fasting insulin trait present in the cell identity program of adipose nuclei; (iv) a GWAS variant from ulcerative colitis present in the cell identity program of sigmoid colon; (vi), a GWAS variant from electrocardiographic traits present in the cell identity program of left ventricle.

Aspects of the disclosure relate to various methods of treatment, e.g., treating cell identity program-related disorders. In some aspects, the disclosure provides a method of treating a cell identity program-related disorder in a subject in need thereof, comprising modulating at least one abnormal component of a cell identity program in a diseased cell or tissue of the subject. As used herein, “abnormal component” of a cell identity program refers to a component of a cell identity program which differs in sequence, expression and/or activity in the diseased cell or tissue compared to the sequence, expression or activity of the component in the corresponding healthy or normal cell or tissue. In some embodiments, modulating at least one abnormal component of the cell identity program in the diseased cell or tissue of the subject comprises administering to the subject an effective amount of an agent that modulates the at least one abnormal component of the cell identity program.

Aspects of the disclosure involve the use of agents. The disclosure contemplates the use of any agent that is suitable for a specified purpose, e.g. agents that modulate at least one component of a cell identity program, e.g., at least one abnormal component. Exemplary agents of use herein include, without limitation, small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, diseased cell or tissue comprises a tumor cell or tissue. In some embodiments, the diseased cell or tissue comprises a cell or tissue listed in Table 2, and the abnormal component comprises at least one component of the cell identity program of the cell listed in Table 2 selected from the group consisting of (i) a gene encoding a master transcription factor, (ii) the master transcription factor encoded by the gene, (iii) a target of the master transcription factor, (iv) a super-enhancer associated with any of (i)-(iii), or a component of the super-enhancer. In some embodiments, the method comprises diagnosing the subject as having the cell identity program-related disorder, e.g., according to a method described herein.

Aspects of the disclosure relate to identifying candidate modulators of core regulatory circuitry components of cells or tissues. Such candidate modulators can be useful, e.g., for reprogramming cells or tissues or treating diseases in which one or more components of the core regulatory circuitry comprises an abnormal component, e.g., the component comprises a disease-associated variant. In some aspects, the disclosure provides a method of identifying a candidate modulator of at least one component of the core regulatory circuitry of a cell or tissue, comprising: a) contacting a cell or tissue with a test agent; and b) assessing the ability of the test agent to modulate at least one component of the core regulatory circuitry of the cell or tissue, wherein the test agent is identified as a candidate modulator of the at least one component of the core regulatory circuitry of the cell or tissue if the at least one component of the core regulatory circuitry is activated or inhibited in the presence of the test agent. Activation or inhibition of the at least one component of the core regulatory circuitry can be measured by detecting and quantifying expression or activity of the at least one component of the core regulatory circuitry.

In some embodiments, the at least one component of the core regulatory circuitry of the cell or tissue comprises a reprogramming factor or a cell identity gene. In some embodiments, the at least one component of the core regulatory circuitry of the cell or tissue comprises a disease-associated variant.

In some aspects, the disclosure relates to methods of reprogramming cells comprising contacting the cells with candidate modulators identified according to the methods described herein. In some embodiments, at least one component of the core regulatory circuitry of the cell comprises a disease-associated variant. In some embodiments, contacting occurs in vivo or ex vivo.

Aspects of the disclosure relate to methods of identifying candidate modulators of cell identity program components in cells or tissue. In some aspects, the disclosure provides a method of identifying a candidate modulator of at least one component of the cell identity program of a cell or tissue, comprising: a) contacting a cell or tissue with a test agent; and b) assessing the ability of the test agent to modulate at least one component of the cell identity program of the cell or tissue, wherein the test agent is identified as a candidate modulator of the at least one component of the cell identity program of the cell or tissue if the at least one component of the cell identity program of the cell or tissue is activated or inhibited in the presence of the test agent. In some embodiments, the at least one component of the cell identity program of the cell or tissue comprises a reprogramming factor or a cell identity gene. In some embodiments, the at least one component of the cell identity program of the cell or tissue comprises a disease-associated variant.

In some aspects, the disclosure provides a method of reprogramming a cell comprising contacting the cell with the candidate modulator identified according to a method described herein. In some embodiments, at least one component of the core regulatory circuitry of the cell comprises a disease-associated variant. In some embodiments, contacting occurs in vivo or ex vivo.

Aspects of the disclosure relate to methods of identifying targets for drug discovery (e.g., cancer drug discovery). Such methods are useful for identifying core regulatory circuitry or cell identity programs of tumor cells or tissues which can be modulated in a way that shifts the tumor cells or tissues back towards the normal state, e.g., if a core regulatory circuitry component is overexpressed in tumor cells or tissue compared to normal cells or tissue, inhibiting its expression or activity in the tumor could shift the tumor cells or tissues back towards the normal state.

In some aspects, the disclosure provides, a method of identifying a target for drug discovery comprising identifying a variation in at least one component of the core regulatory circuitry of a cell or tissue that is more prevalent in subjects suffering from a disease than in healthy subjects, wherein the at least one component of the core regulatory circuitry of the cell or tissue that is more prevalent in subjects suffering from a disease than in healthy subjects comprises a disease-associated variant, and wherein the disease-associated variant is a target for drug discovery.

In some aspects, the disclosure provides a method of identifying a target for drug discovery comprising identifying a variation in at least one component of the cell identity program of a cell or tissue that is more prevalent in subjects suffering from a disease than in healthy subjects, wherein the at least one component of the cell identity program of the cell or tissue that is more prevalent in subjects suffering from a disease than in healthy subjects comprises a disease-associated variant, and wherein the disease-associated variant is a target for drug discovery.

In some embodiments, the target for drug discovery comprises a target for diagnostic purposes.

In some aspects, the disclosure provides a method of identifying a target for anti-cancer drug discovery comprising: a) comparing the core regulatory circuitry of a tumor cell or tissue with the core regulatory circuitry of a corresponding non-tumor cell or tissue; and b) identifying at least one component that differs between the core regulatory circuitry of the tumor cell or tissue and the corresponding non-tumor cell or tissue, wherein the at least one component that differs between the core regulatory circuitry of the tumor cell or tissue and the corresponding non-tumor cell or tissue is identified as a target for anti-cancer drug discovery. In some embodiments, a gene regulated by the at least one component is identified as a target for anti-cancer drug discovery. In some embodiments, the at least one component differs in sequence, expression, and/or activity.

In some aspects, the disclosure provides a method of identifying an anti-cancer agent comprising identifying a modulator of the target for anti-cancer drug discovery identified according to a method described herein.

In some aspects, the disclosure provides a method treating a cancer characterized by tumor cell or tissue comprising the target for anti-cancer drug discovery, comprising administering to a subject suffering from the cancer an effective amount of the anti-cancer agent identified according to a method described herein.

In some embodiments one or more steps of a method described herein is performed at least in part by a machine, e.g., computer (e.g., is computer-assisted) or other apparatus (device) or by a system comprising one or more computers or devices. “Computer-assisted” as used herein encompasses methods in which a computer is used to gather, process, manipulate, display, visualize, receive, transmit, store, or in any way handle or analyze information (e.g., data, results, structures, sequences, etc.). A method may comprise causing the processor of a computer to execute instructions to gather, process, manipulate, display, receive, transmit, or store data or other information. The instructions may be embodied in a computer program product comprising a computer-readable medium. A computer-readable medium may be any tangible medium (e.g., a non-transitory storage medium) having computer usable program instructions embodied in the medium. Any combination of one or more computer usable or computer readable medium(s) may be utilized in various embodiments. A computer-usable or computer-readable medium may be or may be part of, for example but not limited to, an electronic, magnetic, optical, electromagnetic, infrared, or semiconductor system, apparatus, or device. Examples of a computer-readable medium include, e.g., a hard disk, a random access memory (RAM), a read-only memory (ROM), an erasable programmable read-only memory (e.g., EPROM or Flash memory), a portable compact disc read-only memory (CDROM), a floppy disk, an optical storage device, or a magnetic storage device. In some embodiments a method comprises transmitting or receiving data or other information over a communication network. The data or information may be generated at or stored on a first computer-readable medium at a first location, transmitted over the communication network, and received at a second location, where it may be stored on a second computer-readable medium. A communication network may, for example, comprise one or more intranets or the Internet.

In some embodiments, a method of identifying the CRC and/or CIP may be embodied on a non-transitory computer-readable medium. In some embodiments, a CRC and/or CIP identified in accordance with the methods described herein may be embodied on a non-transitory computer-readable medium. In some embodiments a computer is used in sample tracking, data acquisition, and/or data management. For example, in some embodiments a sample ID is entered into a database stored on a computer-readable medium in association with a measurement or determination of a sequence, expression and/or activity. The sample ID may subsequently be used to retrieve a result of determining sequence, expression and/or activity in the sample. In some embodiments, automated image analysis of a sample is performed using appropriate software, comprising computer-readable instructions to be executed by a computer processor. For example, a program such as ImageJ (Rasband, W. S., ImageJ, U. S. National Institutes of Health, Bethesda, Md., USA, http://imagej.nih.gov/ij/, 1997-2012; Schneider, C. A., et al., Nature Methods 9: 671-675, 2012; Abramoff, M. D., et al., Biophotonics International, 11(7): 36-42, 2004) or others having similar functionality may be used. In some embodiments, an automated imaging system is used. In some embodiments an automated image analysis system comprises a digital slide scanner. In some embodiments the scanner acquires an image of a slide (e.g., following IHC for detection of a gene product) and, optionally, stores or transmits data representing the image. Data may be transmitted to a suitable display device, e.g., a computer monitor or other screen. In some embodiments an image or data representing an image is added to a patient medical record.

In some embodiments a machine, e.g., an apparatus or system, is adapted, designed, or programmed to perform an assay for measuring or determining sequence, expression or activity of a cell identity program component listed in Table 2. In some embodiments an apparatus or system may include one or more instruments (e.g., a PCR machine), an automated cell or tissue staining apparatus, a device that produces, records, or stores images, and/or one or more computer processors. The apparatus or system may perform a process using parameters that have been selected for detection and/or quantification of a gene product of master transcription factor listed in Table 2, e.g., in samples of tumor cells or tissue. The apparatus or system may be adapted to perform the assay on multiple samples in parallel and/or may comprise appropriate software to provide an interpretation of the result. The apparatus or system may comprise appropriate input and output devices, e.g., a keyboard, display, printer, etc. In some embodiments a slide scanning device such as those available from Aperio Technologies (Vista, Calif.), e.g., the ScanScope AT, ScanScope CS, or ScanScope FL or is used.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### Core Transcriptional Circuitries of Human Cells

### Introduction

The molecular pathways for cellular processes such as metabolism, energy production, and signal transduction have been described in some detail. In contrast, the transcriptional circuitries that control the gene expression programs that define cell identity have yet to be mapped in most cells. For such mapping, it is essential to identify the set of key transcription factors that are responsible for control of cell identity and to determine how they function together to regulate cell-type-specific gene expression programs.

The key transcription factors responsible for the control of embryonic stem cell identity have been identified and their genome-wide occupancy and functions have been investigated extensively. This small set of master transcription factors has been identified through genetic perturbation and by virtue of their ability to reprogram cells of various types into the pluripotent state characteristic of ESCs (Yamanaka and Blau, 2010; Hanna et al., 2010; Stadtfeld and Hochedlinger, 2010; Young, 2011). These ESC master transcription factors bind to clusters of enhancers, called super-enhancers, which drive the expression of genes encoding the master transcription factors themselves as well as other genes key to cell identity. The master transcription factors thus form an interconnected autoregulatory circuitry that is at the core of the transcriptional network and that controls the pluripotent gene expression program of ESCs. Little is known about the core transcriptional circuitries of most human cell types, but there has been considerable progress in identifying transcription factors that are essential for cell identity and cellular reprogramming in a number of cell types. For example, master transcription factors have been identified for various hematopoietic cells, hepatocytes, pancreatic islets, heart and neurons (Graf and Enver, 2009; Vierbuchen et al., Nature 2010; Zhou et al., Nature 2008; McCulley and Black, Curr Top Dev Biol 2012). These factors tend to share two features: (1) they are encoded by genes whose expression is driven by super-enhancers and (2) they bind their own SEs as well as those of other master TFs. We have used these two properties to create models of core transcriptional regulatory circuitries (CRCs) for a broad range of human cell types. We describe these CRCs, criteria that we used for initial validation, evidence that non-cancer disease-associated variation is concentrated in these CRCs, and how tumor cells can modify CRCs to produce oncogenic gene expression programs.

Results

Cell Identity Program Maps for Human Primary Cells and Tissues

To construct maps of the core regulatory circuitry (CRC) driving the cell identity program of human cell types, we used the logic outlined in FIG. 1. Detailed studies of the transcriptional control of cell identity in ESCs and a few other cell types have shown that master transcription factors—factors that dominate the control of the gene expression program that defines cell identity—are encoded by genes that are associated with super-enhancers (Hnisz et al., 2013). For 43 different human cell and tissue types, we first identified the set of genes encoding transcription factors that were associated with super-enhancers (FIG. 1A). We found that approximately 5% of the genes encoding TFs had super-enhancers in any one cell type. Importantly, the list of SE-associated TF genes correctly identified master TFs that had been previously described in six well-studied cell types (Table 1).

TABLE 1
Key transcription factors described in 6 different cell types.

| Cell Type | Factor | References |
|---|---|---|
| ESC | ESRRB | Ivanova et al., 2006; Zhou et al., 2007 |
| | KLF2 | Jiang et al. 2008 |
| | KLF4 | Takahashi and Yamanaka, 2006; Jiang et al. 2008 |
| | KLF5 | Ema et al., 2008; Jiang et al. 2008; Parisi et al., |
| | | 2008; |
| | LIN28 | Yu et al., 2007 |
| | NACC1/NAC1 | Kim et al., 2008 |
| | NANOG | Chambers et al., 2003; Mitsui et al., 2003 |
| | NR0B1/DAX1 | Niakan et al., 2006; Kim et al., 2008 |
| | NR5A2 | Gu et al., 2005; Zhou et al., 2007; Wang et al., 2011 |
| | POU5F1/OCT4 | Nichols et al., 1998; Niwa et al., 2000 |
| | PRDM14 | Tsuneyoshi et al., 2008; Chia et al., 2010 |
| | RARG | Wang et al., 2011 |
| | REST | Singh et al., 2008 |
| | SALL4 | Elling et al., 2006; Sakaki-Yumoto et al., 2006; Wu |
| | | et al., 2006; Zhang et al., 2006 |
| | SMAD1 | Chen et al., 2008 |
| | SOX2 | Avilion et al., 2003; Masui, et al., 2007 |
| | STAT3 | Boeuf et al., 1997; Niwa et al., 1998; Raz et al., |
| | | 1999 |
| | TBX3 | Ivanova et al., 2006 |
| | TCL1A | Ivanova et al., 2006; Matoba et al., 2006 |
| | UTF1 | Nishimoto et al., 2005; van den Boom et al., 2007 |
| | ZNF281/ZFP281 | Kim et al., 2008; Wang et al., 2008 |
| | E2F1 | Chen et al., 2008 |
| | MYC | Takahashi and Yamanaka, 2006; Kim et al., 2008 |
| | MYCN | Chen et al., 2008 |
| | REX1/ZFP42 | Zhang et al., 2006; Kim et al., 2008 |
| | ZFX | Galan-Caridad et al., 2007; Chen et al., 2008; Hu et |
| | | al., 2009 |
| Hepatocyte | HHEX | Keng et al., 2000; Martinez-Barbera et al., 2000; |
| | | Wallace et al., 2001 |
| | HNF4A | Parviz et al., 2003 |
| | ONECUT1/HNF6 | Clotman et al., 2002; Clotman et al., 2005; |
| | | Margagliotti et al., 2007 |
| | ONECUT2 | Clotman et al., 2005; Margagliotti et al., 2007 |
| | PROX1 | Sosa-Pineda et al., 2000; Kamiya et al., 2008; Seth |
| | | et al., 2014 |
| | TBX3 | Suzuki et al., 2008; Ludtke et al., 2009 |
| B-cell | BCL11A | Liu et al., 2003 |
| | EBF1 | Lin and Grosschedl, 1995; Lin et al., 2010 |
| | FOXO1 | Amin and Schlissel, 2008; Dengler et al., 2008; Lin |
| | | et al., 2010 |
| | IKZF1 | Georgopoulos et al., 1994 |
| | IKZF3 | Morgan et al., 1997; Wang et al., 1998 |
| | IRF4 | Lu et al., 2003; Ma et al., 2006 |
| | IRF8 | Lu et al., 2003; Ma et al., 2006 |
| | PAX5 | Urbanek et al., 1994; Nutt et al., 1999 |
| | POU2AF1/OCAB | Schubart et al., 1996; Kim et al., 1996; Nielsen et |
| | | al., 1996 |
| | RUNX1 | Seo et al., 2012; Niebuhr et al., 2013 |
| | SPI1/PU.1 | Scott et al., 1994 |
| | TCF3 | Lin et al., 2010 |
| | ZBTB7A/LRF | Maeda et al., 2007 |
| Pancreas | FOXA1/HNF3A | Kaestner et al., 1999; Shih et al., 1999 |
| | FOXA2/HNF3B | Sund et al., 2001; Lee et al., 2005 |
| | HES1 | Jensen et al., 2000; |
| | HHEX | Bort et al., 2004 |
| | INSM1 | Gierl et al., 2006; Mellitzer et al., 2006 |
| | ISL1 | Ahlgren et al., 1997 |
| | MAFA | Zhang et al., 2005; Zhou et al., 2008 |
| | MNX1/HB9 | Harrison et al., 1999 |
| | NEUROD1 | Naya et al., 1997 |
| | NEUROG3 | Apelqvist et al., 1999; Gradwohl et al., 2000; |
| | | Schwitzgebel et al., 2000; Zhou et al., 2008 |
| | NKX2-2 | Sussel et al., 1998 |
| | NKX6-1 | Sander et al., 1998; Lee et al., 2014; |
| | ONECUT1/HNF6 | Jacquemin et al., 2000; Jacquemin et al., 2003 |
| | PAX4 | Sosa-Pineda et al., 1997 |
| | PAX6 | St-Onge et al., 1997; Sander et al., 1997 |
| | PDX1 | Jonsson et al., 1994; Horb et al., 2003; Zhou et al., |
| | | 2008 |
| | PTF1A | Kawaguchi et al., 2002 |
| | RBPJ | Apelqvist et al., 1999 |
| | SOX9 | Lynn et al., 2007; Seymour et al., 2007 |
| Heart | FOXH1 | von Both et al., 2004 |
| | GATA4 | Grepin et al., 1997; Kuo et al., 1997; Molkentin et |
| | | al., 1997; Ieda et al., 2010 |
| | GATA5 | Reiter et al., 1999; Singh et al., 2010 |
| | GATA6 | Maitra et al., 2009 |
| | HAND2 | Srivastava et al., 1995 |
| | IRX4 | Bao et al., 1999; Bruneau et al., 2000 |
| | ISL1 | Cai et al., 2003; Lin et al., 2006 |
| | MEF2C | Srivastava et al., 1995; Lin et al., 1997; Ieda et al., |
| | | 2010 |
| | MYOCD | Wang et al., 2001; Nam et al., 2013 |
| | NKX2-5 | Lyons et al., 1995; Ieda et al., 1995 |
| | PITX2 | St. Amand et al., 1998; Logan et al., 1998; Ryan et |
| | | al., 1998 |
| | SRF | Parlakian et al., 2004 |
| | TBX1 | Vitelli et al., 2002; Xu et al., 2004 |
| | TBX2 | Christoffels et al., 2004 |
| | TBX3 | Hoogaars et al., 2004 |
| | TBX5 | Li et al., 1997; Basson et al., 1997; Ieda et al., 2010 |
| | TBX18 | Christoffels et al., 2006; Cai et al., 2008; Kapoor et |
| | | al., 2013 |
| | TBX20 | Stennard et al., 2003; Reim et al., 2005; Singh et al., |
| | | 2005; Stennard et al., 2005; Takeuchi et al., 2005; |
| | | Cai et al., 2005; Qian et al., 2005; Miskolczi- |
| | | McCallum et al., 2005; Brown et al., 2005 |
| Adipocyte | CEBPA | Freytag et al., 1994; Lin and Lane, 1994; Wang et |
| | | al., 1995 |
| | CEBPB | Yeh et al., 1995; Tanaka et al., 1997; Tang et al., |
| | | 2003; Ahfeldt et al., 2012 |
| | CEBPD | Yeh et al., 1995; Tanaka et al., 1997 |
| | CREB | Reusch et al., 2000; Zhang et al., 2004 |
| | EGR2/KROX20 | Chen et al., 2005 |
| | KLF4 | Birsoy et al., 2008 |
| | KLF5 | Oishi et al., 2005 |
| | KLF15 | Mori et al., 2005 |
| | LXR | Ross et al., 2002 |
| | NR3C1/GR | Yeh et al., 1995; Pantoja et al., 2008; Steger et al., |
| | | 2010 |
| | PPARG | Tontonoz et al., 1994; Egan et al |
| | PRDM16 | Seale et al., 2007; Seale et al., 2008 |
| | SREBF1 | Kim and Spiegelman, 1996 |
| | STAT5A | Nanbu-Wakao et al., 2002; Floyd and Stephens, |
| | | 2003; Shang and Waters, 2003 |
| | STAT5B | Nanbu-Wakao et al., 2002; Floyd and Stephens, |
| | | 2003 |

* Indicates transcription factor is part of the core regulatory circuitry

Previous studies have shown that master TFs bind their own enhancers (Lee and Young, 2013; Chen et al., 2008; Chew et al., 2005; Matoba et al., 2006), so we next identified the subset of SE-associated TF genes whose products were predicted to bind their own SEs (FIG. 1B). To do this, we carried out a motif search using FIMO (Find Individual Motif Occurrences) from the MEME (Multiple Em for Motif Elicitation) suite (Matys et al., 2006) to identify all occurrences of all the DNA sequence motifs within the TRANSFAC database. The recent identification of binding site sequences for >100 human TFs was critical for this approach (Jolma et al., 2013; Yan et al., 2013). We found that approximately 15% of the SE-associated TF genes had enhancer elements with DNA sequence motifs predicted for that TF (FIG. 2B). Importantly, when we compared the predicted binding sites of SE-associated TF genes with those actually bound based on ChIP-seq data (Garber et al., 2012; Gerstein et al., 2012; Yan et al., Cell 2013), we found that the vast majority of predictions were confirmed by the genome-wide binding data. We defined these SE-associated TF genes that were predicted to be bound by their own TFs as auto-regulated, as prior evidence in ESCs indicates that such genes are indeed autoregulated (see, e.g., Boyer et al., 2005).

In ESCs and a few other cell types, the master TFs bind to the enhancers of their own genes as well as those of other master TFs, forming an interconnected autoregulatory loop (Boyer et al., 2005; Odom et al., 2006; Lien et al., Dev Biol 2002; Novershtern et al., Cell 2011). This auto-regulatory loops form the core regulatory circuit of the cells identity program. We next identified the auto-regulated SE-associated TF genes encoding transcription factors that are also predicted to bind each of the super-enhancers of the other auto-regulated transcription factors, and assembled the largest fully inter-connected network of auto-regulated transcription factors (FIG. 1C). Importantly, the predicted map of interconnected autoregulatory circuitry for ESCs contained the TF genes and their interactions that have been described previously (Boyer et al., 2005; Whyte et al., 2013), but extended the predicted set of genes in the CRC to include MYB, FOXD3, NR5A1 and GTF2I. Previous studies have shown that FOXD3 is required for maintenance of pluripotent cells (Liu and Labosky, 2008; Calloni et al., 2013), and MYB and NR5A1 are involved in the control of development and differentiation (Fahl et al., 2009; Kolodziejska et al., 2008; Sakamoto et al., 2006; Melotti et al., 1996; Camats et al., 2012; Bashamboo et al., 2010).

To further define cell identity programs, we extended the concept that master TFs of ESCs bind the super-enhancers of key cell-type-specific genes that are expressed in these cells (Young, 2011; Lee and Young, 2013). We thus identified, for all cell types under study, all SE-associated genes whose SEs contained motifs for all of the transcription factors in the CRC (FIGS. 2A and 2B). The resultant cell identity programs thus contains an interconnected autoregulatory loop of TF genes and their products, together with a set of key SE-associated cell identity genes, as shown for the ESCs in FIG. 2C. In this example, the well-studied ESC master transcription factors Oct4, Sox2, Nanog, Esrrb, Klf4 (Whyte et al., 2013) were found in the CRC and other genes associated with pluripotency and ESC cell identity were found in the set of genes that were predicted to be targeted by the complete set of master factors of the CRC.

This approach allowed us to generate models of cell identity programs for 43 human primary cells and tissue types (Table 2).

Cell Identity Program Factors Cluster According to Known Lineages

During the course of development, cells evolve into different lineages which give rise to a specific panel of differentiated cell-types. The progressive differentiation of each cell type requires sequential activation or repression of transcriptional circuits, which have been especially well described for hematopoietic stem cell differentiation (Novershtern et al., Cell 2011; McArtur et al., 2009). We hypothesized that differentiated cell-types arising from the same developmental tissue would be more likely to share the same master transcription factors than cell-types originating from tissues which fate diverged earlier during development. To test this hypothesis, we carried out a hierarchical clustering analysis on the lists of factors we predicted to be part of the Cell Identity Program for each cell type. We obtained a dendrogram that remarkably recapitulated known lineage patterns (FIG. 2). Some transcription factors were exclusively shared by cell-types belonging to the same lineage, and were also predicted to be master transcription factors of progenitor cells of this lineage indicating that these transcription factors may be involved in inducing lineage determination.

CRC Master TFs have Binding Sites in Majority of Cell Identity Genes

In ESCs, the CRC master transcription factors occupy the enhancers of the majority of active cell identity genes (Kagey et al., 2010). We investigated whether the master transcription factors in the CRCs for the larger set of human cell types described here have binding site sequences in the enhancers of most active cell identity genes. The results show that this is indeed the case. Work described herein demonstrates that about 50% of the SE-associated genes in each cell-type have binding sites in their super-enhancer regulatory sequences for all the transcription factors in the CRC. Most of the known reprograming factors are either part of the CRC or the Cell Identity Program. We also observed that most of the cell identity genes have motifs in their regulatory sequences for at least one of the transcription factors of the CRC. These results suggest that the master TFs in the CRCs of most human cell types do indeed occupy the majority of active cell identity genes.

Cell Identity Programs are Enriched in Disease-Associated Sequence Variation

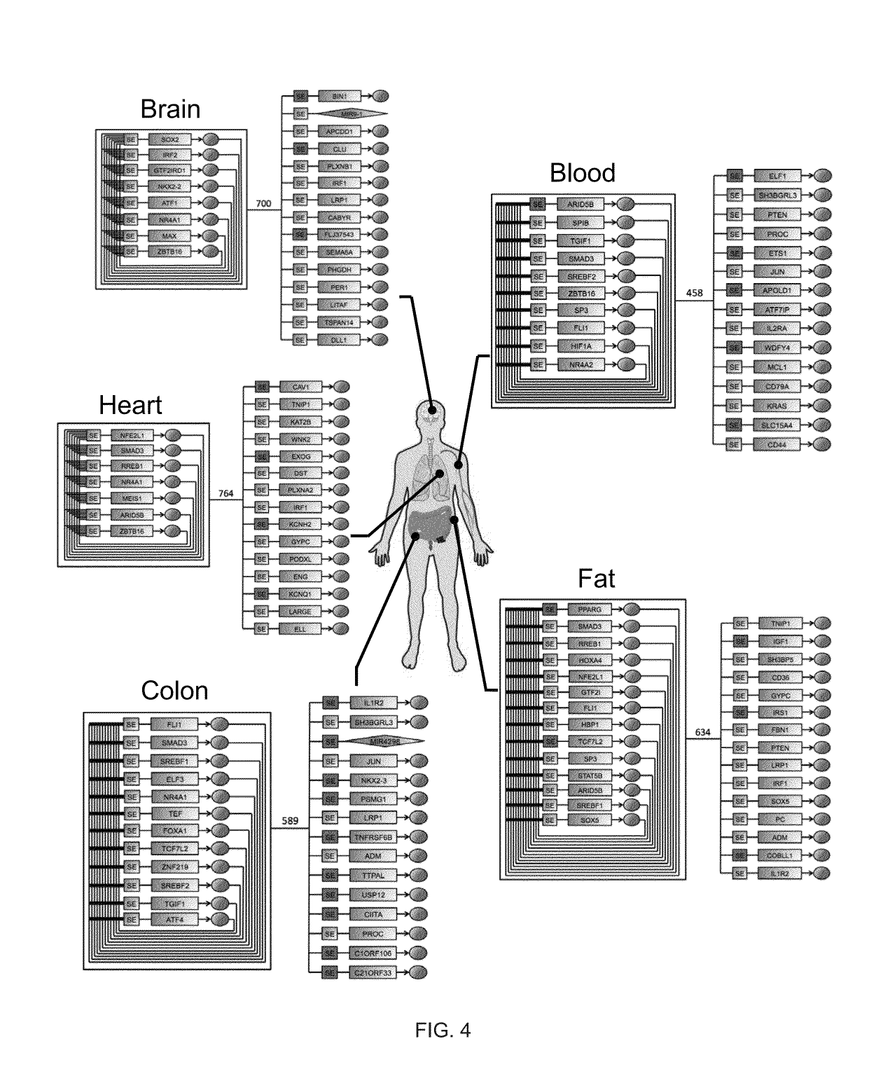

Work described herein demonstrates that the regulatory elements within the CRCs are enriched in disease-associated sequence variation (FIG. 4). DNA sequence variants have been found associated with human diseases and traits by genome-wide association studies (GWAS) (Hindroff et al., PNAS 2009). Most GWAS variants lie in non-coding regions of the genome and are enriched in regulatory regions (Maurano et al, Science 2012; Ernst et al, Nature 2011; Hnisz et al., Cell, 2013; Parker et al., PNAS 2013). The CRC models contain much of the super-enhancer associated GWAS variants.

Discussion

Work described herein provides the first maps of core regulatory circuitry of cell identity for a broad range of human cell types and tissues. These CRC maps provide founding models to test and expand knowledge of regulatory circuitry, provide guidance for reprogramming studies, and should facilitate understanding of disease causality.

Experimental Procedures

ChIP-seq Data

H3K27ac ChIP-seq sequence reads were either downloaded from GEO or generously shared by the NIH Roadmap Epigenome project (Bernstein et al., 2010) and were aligned to the hg19 version of the human genome using Bowtie 0.12.9 (Langmead et al., 2009) with parameters -k2-m2-n2-best.

CTC Mapper

During the course of work described herein an algorithm was developed to identify the transcriptional core circuitry of the cells which uses as input a file containing H3K27ac ChIP-seq reads aligned to the human genome together with its associated input ChIP-seq control aligned file, in a bam format. Briefly, super-enhancers and Master transcription Factors are identified using MACS 1.4.2 (Zhang et al., 2008) and ROSE (Loven et al., 2013) and a motif analysis is carried out on the super-enhancer constituent sequences extended 500 bp on each side using FIMO from the MEME suite (Matys et al., 2006). Interconnected auto-regulatory loops and their target genes are identified as described in the Experimental Procedures.

Lineage Clustering

Cell-type clustering based on core circuitry gene lists was done in R. A distance matrix was built based on the number of identical genes found in the cell type core circuitry gene lists on either all the genes in the core regulatory circuits or on the genes forming the interconnected autoregulatory loops only using the R dist function with euclidian method. The R hclust function with complete method was applied to the matrix of distances to generate the dendrograms.

GWAS Variant Analysis

Disease or trait-associated GWAS variants that had a dbSNP identifier and were found associated with the trait or disease in at least two independent studies were selected from the NHGRI (National Human Genome Research Institute) catalog of GWAS variants (www.genome.gov/gwastudies). Non-coding GWAS variants were identified as those that do not overlap with hg19 exonic regions. For each disease or trait, the GWAS variants were mapped to the super-enhancer regions identified in a cell-type relevant to the disease.

Identification of Super-Enhancers

First, super-enhancers are called as described in (Hnisz et al., 2013). Briefly, H3K27ac enriched regions are called using MACS 1.4.2 (Zhang et al., 2008) with parameters -p 1e-9 keep-dup=auto-w-S-space=50 on each H3K27ac ChIP-seq alignment and their corresponding input controls. ROSE (Loven et al., 2013) is then used to identify super-enhancers from the H3K27ac enriched regions. Briefly, H3K27ac enriched regions are considered as enhancers and are stitched together when they occur within 12.5 kb. In order to distinguish the H3K27ac enhancer signal from the H3K27ac promoter signal, constituent enhancers that are fully contained within 2 kb of a TSS are disregarded for stitching. Enhancer clusters that have a H3K27ac input-subtracted signal above a computed threshold defined by ranking the H3K27ac signal at enhancer clusters are identified as super-enhancers. Super-enhancers are then assigned to the closest active gene, considering the distance of the TSS to the center of the super-enhancers. We considered expressed the genes the first 2/3 genes based on their H3K27ac read density+−500 bp around their TSS rank. Genes called expressed using this metric show 90% overlap with genes having Gros-eq signal above background in their genes body (data not shown).

Identification of Master Transcription Factor Candidates

Super-enhancer-associated transcription factors are then selected from the lists of super-enhancer-associated genes using a list of transcription factors consisting in the concatenation of AnimaITFDB (Zhang et al., 2012), TcoF (Schaefer et al., 2011), Heinaniemi (ref) lists of factors. The super-enhancer-associated transcription factors are considered as the master transcription factor candidates for this cell type.

Motif Analysis

Super-enhancer constituent DNA sequences from all the identified super-enhancers in a given cell are extracted and extended 500 bp on each side to allow for transcription factor binding motif identification in and aside of H3K27ac peaks. A motif search is carried out on these sequences using FIMO (Find Individual Motif Occurrences) from the MEME (Multiple Em for Motif Elicitation) suite (Matys et al., 2006) to allow the identification of all occurrences of the DNA sequence motifs contained in a compiled library of motifs at a p-value threshold of 1e-4. The compiled library of motifs we used was composed of the TRANSFAC database motifs that we manually annotated to better associate the TRANSFAC motif designators with the official symbols, and the vertebrate motifs from the MEME database (updated on Jan. 23, 2014): (JASPAR CORE 2014 vertebrates (Mathelier et al., 2014), Jolma 2013 (Jolma et al., 2013), Homeodomains (Berger et al., 2008), mouse UniPROBE (Robasky et al., 2011), mouse and human ETS factors (Wei et al. 2010).

Identification of Interconnected Auto-Regulatory Loops and Associated Genes

The extended constituents that have motifs for each of the master transcription factor candidates are then identified and the official gene symbol of their associated genes is recovered using a dictionary associating each vertebrate to their associated gene official symbol or alias. From this list of genes, the transcription factors that have binding sites for their own protein products in their assigned extended super-enhancer constituents are defined as putative auto-regulated transcription factors. Interconnected auto-regulatory loops of the transcriptional core circuitry are then identified as the largest inter-connected network of auto-regulated transcription factors using an algorithm based on the identification of the maximum clique from the graph theory. Super-enhancer associated genes which contain binding motifs in their super-enhancer extended constituents for each of the predicted master transcription factors in the interconnected auto-regulatory loop are defined as target genes of the predicted master transcription factors. We calculated the pubmed (http://www.ncbi.nlm.nih.gov/pubmed) entry ratio of queries associating the gene official symbol or aliases in association with a list of terms related to the cell-type they were extracted from (Table 2) over the pubmed entries related to each factor only. For ease of representation, the 15 factors with the highest ratio were shown on the maps.

Transcription Factor Binding Predictions Validation

Oct4, Sox2 and Nanog ChIP-seq data were used to evaluate the predictions of the binding of transcription factors to super-enhancer extended constituent sequences. We identified the of super-enhancer constituents extended 500 bp on each side that had DNA motifs for each transcription factor and those that were overlapping with transcription factors binding sites as identified by the macs program ran on the ChIP-seq data with parameter -p 1e-9 keep-dup=auto-w-S-space=50. The true positive rates of transcription factor binding at super enhancer constituents was calculated by dividing the number motif containing super-enhancer constituent that are bound by the factors over the total number of motif containing super-enhancer constituents. Fold enrichments of true positive in super-enhancer sequences were next calculated by comparing the true positive rates at super-enhancers to the true positive rates obtained using a set of random genomic regions of the same size as the super-enhancer extended constituents.

GWAS Variant Enrichment Significance

Enrichment of the disease-associated GWAS variants in the super-enhancers of the core regulatory circuitry was calculated as the chance of capturing the same or a greater number of disease or trait-associated variants in a random set of genomic sequences, using a permutation test. A set of genomic sequences of the same size and originating from the same chromosome as each super-enhancer contained in the super-enhancer set of each relevant cell type was randomly selected 10000 times to calculate each empirical p-value.

TABLE 2
Models of cell identity programs for 43 human primary cells and tissue types.

| | [CRC transcription | CRC | | # Pubmed entries for factor | |
|---|---|---|---|---|---|
| | factors] # of | target | # Pubmed entries | associated to cell/tissue type | Ratio of |
| Cell/Tissue | CRC targets | genes | for the factor (A) | specific terms (B) | (B)/(A) |
| Astrocytes | [‘KLF12’- | ASB7 | 1 | 1 | 1 |
| | ‘GLIS3’- | ARHGAP23 | 3 | 2 | 0.666666667 |
| | ‘MEIS1’- | SYT14 | 5 | 3 | 0.6 |
| | ‘ZIC1’- | PHLDB1 | 25 | 14 | 0.56 |
| | ‘MYC’- | ZNF778 | 2 | 1 | 0.5 |
| | ‘TGIF1’- | SYNJ2 | 9 | 4 | 0.444444444 |
| | ‘HES1’- | NFIX | 56 | 24 | 0.428571429 |
| | ‘HIF1A’- | SEPT11 | 29 | 12 | 0.413793103 |
| | ‘FOXP1’]404 | HTR1D | 911 | 375 | 0.411635565 |
| | | TRAK1 | 21 | 8 | 0.380952381 |
| | | GAP43 | 1401 | 498 | 0.355460385 |
| | | PRICKLE2 | 31 | 11 | 0.35483871 |
| | | HOXA2 | 128 | 45 | 0.3515625 |
| | | STK40 | 194 | 65 | 0.335051546 |
| | | RTN4 | 3515 | 1169 | 0.33257468 |
| | | ELK3 | 304922 | 99651 | 0.326808167 |
| | | ADD3 | 100 | 32 | 0.32 |
| | | VIM | 1894 | 535 | 0.282470961 |
| | | COL4A2 | 7474 | 2054 | 0.274819374 |
| | | SCHIP1 | 15 | 4 | 0.266666667 |
| | | PTK7 | 956 | 241 | 0.25209205 |
| | | TGFBI | 2870 | 703 | 0.244947735 |
| | | ZFHX3 | 84 | 20 | 0.238095238 |
| | | MBNL2 | 42 | 10 | 0.238095238 |
| | | KCNA4 | 809 | 190 | 0.234857849 |
| | | MBP | 9274 | 2139 | 0.230644813 |
| | | RGS3 | 112 | 25 | 0.223214286 |
| | | KLF9 | 140 | 31 | 0.221428571 |
| | | CAPN2 | 115 | 25 | 0.217391304 |
| | | ZIC1 | 562 | 122 | 0.217081851 |
| | | PFKP | 42 | 9 | 0.214285714 |
| | | MIAT | 24 | 5 | 0.208333333 |
| | | ATXN1 | 1085 | 226 | 0.208294931 |
| | | NRP2 | 554 | 115 | 0.207581227 |
| | | TMEM30B | 10 | 2 | 0.2 |
| | | CDK17 | 5 | 1 | 0.2 |
| | | CPA1 | 5659 | 1130 | 0.199681923 |
| | | LPP | 1246 | 247 | 0.19823435 |
| | | NEDD9 | 511 | 99 | 0.193737769 |
| | | IER2 | 31 | 6 | 0.193548387 |
| | | FOSL2 | 260 | 50 | 0.192307692 |
| | | HES1 | 1584 | 303 | 0.191287879 |
| | | HIVEP2 | 100 | 19 | 0.19 |
| | | CALM2 | 58 | 11 | 0.189655172 |
| | | MAFK | 1466 | 276 | 0.188267394 |
| | | RAGE | 4126 | 726 | 0.175957344 |
| | | NAV1 | 2951 | 511 | 0.17316164 |
| | | NRP1 | 2030 | 346 | 0.17044335 |
| | | STARD13 | 53 | 9 | 0.169811321 |
| | | TGIF1 | 221 | 37 | 0.167420814 |
| BI_Adipose_Nuclei | [‘SOX5’, | CD36 | 183913 | 181760 | 0.988293378 |
| | ‘SREBF1’, | CIDEC | 102 | 93 | 0.911764706 |
| | ‘ARID5B’, | SREBF1 | 2637 | 2231 | 0.846037163 |
| | ‘STAT5B’, | LYRM1 | 10 | 8 | 0.8 |
| | ‘SP3’, | CIDEA | 125 | 95 | 0.76 |
| | ‘TCF7L2’, | ELOVL5 | 66 | 49 | 0.742424242 |
| | ‘SMAD3’, | LPL | 4894 | 3629 | 0.741520229 |
| | ‘HBP1’, | RFTN1 | 14 | 10 | 0.714285714 |
| | ‘PPARG’, | PTGER3 | 1158 | 815 | 0.703799655 |
| | ‘HOXA4’, | ADIPOR2 | 492 | 334 | 0.678861789 |
| | ‘RREB1’, | PPAP2B | 61 | 39 | 0.639344262 |
| | ‘NFE2L1’, | PPARG | 14509 | 8628 | 0.59466538 |
| | ‘GTF2I’, | APOL3 | 7 | 4 | 0.571428571 |
| | ‘FLI1’]634 | SLC27A3 | 27 | 15 | 0.555555556 |
| | | PIGV | 19 | 10 | 0.526315789 |
| | | TBC1D4 | 303 | 159 | 0.524752475 |
| | | PDK4 | 311 | 163 | 0.524115756 |
| | | ACACB | 205 | 105 | 0.512195122 |
| | | ZNF664 | 10 | 5 | 0.5 |
| | | MIR365-1 | 2 | 1 | 0.5 |
| | | C6orf106 | 2 | 1 | 0.5 |
| | | FABP4 | 3157 | 1565 | 0.495723788 |
| | | LY86-AS1 | 53 | 25 | 0.471698113 |
| | | EHBP1 | 15 | 7 | 0.466666667 |
| | | ALG9 | 26 | 12 | 0.461538462 |
| | | PLIN2 | 642 | 294 | 0.457943925 |
| | | LPIN2 | 40 | 18 | 0.45 |
| | | PGS1 | 41 | 18 | 0.43902439 |
| | | HRASLS2 | 7 | 3 | 0.428571429 |
| | | PLD1 | 502 | 215 | 0.428286853 |
| | | PIK3C2B | 109 | 45 | 0.412844037 |
| | | TMEM135 | 5 | 2 | 0.4 |
| | | GPAM | 570 | 216 | 0.378947368 |
| | | PCOLCE2 | 11 | 4 | 0.363636364 |
| | | CD180 | 121 | 44 | 0.363636364 |
| | | IRS1 | 2857 | 1004 | 0.351417571 |
| | | SEC14L1 | 18 | 6 | 0.333333333 |
| | | MGST1 | 231 | 77 | 0.333333333 |
| | | ATP8B4 | 3 | 1 | 0.333333333 |
| | | ARHGEF10L | 3 | 1 | 0.333333333 |
| | | IRS2 | 1446 | 470 | 0.325034578 |
| | | PHLDB2 | 16 | 5 | 0.3125 |
| | | ESYT2 | 13 | 4 | 0.307692308 |
| | | NRIP1 | 234 | 71 | 0.303418803 |
| | | MTMR2 | 96 | 29 | 0.302083333 |
| | | ENPP2 | 953 | 283 | 0.296956978 |
| | | TBX15 | 41 | 12 | 0.292682927 |
| | | PALMD | 7 | 2 | 0.285714286 |
| | | FNDC3B | 21 | 6 | 0.285714286 |
| | | GPR116 | 15 | 4 | 0.266666667 |
| BI_Brain_Angular_Gyrus | [‘SOX2’, | PLEKHG3 | 2 | 2 | 1 |
| | ‘SREBF1’, | LRRTM2 | 16 | 16 | 1 |
| | ‘TCF12’, | LOC286094 | 1 | 1 | 1 |
| | ‘MAX’]507 | ANKRD43 | 1 | 1 | 1 |
| | | CAMK2A | 181 | 151 | 0.834254144 |
| | | NEURL | 12 | 10 | 0.833333333 |
| | | KCNK7 | 5 | 4 | 0.8 |
| | | DPYSL2 | 344 | 274 | 0.796511628 |
| | | MAP1B | 585 | 450 | 0.769230769 |
| | | SLC1A3 | 1071 | 818 | 0.763772176 |
| | | POMT2 | 68 | 50 | 0.735294118 |
| | | ADAP1 | 41 | 30 | 0.731707317 |
| | | SORT1 | 589 | 418 | 0.709677419 |
| | | PEX5L | 44 | 31 | 0.704545455 |
| | | DSCAML1 | 13 | 9 | 0.692307692 |
| | | TTC7B | 3 | 2 | 0.666666667 |
| | | TMCC2 | 3 | 2 | 0.666666667 |
| | | TECPR2 | 3 | 2 | 0.666666667 |
| | | KCTD7 | 12 | 8 | 0.666666667 |
| | | ARHGAP23 | 3 | 2 | 0.666666667 |
| | | TUBA1A | 95 | 61 | 0.642105263 |
| | | TTYH1 | 13 | 8 | 0.615384615 |
| | | LINGO1 | 104 | 64 | 0.615384615 |
| | | SRGAP2 | 66 | 40 | 0.606060606 |
| | | SLC6A1 | 509 | 306 | 0.601178782 |
| | | C18orf1 | 5 | 3 | 0.6 |
| | | ANK3 | 248 | 148 | 0.596774194 |
| | | FXYD6 | 24 | 14 | 0.583333333 |
| | | UNC5C | 85 | 49 | 0.576470588 |
| | | GPR56 | 95 | 54 | 0.568421053 |
| | | FEZ1 | 85 | 48 | 0.564705882 |
| | | SYNJ2 | 9 | 5 | 0.555555556 |
| | | CDK18 | 47 | 26 | 0.553191489 |
| | | PHLDB1 | 25 | 13 | 0.52 |
| | | NCAM1 | 13560 | 6868 | 0.506489676 |
| | | ZNF778 | 2 | 1 | 0.5 |
| | | ZNF536 | 2 | 1 | 0.5 |
| | | TMEM144 | 2 | 1 | 0.5 |
| | | PHYHIPL | 2 | 1 | 0.5 |
| | | PCDH1 | 34 | 17 | 0.5 |
| | | GNAZ | 64 | 32 | 0.5 |
| | | CPNE2 | 18 | 9 | 0.5 |
| | | CORO2B | 2 | 1 | 0.5 |
| | | MOBP | 71 | 35 | 0.492957746 |
| | | GPRC5B | 21 | 10 | 0.476190476 |
| | | POU3F3 | 55 | 26 | 0.472727273 |
| | | UNC5B | 109 | 51 | 0.467889908 |
| | | GNG7 | 11 | 5 | 0.454545455 |
| | | NFIX | 56 | 25 | 0.446428571 |
| | | GPR37L1 | 9 | 4 | 0.444444444 |
| BI_Brain_Anterior_Caudate | [‘IRF2’, | TTLL11 | 1 | 1 | 1 |
| | ‘MAX’, | PLEKHG3 | 2 | 2 | 1 |
| | ‘ZBTB16’, | PGBD5 | 1 | 1 | 1 |
| | ‘SOX2’, | LRRTM2 | 16 | 16 | 1 |
| | ‘NR4A1’, | HMP19 | 1 | 1 | 1 |
| | ‘TCF12’, | ANKRD43 | 1 | 1 | 1 |
| | ‘DBP’]677 | FLRT1 | 5 | 4 | 0.8 |
| | | DPYSL2 | 344 | 274 | 0.796511628 |
| | | GRIN2C | 420 | 326 | 0.776190476 |
| | | MAP1B | 585 | 450 | 0.769230769 |
| | | SLC1A3 | 1071 | 818 | 0.763772176 |
| | | NPAS3 | 36 | 27 | 0.75 |
| | | KIAA1147 | 4 | 3 | 0.75 |
| | | POMT2 | 68 | 50 | 0.735294118 |
| | | ADAP1 | 41 | 30 | 0.731707317 |
| | | SORT1 | 589 | 418 | 0.709677419 |
| | | PEX5L | 44 | 31 | 0.704545455 |
| | | DSCAML1 | 13 | 9 | 0.692307692 |
| | | TTC7B | 3 | 2 | 0.666666667 |
| | | TMCC2 | 3 | 2 | 0.666666667 |
| | | OPALIN | 15 | 10 | 0.666666667 |
| | | KCTD7 | 12 | 8 | 0.666666667 |
| | | ARHGAP23 | 3 | 2 | 0.666666667 |
| | | TUBA1A | 95 | 61 | 0.642105263 |
| | | SLC24A2 | 50 | 32 | 0.64 |
| | | SLC6A9 | 339 | 215 | 0.634218289 |
| | | CTNND2 | 49 | 30 | 0.612244898 |
| | | SRGAP2 | 66 | 40 | 0.606060606 |
| | | SLC6A1 | 509 | 306 | 0.601178782 |
| | | C18orf1 | 5 | 3 | 0.6 |
| | | ANK3 | 248 | 148 | 0.596774194 |
| | | PLXND1 | 37 | 22 | 0.594594595 |
| | | PCDH9 | 32 | 19 | 0.59375 |
| | | UNC5C | 85 | 49 | 0.576470588 |
| | | KIAA0319L | 7 | 4 | 0.571428571 |
| | | GPR56 | 95 | 54 | 0.568421053 |
| | | FEZ1 | 85 | 48 | 0.564705882 |
| | | SYNJ2 | 9 | 5 | 0.555555556 |
| | | PITPNM2 | 18 | 10 | 0.555555556 |
| | | CDK18 | 47 | 26 | 0.553191489 |
| | | SYT11 | 20 | 11 | 0.55 |
| | | TUBB4 | 17 | 9 | 0.529411765 |
| | | PHLDB1 | 25 | 13 | 0.52 |
| | | ARNT2 | 97 | 50 | 0.515463918 |
| | | ZSWIM6 | 2 | 1 | 0.5 |
| | | ZNF536 | 2 | 1 | 0.5 |
| | | ZC3H4 | 2 | 1 | 0.5 |
| | | TMEM144 | 2 | 1 | 0.5 |
| | | PHYHIPL | 2 | 1 | 0.5 |
| | | PCDH1 | 34 | 17 | 0.5 |
| BI_Brain_Cingulate_Gyrus | [‘IRF2’, | PLEKHG3 | 2 | 2 | 1 |
| | ‘ARID5B’, | PGBD5 | 1 | 1 | 1 |
| | ‘ZBTB16’, | LRRTM2 | 16 | 16 | 1 |
| | ‘NKX2-2’, | FAM19A5 | 4 | 4 | 1 |
| | ‘SOX2’, | CLEC2L | 1 | 1 | 1 |
| | ‘MAX’, | NTRK2 | 3514 | 3233 | 0.920034149 |
| | ‘NR4A1’, | NEURL | 12 | 10 | 0.833333333 |
| | ‘ATF1’]712 | DLG2 | 144 | 116 | 0.805555556 |
| | | OLIG1 | 158 | 127 | 0.803797468 |
| | | FLRT1 | 5 | 4 | 0.8 |
| | | DPYSL2 | 344 | 274 | 0.796511628 |
| | | C19orf12 | 23 | 18 | 0.782608696 |
| | | MAP1B | 585 | 450 | 0.769230769 |
| | | SLC1A3 | 1071 | 818 | 0.763772176 |
| | | NPAS3 | 36 | 27 | 0.75 |
| | | KIAA1147 | 4 | 3 | 0.75 |
| | | POMT2 | 68 | 50 | 0.735294118 |
| | | PEX5L | 44 | 31 | 0.704545455 |
| | | MDGA1 | 20 | 14 | 0.7 |
| | | DSCAML1 | 13 | 9 | 0.692307692 |
| | | TTC7B | 3 | 2 | 0.666666667 |
| | | TMCC2 | 3 | 2 | 0.666666667 |
| | | TECPR2 | 3 | 2 | 0.666666667 |
| | | OPALIN | 15 | 10 | 0.666666667 |
| | | NKAIN1 | 3 | 2 | 0.666666667 |
| | | KCTD7 | 12 | 8 | 0.666666667 |
| | | ARHGAP23 | 3 | 2 | 0.666666667 |
| | | TUBA1A | 95 | 61 | 0.642105263 |
| | | SLC24A2 | 50 | 32 | 0.64 |
| | | SLC6A9 | 339 | 215 | 0.634218289 |
| | | SH3GL3 | 19 | 12 | 0.631578947 |
| | | TRIM2 | 13 | 8 | 0.615384615 |
| | | SRGAP2 | 66 | 40 | 0.606060606 |
| | | SLC6A1 | 509 | 306 | 0.601178782 |
| | | NINJ2 | 15 | 9 | 0.6 |
| | | C18orf1 | 5 | 3 | 0.6 |
| | | ANK3 | 248 | 148 | 0.596774194 |
| | | PLXND1 | 37 | 22 | 0.594594595 |
| | | PCDH9 | 32 | 19 | 0.59375 |
| | | UNC5C | 85 | 49 | 0.576470588 |
| | | GLTSCR1 | 7 | 4 | 0.571428571 |
| | | GPR56 | 95 | 54 | 0.568421053 |
| | | CADM4 | 23 | 13 | 0.565217391 |
| | | FEZ1 | 85 | 48 | 0.564705882 |
| | | SYNJ2 | 9 | 5 | 0.555555556 |
| | | APBB2 | 33 | 18 | 0.545454545 |
| | | TUBB4 | 17 | 9 | 0.529411765 |
| | | PHLDB1 | 25 | 13 | 0.52 |
| | | NKX2-2 | 319 | 162 | 0.507836991 |
| | | NCAM1 | 13560 | 6868 | 0.506489676 |
| BI_Brain_Hippocampus_Middle | [‘IRF2’, | PLEKHG3 | 2 | 2 | 1 |
| | ‘ZBTB16’, | PGBD5 | 1 | 1 | 1 |
| | ‘MAX’, | LRRTM2 | 16 | 16 | 1 |
| | ‘NR4A1’, | LENG8 | 1 | 1 | 1 |
| | ‘SOX2’, | FAM19A5 | 4 | 4 | 1 |
| | ‘ATF1’, | CCDC85C | 1 | 1 | 1 |
| | ‘GTF2IRD1’, | ZIC5 | 23 | 21 | 0.913043478 |
| | ‘NKX2-2’]700 | NEURL | 12 | 10 | 0.833333333 |
| | | OLIG1 | 158 | 127 | 0.803797468 |
| | | FLRT1 | 5 | 4 | 0.8 |
| | | DPYSL2 | 344 | 274 | 0.796511628 |
| | | C19orf12 | 23 | 18 | 0.782608696 |
| | | MAP1B | 585 | 450 | 0.769230769 |
| | | POMT2 | 68 | 50 | 0.735294118 |
| | | SORT1 | 589 | 418 | 0.709677419 |
| | | PEX5L | 44 | 31 | 0.704545455 |
| | | NLGN3 | 47 | 33 | 0.70212766 |
| | | MDGA1 | 20 | 14 | 0.7 |
| | | DSCAML1 | 13 | 9 | 0.692307692 |
| | | TTC7B | 3 | 2 | 0.666666667 |
| | | TMCC2 | 3 | 2 | 0.666666667 |
| | | TECPR2 | 3 | 2 | 0.666666667 |
| | | OPALIN | 15 | 10 | 0.666666667 |
| | | KCTD7 | 12 | 8 | 0.666666667 |
| | | ARHGAP23 | 3 | 2 | 0.666666667 |
| | | ZIC4 | 37 | 24 | 0.648648649 |
| | | SLC6A9 | 339 | 215 | 0.634218289 |
| | | TRIM2 | 13 | 8 | 0.615384615 |
| | | SLC6A1 | 509 | 306 | 0.601178782 |
| | | NINJ2 | 15 | 9 | 0.6 |
| | | C18orf1 | 5 | 3 | 0.6 |
| | | ANK3 | 248 | 148 | 0.596774194 |
| | | PLXND1 | 37 | 22 | 0.594594595 |
| | | UNC5C | 85 | 49 | 0.576470588 |
| | | GPR56 | 95 | 54 | 0.568421053 |
| | | FEZ1 | 85 | 48 | 0.564705882 |
| | | NINJ1 | 57 | 32 | 0.561403509 |
| | | SYNJ2 | 9 | 5 | 0.555555556 |
| | | NTNG2 | 44 | 24 | 0.545454545 |
| | | HCN2 | 376 | 203 | 0.539893617 |
| | | TUBB4 | 17 | 9 | 0.529411765 |
| | | PHLDB1 | 25 | 13 | 0.52 |
| | | ARNT2 | 97 | 50 | 0.515463918 |
| | | MCF2L | 6927 | 3526 | 0.509022665 |
| | | NKX2-2 | 319 | 162 | 0.507836991 |
| | | NCAM1 | 13560 | 6868 | 0.506489676 |
| | | ZNF778 | 2 | 1 | 0.5 |
| | | ZNF536 | 2 | 1 | 0.5 |
| | | ZC3H4 | 2 | 1 | 0.5 |
| | | TMEM144 | 2 | 1 | 0.5 |
| BI_Brain_Inferior_Temporal_Lobe | [‘NR4A1’, | TTLL11 | 1 | 1 | 1 |
| | ‘TCF12’, | PLEKHG3 | 2 | 2 | 1 |
| | ‘SOX2’, | PGBD5 | 1 | 1 | 1 |
| | ‘ZBTB16’, | LRRTM2 | 16 | 16 | 1 |
| | ‘SREBF2’, | LOC286094 | 1 | 1 | 1 |
| | ‘MAX’, | FAM131B | 1 | 1 | 1 |
| | ‘ARID5B’]804 | NTRK2 | 3514 | 3233 | 0.920034149 |
| | | CAMK2A | 181 | 151 | 0.834254144 |
| | | NEURL | 12 | 10 | 0.833333333 |
| | | DLG2 | 144 | 116 | 0.805555556 |
| | | OLIG1 | 158 | 127 | 0.803797468 |
| | | FLRT1 | 5 | 4 | 0.8 |
| | | DPYSL2 | 344 | 274 | 0.796511628 |
| | | NRXN2 | 13 | 10 | 0.769230769 |
| | | MAP1B | 585 | 450 | 0.769230769 |
| | | SLC1A3 | 1071 | 818 | 0.763772176 |
| | | RTN4RL1 | 21 | 16 | 0.761904762 |
| | | KIAA1147 | 4 | 3 | 0.75 |
| | | POMT2 | 68 | 50 | 0.735294118 |
| | | SORT1 | 589 | 418 | 0.709677419 |
| | | PEX5L | 44 | 31 | 0.704545455 |
| | | DSCAML1 | 13 | 9 | 0.692307692 |
| | | TTC7B | 3 | 2 | 0.666666667 |
| | | TMCC2 | 3 | 2 | 0.666666667 |
| | | TECPR2 | 3 | 2 | 0.666666667 |
| | | OPALIN | 15 | 10 | 0.666666667 |
| | | KCTD7 | 12 | 8 | 0.666666667 |
| | | ARHGAP23 | 3 | 2 | 0.666666667 |
| | | SORCS2 | 17 | 11 | 0.647058824 |
| | | TUBA1A | 95 | 61 | 0.642105263 |
| | | SLC24A2 | 50 | 32 | 0.64 |
| | | LINGO1 | 104 | 64 | 0.615384615 |
| | | CTNND2 | 49 | 30 | 0.612244898 |
| | | SLC6A1 | 509 | 306 | 0.601178782 |
| | | NINJ2 | 15 | 9 | 0.6 |
| | | C18orf1 | 5 | 3 | 0.6 |
| | | ANK3 | 248 | 148 | 0.596774194 |
| | | PCDH9 | 32 | 19 | 0.59375 |
| | | FXYD6 | 24 | 14 | 0.583333333 |
| | | KCNC4 | 130 | 75 | 0.576923077 |
| | | UNC5C | 85 | 49 | 0.576470588 |
| | | GLTSCR1 | 7 | 4 | 0.571428571 |
| | | GPR56 | 95 | 54 | 0.568421053 |
| | | CADM4 | 23 | 13 | 0.565217391 |
| | | FEZ1 | 85 | 48 | 0.564705882 |
| | | KCTD1 | 2421 | 1364 | 0.563403552 |
| | | SYNJ2 | 9 | 5 | 0.555555556 |
| | | PITPNM2 | 18 | 10 | 0.555555556 |
| | | CDK18 | 47 | 26 | 0.553191489 |
| | | SYT11 | 20 | 11 | 0.55 |
| BI_Brain_Mid_Frontal_Lobe | [‘SOX2’, | PLEKHG3 | 2 | 2 | 1 |
| | ‘NR4A1’, | PCDHGC5 | 1 | 1 | 1 |
| | ‘ZBTB16’, | C14orf23 | 2 | 2 | 1 |
| | ‘TEF’]227 | DPYSL2 | 344 | 274 | 0.796511628 |
| | | MAP1A | 134 | 99 | 0.73880597 |
| | | POMT2 | 68 | 50 | 0.735294118 |
| | | SORT1 | 589 | 418 | 0.709677419 |
| | | DSCAML1 | 13 | 9 | 0.692307692 |
| | | TMCC2 | 3 | 2 | 0.666666667 |
| | | SRGAP2 | 66 | 40 | 0.606060606 |
| | | FEZ1 | 85 | 48 | 0.564705882 |
| | | SYNJ2 | 9 | 5 | 0.555555556 |
| | | PITPNM2 | 18 | 10 | 0.555555556 |
| | | CDK18 | 47 | 26 | 0.553191489 |
| | | PHLDB1 | 25 | 13 | 0.52 |
| | | PHYHIPL | 2 | 1 | 0.5 |
| | | PCDH1 | 34 | 17 | 0.5 |
| | | CPNE2 | 18 | 9 | 0.5 |
| | | CORO2B | 2 | 1 | 0.5 |
| | | GPRC5B | 21 | 10 | 0.476190476 |
| | | POU3F3 | 55 | 26 | 0.472727273 |
| | | GNG7 | 11 | 5 | 0.454545455 |
| | | NFIX | 56 | 25 | 0.446428571 |
| | | ADORA1 | 4941 | 2107 | 0.426431896 |
| | | PLLP | 43 | 18 | 0.418604651 |
| | | RTN4 | 3515 | 1418 | 0.40341394 |
| | | NAV1 | 2951 | 1173 | 0.397492375 |
| | | SCARB2 | 1431 | 559 | 0.390635919 |
| | | SOX2 | 3476 | 1159 | 0.333429229 |
| | | RTDR1 | 3 | 1 | 0.333333333 |
| | | ITPK1-AS1 | 12 | 4 | 0.333333333 |
| | | HMG20A | 15 | 5 | 0.333333333 |
| | | MEF2D | 168 | 51 | 0.303571429 |
| | | COBL | 47 | 14 | 0.29787234 |
| | | ZMYND8 | 11 | 3 | 0.272727273 |
| | | CELSR2 | 67 | 18 | 0.268656716 |
| | | SCHIP1 | 15 | 4 | 0.266666667 |
| | | MBNL2 | 42 | 11 | 0.261904762 |
| | | ITPKB | 54 | 14 | 0.259259259 |
| | | STMN4 | 209 | 53 | 0.253588517 |
| | | MAP6D1 | 4 | 1 | 0.25 |
| | | KLF9 | 140 | 33 | 0.235714286 |
| | | MBP | 9274 | 2176 | 0.234634462 |
| | | MALAT1 | 2222 | 507 | 0.228172817 |
| | | NFIB | 1060 | 233 | 0.219811321 |
| | | PICK1 | 9417 | 2020 | 0.214505681 |
| | | FMNL2 | 24 | 5 | 0.208333333 |
| | | NR2F1 | 488 | 98 | 0.200819672 |
| | | HIP1R | 85 | 17 | 0.2 |
| | | BIN1 | 225 | 45 | 0.2 |
| BI_CD34_Primary_RO01480 | [‘FOXP1’, | ZNF445 | 1 | 1 | 1 |
| | ‘IKZF1’, | TMEM140 | 1 | 1 | 1 |
| | ‘RREB1’, | INO80D | 1 | 1 | 1 |
| | ‘NFE2’, | C10orf107 | 4 | 4 | 1 |
| | ‘STAT5A’, | PROM1 | 3635 | 3338 | 0.91829436 |
| | ‘CTCF’, | CD34 | 26251 | 20393 | 0.776846596 |
| | ‘TGIF1’]287 | RNLS | 82 | 61 | 0.743902439 |
| | | CLEC9A | 39 | 29 | 0.743589744 |
| | | ICAM2 | 316 | 222 | 0.702531646 |
| | | ITGA4 | 2169 | 1465 | 0.675426464 |
| | | MIR326 | 12 | 8 | 0.666666667 |
| | | PTPRC | 17928 | 11944 | 0.666220437 |
| | | APOA1 | 1088 | 717 | 0.659007353 |
| | | GATA2 | 856 | 540 | 0.630841121 |
| | | MSI2 | 51 | 32 | 0.62745098 |
| | | LMO2 | 440 | 273 | 0.620454545 |
| | | TBCC | 2718 | 1639 | 0.603016924 |
| | | ZNF521 | 25 | 15 | 0.6 |
| | | MIR142 | 69 | 40 | 0.579710145 |
| | | CD53 | 152 | 87 | 0.572368421 |
| | | SELL | 10547 | 5847 | 0.554375652 |
| | | CD97 | 152 | 80 | 0.526315789 |
| | | RUNX1 | 3237 | 1619 | 0.500154464 |
| | | KIAA0247 | 4 | 2 | 0.5 |
| | | MEIS1 | 322 | 160 | 0.49689441 |
| | | LCP1 | 5361 | 2637 | 0.491885842 |
| | | MIR223 | 315 | 151 | 0.479365079 |
| | | AKNA | 11 | 5 | 0.454545455 |
| | | AKAP13 | 3329 | 1481 | 0.444878342 |
| | | LYN | 2247 | 960 | 0.427236315 |
| | | MAT2B | 818 | 348 | 0.425427873 |
| | | STAT5A | 4961 | 2103 | 0.42390647 |
| | | LPXN | 26 | 11 | 0.423076923 |
| | | CD164 | 219 | 92 | 0.420091324 |
| | | LAPTM5 | 31 | 13 | 0.419354839 |
| | | UNK | 575 | 240 | 0.417391304 |
| | | MBP | 9274 | 3844 | 0.414492129 |
| | | ELF1 | 109 | 45 | 0.412844037 |
| | | B2M | 671 | 274 | 0.408345753 |
| | | IKZF1 | 1278 | 469 | 0.366979656 |
| | | STK17B | 42 | 15 | 0.357142857 |
| | | IER2 | 31 | 11 | 0.35483871 |
| | | MYCT1 | 32 | 11 | 0.34375 |
| | | FBRS | 7909 | 2709 | 0.342521178 |
| | | RALGDS | 1262 | 428 | 0.339144216 |
| | | ZFP36 | 9123 | 3089 | 0.33859476 |
| | | HNRNPK | 205 | 69 | 0.336585366 |
| | | FAM65B | 9 | 3 | 0.333333333 |
| | | CIC | 3500 | 1151 | 0.328857143 |
| | | CCM2 | 2144 | 700 | 0.326492537 |
| BI_CD4_ Memory_Primary_8pool | [‘KLF12’, | CD28 | 9013 | 8740 | 0.969710418 |
| | ‘NR4A2’, | ISG20 | 13861 | 13066 | 0.942644831 |
| | ‘STAT5B’, | IL7R | 2780 | 2436 | 0.876258993 |
| | ‘IRF1’, | CCR7 | 2514 | 2064 | 0.821002387 |
| | ‘ARID5B’]229 | TCF7 | 343 | 258 | 0.752186589 |
| | | CD6 | 407 | 300 | 0.737100737 |
| | | ZC3HAV1 | 2531 | 1685 | 0.665744765 |
| | | CD53 | 152 | 101 | 0.664473684 |
| | | ICAM2 | 316 | 176 | 0.556962025 |
| | | CD2 | 16582 | 8576 | 0.517187312 |
| | | PTPRC | 17928 | 9197 | 0.51299643 |
| | | IL10RA | 166 | 85 | 0.512048193 |
| | | DOCK8 | 90 | 45 | 0.5 |
| | | C13orf15 | 2 | 1 | 0.5 |
| | | ITGA4 | 2169 | 1082 | 0.498847395 |
| | | CLEC2D | 59 | 29 | 0.491525424 |
| | | IL16 | 733 | 348 | 0.474761255 |
| | | BCL6 | 1505 | 709 | 0.471096346 |
| | | STK17B | 42 | 18 | 0.428571429 |
| | | LAPTM5 | 31 | 12 | 0.387096774 |
| | | ITGB2 | 22607 | 8300 | 0.36714292 |
| | | AKNA | 11 | 4 | 0.363636364 |
| | | CD97 | 152 | 52 | 0.342105263 |
| | | SLAMF1 | 1911 | 639 | 0.334379906 |
| | | TNFAIP8 | 57 | 19 | 0.333333333 |
| | | CXCR4 | 9055 | 3001 | 0.331419105 |
| | | IKZF1 | 1278 | 416 | 0.325508607 |
| | | TRAF1 | 578 | 170 | 0.294117647 |
| | | FYB | 482 | 141 | 0.29253112 |
| | | KLF13 | 50 | 14 | 0.28 |
| | | STAT5B | 4280 | 1143 | 0.267056075 |
| | | KLF2 | 351 | 87 | 0.247863248 |
| | | STIM2 | 131 | 31 | 0.236641221 |
| | | ITGB1 | 5414 | 1261 | 0.232914666 |
| | | MBP | 9274 | 2151 | 0.231938754 |
| | | IER2 | 31 | 7 | 0.225806452 |
| | | ITPKB | 54 | 12 | 0.222222222 |
| | | HIVEP2 | 100 | 22 | 0.22 |
| | | LTB | 2054 | 451 | 0.219571568 |
| | | EVI2B | 19 | 4 | 0.210526316 |
| | | TRAF3IP3 | 5 | 1 | 0.2 |
| | | RUNX3 | 770 | 153 | 0.198701299 |
| | | CMAH | 41 | 8 | 0.195121951 |
| | | SELPLG | 4201 | 776 | 0.184717924 |
| | | BIRC3 | 1009 | 182 | 0.180376611 |
| | | ETS1 | 1684 | 303 | 0.179928741 |
| | | ATXN7 | 5383 | 954 | 0.177224596 |
| | | WFPF1 | 260 | 46 | 0.176923077 |
| | | SH2B3 | 291 | 50 | 0.171821306 |
| | | CSK | 2914 | 493 | 0.169183253 |
| BI_CD4_Naive_Primary_7pool | [‘STAT5B’, | PHF15 | 1 | 1 | 1 |
| | ‘NR4A2’, | GIMAP7 | 3 | 3 | 1 |
| | ‘BACH2’, | CD28 | 9013 | 8740 | 0.969710418 |
| | ‘BCL6’, | ISG20 | 13861 | 13066 | 0.942644831 |
| | ‘TGIF1’, | CD247 | 429 | 386 | 0.8997669 |
| | ‘LEF1’]230 | IL7R | 2780 | 2436 | 0.876258993 |
| | | CCR7 | 2514 | 2064 | 0.821002387 |
| | | TCF7 | 343 | 258 | 0.752186589 |
| | | CD6 | 407 | 300 | 0.737100737 |
| | | ARL4C | 3420 | 2399 | 0.701461988 |
| | | PRKCQ | 404 | 257 | 0.636138614 |
| | | ICAM2 | 316 | 176 | 0.556962025 |
| | | CD2 | 16582 | 8576 | 0.517187312 |
| | | PTPRC | 17928 | 9197 | 0.51299643 |
| | | C13orf15 | 2 | 1 | 0.5 |
| | | CLEC2D | 59 | 29 | 0.491525424 |
| | | IL16 | 733 | 348 | 0.474761255 |
| | | BCL6 | 1505 | 709 | 0.471096346 |
| | | BACH2 | 107 | 49 | 0.457943925 |
| | | GPR132 | 672 | 297 | 0.441964286 |
| | | STK17B | 42 | 18 | 0.428571429 |
| | | LAPTM5 | 31 | 12 | 0.387096774 |
| | | SELL | 10547 | 3994 | 0.378685882 |
| | | CMTM7 | 8 | 3 | 0.375 |
| | | SATB1 | 227 | 83 | 0.365638767 |
| | | AKNA | 11 | 4 | 0.363636364 |
| | | CD97 | 152 | 52 | 0.342105263 |
| | | CD40LG | 90425 | 30710 | 0.339618468 |
| | | TNFAIP8 | 57 | 19 | 0.333333333 |
| | | CXCR4 | 9055 | 3001 | 0.331419105 |
| | | IKZF1 | 1278 | 416 | 0.325508607 |
| | | NDFIP1 | 39 | 12 | 0.307692308 |
| | | LEP1 | 1327 | 408 | 0.307460437 |
| | | IL6R | 11078 | 3373 | 0.304477342 |
| | | FMNL1 | 43 | 13 | 0.302325581 |
| | | TRAF1 | 578 | 170 | 0.294117647 |
| | | FYB | 482 | 141 | 0.29253112 |
| | | GIMAP2 | 21 | 6 | 0.285714286 |
| | | KLF13 | 50 | 14 | 0.28 |
| | | STAT5B | 4280 | 1143 | 0.267056075 |
| | | KLF2 | 351 | 87 | 0.247863248 |
| | | HDAC7 | 162 | 40 | 0.24691358 |
| | | PLCG1 | 577 | 141 | 0.244367418 |
| | | B2M | 671 | 155 | 0.23099851 |
| | | IER2 | 31 | 7 | 0.225806452 |
| | | ITPKB | 54 | 12 | 0.222222222 |
| | | HIVEP2 | 100 | 22 | 0.22 |
| | | EVI2B | 19 | 4 | 0.210526316 |
| | | TRAF3IP3 | 5 | 1 | 0.2 |
| | | SELPLG | 4201 | 776 | 0.184717924 |
| BI_CD4p_CD225int_CD127p_Tmem | [‘IRF1’, | CD28 | 9013 | 8740 | 0.969710418 |
| | ‘SMAD3’, | ISG20 | 13861 | 13066 | 0.942644831 |
| | ‘STAT5B’, | TNFRSF18 | 589 | 550 | 0.933786078 |
| | ‘TGIF1’, | CD247 | 429 | 386 | 0.8997669 |
| | ‘KLF12’, | IL7R | 2780 | 2436 | 0.876258993 |
| | ‘STAT4’, | CCR7 | 2514 | 2064 | 0.821002387 |
| | ‘CREB1’]243 | NFATC2 | 496 | 406 | 0.818548387 |
| | | LCP2 | 495 | 399 | 0.806060606 |
| | | NLRC5 | 44 | 34 | 0.772727273 |
| | | GPR183 | 38 | 29 | 0.763157895 |
| | | TCF7 | 343 | 258 | 0.752186589 |
| | | CD6 | 407 | 300 | 0.737100737 |
| | | ARL4C | 3420 | 2399 | 0.701461988 |
| | | CD53 | 152 | 101 | 0.664473684 |
| | | STAT4 | 1031 | 656 | 0.636275461 |
| | | CD3D | 332 | 199 | 0.59939759 |
| | | CD2 | 16582 | 8576 | 0.517187312 |
| | | PTPRC | 17928 | 9197 | 0.51299643 |
| | | TAP1 | 1353 | 670 | 0.495195861 |
| | | CLEC2D | 59 | 29 | 0.491525424 |
| | | IL16 | 733 | 348 | 0.474761255 |
| | | GPR65 | 48 | 22 | 0.458333333 |
| | | GPR132 | 672 | 297 | 0.441964286 |
| | | STK17B | 42 | 18 | 0.428571429 |
| | | LAPTM5 | 31 | 12 | 0.387096774 |
| | | TNFAIP3 | 1645 | 612 | 0.372036474 |
| | | AKNA | 11 | 4 | 0.363636364 |
| | | CD40LG | 90425 | 30710 | 0.339618468 |
| | | SLAMF1 | 1911 | 639 | 0.334379906 |
| | | TNFAIP8 | 57 | 19 | 0.333333333 |
| | | IKZF1 | 1278 | 416 | 0.325508607 |
| | | FMNL1 | 43 | 13 | 0.302325581 |
| | | TRAF1 | 578 | 170 | 0.294117647 |
| | | FYB | 482 | 141 | 0.29253112 |
| | | KLF13 | 50 | 14 | 0.28 |
| | | STAT5B | 4280 | 1143 | 0.267056075 |
| | | NFKBIA | 272 | 70 | 0.257352941 |
| | | SOCS3 | 2033 | 505 | 0.248401377 |
| | | KLF2 | 351 | 87 | 0.247863248 |
| | | HDAC7 | 162 | 40 | 0.24691358 |
| | | PLCG1 | 577 | 141 | 0.244367418 |
| | | RCAN3 | 21 | 5 | 0.238095238 |
| | | ITGB1 | 5414 | 1261 | 0.232914666 |
| | | MBP | 9274 | 2151 | 0.231938754 |
| | | B2M | 671 | 155 | 0.23099851 |
| | | RASSF5 | 147 | 33 | 0.224489796 |
| | | SYTL3 | 18 | 4 | 0.222222222 |
| | | ITPKB | 54 | 12 | 0.222222222 |
| | | HIVEP2 | 100 | 22 | 0.22 |
| | | TNFRSF1B | 7820 | 1691 | 0.216240409 |
| BI_CD4p_CD25-_CD45RAp_Naive | [‘STAT5B’, | PHF15 | 1 | 1 | 1 |
| | ‘SREBF1’, | CD28 | 9013 | 8740 | 0.969710418 |
| | ‘IKZF1’, | ISG20 | 13861 | 13066 | 0.942644831 |
| | ‘NR4A2’, | CD247 | 429 | 386 | 0.8997669 |
| | ‘BACH2’]402 | IL7R | 2780 | 2436 | 0.876258993 |
| | | LCK | 3367 | 2863 | 0.85031185 |
| | | CCR7 | 2514 | 2064 | 0.821002387 |
| | | LCP2 | 495 | 399 | 0.806060606 |
| | | NLRC5 | 44 | 34 | 0.772727273 |
| | | TCF7 | 343 | 258 | 0.752186589 |
| | | CD6 | 407 | 300 | 0.737100737 |
| | | IL4R | 6442 | 4568 | 0.709096554 |
| | | ARL4C | 3420 | 2399 | 0.701461988 |
| | | MYL12B | 855 | 598 | 0.699415205 |
| | | ZBTB7B | 82 | 57 | 0.695121951 |
| | | GIMAP5 | 74 | 51 | 0.689189189 |
| | | ZC3HAV1 | 2531 | 1685 | 0.665744765 |
| | | CD53 | 152 | 101 | 0.664473684 |
| | | MYADM | 11 | 7 | 0.636363636 |
| | | ZNF395 | 6714 | 4097 | 0.610217456 |
| | | ICAM2 | 316 | 176 | 0.556962025 |
| | | SIRPG | 17 | 9 | 0.529411765 |
| | | CD2 | 16582 | 8576 | 0.517187312 |
| | | TRIM69 | 948 | 489 | 0.515822785 |
| | | PTPRC | 17928 | 9197 | 0.51299643 |
| | | KIAA0922 | 2 | 1 | 0.5 |
| | | C13orf15 | 2 | 1 | 0.5 |
| | | VAV1 | 1267 | 633 | 0.499605367 |
| | | CLEC2D | 59 | 29 | 0.491525424 |
| | | IL16 | 733 | 348 | 0.474761255 |
| | | BACH2 | 107 | 49 | 0.457943925 |
| | | UNC13D | 165 | 75 | 0.454545455 |
| | | GPR132 | 672 | 297 | 0.441964286 |
| | | STK17B | 42 | 18 | 0.428571429 |
| | | ZBTB1 | 5 | 2 | 0.4 |
| | | HIST1H2BD | 5 | 2 | 0.4 |
| | | IL18BP | 23 | 9 | 0.391304348 |
| | | LAPTM5 | 31 | 12 | 0.387096774 |
| | | PSMB8 | 690 | 264 | 0.382608696 |
| | | CMTM7 | 8 | 3 | 0.375 |
| | | TNFAIP3 | 1645 | 612 | 0.372036474 |
| | | SATB1 | 227 | 83 | 0.365638767 |
| | | AKNA | 11 | 4 | 0.363636364 |
| | | ELF1 | 109 | 39 | 0.357798165 |
| | | CD97 | 152 | 52 | 0.342105263 |
| | | CD40LG | 90425 | 30710 | 0.339618468 |
| | | SLAMF1 | 1911 | 639 | 0.334379906 |
| | | TNFAIP8 | 57 | 19 | 0.333333333 |
| | | FASN | 26569 | 8843 | 0.332831495 |
| | | CXCR4 | 9055 | 3001 | 0.331419105 |
| BI_CD4p_CD25-_CD45ROp_Memory | [‘RFX1’, | PHF15 | 1 | 1 | 1 |
| | ‘SMAD3’, | CD28 | 9013 | 8740 | 0.969710418 |
| | ‘STAT5B’, | ISG20 | 13861 | 13066 | 0.942644831 |
| | ‘IKZF1’, | CD3G | 327 | 295 | 0.902140673 |
| | ‘TGIF1’, | CD247 | 429 | 386 | 0.8997669 |
| | ‘NR4A2’, | IL7R | 2780 | 2436 | 0.876258993 |
| | ‘REL’]393 | LCK | 3367 | 2863 | 0.85031185 |
| | | CXCR5 | 600 | 495 | 0.825 |
| | | CCR7 | 2514 | 2064 | 0.821002387 |
| | | NFATC2 | 496 | 406 | 0.818548387 |
| | | LCP2 | 495 | 399 | 0.806060606 |
| | | NLRC5 | 44 | 34 | 0.772727273 |
| | | GPR183 | 38 | 29 | 0.763157895 |
| | | TCF7 | 343 | 258 | 0.752186589 |
| | | ARL4C | 3420 | 2399 | 0.701461988 |
| | | ZBTB7B | 82 | 57 | 0.695121951 |
| | | ZC3HAV1 | 2531 | 1685 | 0.665744765 |
| | | PRKCQ | 404 | 257 | 0.636138614 |
| | | BATF | 95 | 60 | 0.631578947 |
| | | CD2 | 16582 | 8576 | 0.517187312 |
| | | PTPRC | 17928 | 9197 | 0.51299643 |
| | | IL10RA | 166 | 85 | 0.512048193 |
| | | KIAA0922 | 2 | 1 | 0.5 |
| | | DOCK8 | 90 | 45 | 0.5 |
| | | CLEC2D | 59 | 29 | 0.491525424 |
| | | IL16 | 733 | 348 | 0.474761255 |
| | | GPR132 | 672 | 297 | 0.441964286 |
| | | STK17B | 42 | 18 | 0.428571429 |
| | | ZBTB1 | 5 | 2 | 0.4 |
| | | LAPTM5 | 31 | 12 | 0.387096774 |
| | | IRAK2 | 993 | 383 | 0.385699899 |
| | | PSMB8 | 690 | 264 | 0.382608696 |
| | | CMTM7 | 8 | 3 | 0.375 |
| | | TNFAIP3 | 1645 | 612 | 0.372036474 |
| | | TAGAP | 27 | 10 | 0.37037037 |
| | | ITGB2 | 22607 | 8300 | 0.36714292 |
| | | AKNA | 11 | 4 | 0.363636364 |
| | | ELF1 | 109 | 39 | 0.357798165 |
| | | HLA-C | 2739 | 960 | 0.350492881 |
| | | CD97 | 152 | 52 | 0.342105263 |
| | | CD40LG | 90425 | 30710 | 0.339618468 |
| | | SLAMF1 | 1911 | 639 | 0.334379906 |
| | | TNFAIP8 | 57 | 19 | 0.333333333 |
| | | CXCR4 | 9055 | 3001 | 0.331419105 |
| | | ORAI2 | 52 | 17 | 0.326923077 |
| | | IKZF1 | 1278 | 416 | 0.325508607 |
| | | STAT1 | 5790 | 1873 | 0.323488774 |
| | | HLA-B | 11036 | 3546 | 0.32131207 |
| | | GPBP1 | 51 | 16 | 0.31372549 |
| | | REL | 3847 | 1181 | 0.306992462 |
| BI_CD8_Memory_7pool | [‘IRF1’, | ISG20 | 13861 | 13066 | 0.942644831 |
| | ‘SMAD3’, | TIGIT | 26 | 24 | 0.923076923 |
| | ‘STAT5B’, | IL7R | 2780 | 2436 | 0.876258993 |
| | ‘SREBF1’, | CCR7 | 2514 | 2064 | 0.821002387 |
| | ‘TGIF1’, | NFATC2 | 496 | 406 | 0.818548387 |
| | ‘REL’, | LCP2 | 495 | 399 | 0.806060606 |
| | ‘RREB1’, | CD84 | 71 | 57 | 0.802816901 |
| | ‘NR4A2’]437 | KLRK1 | 1692 | 1294 | 0.764775414 |
| | | GPR183 | 38 | 29 | 0.763157895 |
| | | TCF7 | 343 | 258 | 0.752186589 |
| | | NFATC3 | 215 | 153 | 0.711627907 |
| | | ARL4C | 3420 | 2399 | 0.701461988 |
| | | FCGR3B | 6753 | 4537 | 0.671849548 |
| | | FCGR3A | 6819 | 4551 | 0.667399912 |
| | | ZC3HAV1 | 2531 | 1685 | 0.665744765 |
| | | CD53 | 132 | 101 | 0.664473684 |
| | | MYADM | 11 | 7 | 0.636363636 |
| | | CD8A | 118848 | 71224 | 0.599286484 |
| | | CD2 | 16582 | 8576 | 0.517187312 |
| | | PTPRC | 17928 | 9197 | 0.51299643 |
| | | IL10RA | 166 | 85 | 0.512048193 |
| | | DOCK8 | 90 | 45 | 0.5 |
| | | CLEC2D | 59 | 29 | 0.491525424 |
| | | IL16 | 733 | 348 | 0.474761255 |
| | | BCL6 | 1505 | 709 | 0.471096346 |
| | | GPR65 | 48 | 22 | 0.458333333 |
| | | STK17B | 42 | 18 | 0.428571429 |
| | | TARP | 545 | 215 | 0.394495413 |
| | | LAPTM5 | 31 | 12 | 0.387096774 |
| | | FHL3 | 67 | 25 | 0.373134328 |
| | | TNFAIP3 | 1645 | 612 | 0.372036474 |
| | | AKNA | 11 | 4 | 0.363636364 |
| | | SIGLEC6 | 17 | 6 | 0.352941176 |
| | | CD97 | 152 | 52 | 0.342105263 |
| | | TNFAIP8 | 57 | 19 | 0.333333333 |
| | | CXCR4 | 9055 | 3001 | 0.331419105 |
| | | IKZF1 | 1278 | 416 | 0.325508607 |
| | | HLA-B | 11036 | 3546 | 0.32131207 |
| | | GPBP1 | 51 | 16 | 0.31372549 |
| | | IER5 | 13 | 4 | 0.307692308 |
| | | REL | 3847 | 1181 | 0.306992462 |
| | | PTPN7 | 88 | 27 | 0.306818182 |
| | | FMNL1 | 43 | 13 | 0.302325581 |
| | | ARHGEF2 | 7034 | 2074 | 0.294853568 |
| | | TRAF1 | 578 | 170 | 0.294117647 |
| | | FYB | 482 | 141 | 0.29253112 |
| | | KLF13 | 50 | 14 | 0.28 |
| | | STAT5B | 4280 | 1143 | 0.267056075 |
| | | MIR223 | 315 | 83 | 0.263492063 |
| | | NFKB2 | 1866 | 478 | 0.256162915 |
| BI_CD8_Naive_7pool | [‘IRF1’, | PHF15 | 1 | 1 | 1 |
| | ‘NR4A2’, | KLRAP1 | 13 | 13 | 1 |
| | ‘LEF1’, | GIMAP7 | 3 | 3 | 1 |
| | ‘TGIF1’, | ISG20 | 13861 | 13066 | 0.942644831 |
| | ‘BCL6’, | CD247 | 429 | 386 | 0.8997669 |
| | ‘BACH2’]245 | IL7R | 2780 | 2436 | 0.876258993 |
| | | CCR7 | 2514 | 2064 | 0.821002387 |
| | | LCP2 | 495 | 399 | 0.806060606 |
| | | NLRC5 | 44 | 34 | 0.772727273 |
| | | KLRK1 | 1692 | 1294 | 0.764775414 |
| | | TCF7 | 343 | 258 | 0.752186589 |
| | | CD6 | 407 | 300 | 0.737100737 |
| | | ARL4C | 3420 | 2399 | 0.701461988 |
| | | CD53 | 152 | 101 | 0.664473684 |
| | | CD8A | 118848 | 71224 | 0.599286484 |
| | | ICAM2 | 316 | 176 | 0.556962025 |
| | | CD2 | 16582 | 8576 | 0.517187312 |
| | | PTPRC | 17928 | 9197 | 0.51299643 |
| | | DOCK8 | 90 | 45 | 0.5 |
| | | C13orf15 | 2 | 1 | 0.5 |
| | | CLEC2D | 59 | 29 | 0.491525424 |
| | | IL16 | 733 | 348 | 0.474761255 |
| | | BCL6 | 1505 | 709 | 0.471096346 |
| | | BACH2 | 107 | 49 | 0.457943925 |
| | | GPR132 | 672 | 297 | 0.441964286 |
| | | MIR142 | 69 | 30 | 0.434782609 |
| | | STK17B | 42 | 18 | 0.428571429 |
| | | HIST1H2BD | 5 | 2 | 0.4 |
| | | LAPTM5 | 31 | 12 | 0.387096774 |
| | | TNFAIP3 | 1645 | 612 | 0.372036474 |
| | | SATB1 | 227 | 83 | 0.365638767 |
| | | AKNA | 11 | 4 | 0.363636364 |
| | | CD97 | 152 | 52 | 0.342105263 |
| | | SDCCAG1 | 3 | 1 | 0.333333333 |
| | | CXCR4 | 9055 | 3001 | 0.331419105 |
| | | IKZF1 | 1278 | 416 | 0.325508607 |
| | | NDFIP1 | 39 | 12 | 0.307692308 |
| | | LEF1 | 1327 | 408 | 0.307460437 |
| | | FMNL1 | 43 | 13 | 0.302325581 |
| | | TRAF1 | 578 | 170 | 0.294117647 |
| | | FYB | 482 | 141 | 0.29253112 |
| | | GIMAP2 | 21 | 6 | 0.285714286 |
| | | KLF13 | 50 | 14 | 0.28 |
| | | MIR1205 | 4 | 1 | 0.25 |
| | | IRF2BP2 | 12 | 3 | 0.25 |
| | | KLF2 | 351 | 87 | 0.247863248 |
| | | PLCG1 | 577 | 141 | 0.244367418 |
| | | STIM2 | 131 | 31 | 0.236641221 |
| | | B2M | 671 | 155 | 0.23099851 |
| | | IER2 | 31 | 7 | 0.225806452 |
| BI_Duodenum_Smooth_Muscle | [‘IRF2’, | DCAF5 | 3 | 3 | 1 |
| | ‘NR4A1’, | C15orf52 | 1 | 1 | 1 |
| | ‘ZBTB16’, | ACTA2 | 728 | 486 | 0.667582418 |
| | ‘TCF7L2’, | CDX1 | 240 | 138 | 0.575 |
| | ‘HIF1A’, | MEF2D | 168 | 89 | 0.529761905 |
| | ‘SMAD3’, | CDX2 | 1304 | 619 | 0.474693252 |
| | ‘HOXA4’, | MYLK | 4842 | 2150 | 0.444031392 |
| | ‘ELF3’, | MRVI1 | 45 | 15 | 0.333333333 |
| | ‘RREB1’, | PPP1R12B | 20 | 6 | 0.3 |
| | ‘NR4A2’, | MYH11 | 579 | 172 | 0.297063903 |
| | ‘ARID5B’, | KLF5 | 348 | 103 | 0.295977011 |
| | ‘TGIF1’]514 | GJC1 | 386 | 113 | 0.292746114 |
| | | SLC40A1 | 323 | 93 | 0.287925697 |
| | | PIGR | 350 | 99 | 0.282857143 |
| | | NKX2-3 | 64 | 17 | 0.265625 |
| | | GNAI2 | 2970 | 746 | 0.251178451 |
| | | KIAA0247 | 4 | 1 | 0.25 |
| | | C9orf5 | 4 | 1 | 0.25 |
| | | CUBN | 101 | 24 | 0.237623762 |
| | | GATA6 | 527 | 110 | 0.208728653 |
| | | SLC9A1 | 1428 | 264 | 0.18487395 |
| | | SYNPO2 | 33 | 6 | 0.181818182 |
| | | SLC7A8 | 223 | 37 | 0.165919283 |
| | | CACNB2 | 80 | 13 | 0.1625 |
| | | ESYT2 | 13 | 2 | 0.153846154 |
| | | TINAGL1 | 744 | 112 | 0.150537634 |
| | | JPH2 | 173 | 26 | 0.150289017 |
| | | CELF2 | 95 | 14 | 0.147368421 |
| | | PTGIS | 694 | 102 | 0.146974063 |
| | | SMAD7 | 1310 | 192 | 0.146564885 |
| | | CORO1C | 7 | 1 | 0.142857143 |
| | | AFAP1-AS1 | 7 | 1 | 0.142857143 |
| | | KLF6 | 2304 | 310 | 0.134548611 |
| | | SMAD3 | 3407 | 449 | 0.131787496 |
| | | ATP1B1 | 92 | 12 | 0.130434783 |
| | | IQGAP1 | 1745 | 227 | 0.13008596 |
| | | PTGER4 | 1788 | 224 | 0.125279642 |
| | | ATP2B4 | 254 | 31 | 0.122047244 |
| | | AFAP1 | 115 | 14 | 0.12173913 |
| | | GRK5 | 309 | 37 | 0.1197411 |
| | | TCF7L2 | 1739 | 204 | 0.117308798 |
| | | AKAP1 | 520 | 61 | 0.117307692 |
| | | AHNAK | 95 | 11 | 0.115789474 |
| | | CAV1 | 5940 | 677 | 0.113973064 |
| | | ADCY5 | 213 | 23 | 0.107981221 |
| | | DHRS3 | 65 | 7 | 0.107692308 |
| | | S100A11 | 177 | 19 | 0.107344633 |
| | | BMPR1A | 853 | 90 | 0.105509965 |
| | | HOXA4 | 152 | 16 | 0.105263158 |
| | | TGFBR2 | 519 | 54 | 0.104046243 |
| BI_Skeletal_Muscle | [‘ARID5B’, | ZCCHC24 | 1 | 1 | 1 |
| | ‘ZBTB16’, | SMTNL2 | 1 | 1 | 1 |
| | ‘NFE2L1’, | FBXO32 | 488 | 478 | 0.979508197 |
| | ‘NR4A1’, | OBSCN | 46 | 44 | 0.956521739 |
| | ‘RREB1’, | MYF6 | 437 | 413 | 0.945080092 |
| | ‘SREBF1’, | MYL1 | 98 | 90 | 0.918367347 |
| | ‘ZNP423’, | MYH2 | 100 | 91 | 0.91 |
| | ‘TGIF1’, | LMOD2 | 6 | 5 | 0.833333333 |
| | ‘SMAD3’]515 | MYOT | 101 | 83 | 0.821782178 |
| | | XIRP2 | 22 | 18 | 0.818181818 |
| | | CMYA5 | 19 | 15 | 0.789473684 |
| | | MYOD1 | 3844 | 2978 | 0.77471384 |
| | | NRAP | 49 | 37 | 0.755102041 |
| | | MYPN | 16 | 12 | 0.75 |
| | | MEF2D | 168 | 126 | 0.75 |
| | | TBC1D4 | 303 | 225 | 0.742574237 |
| | | MYOF | 37 | 27 | 0.72972973 |
| | | MYBPC1 | 17 | 12 | 0.705882353 |
| | | TNNT3 | 47 | 33 | 0.70212766 |
| | | MEF2C | 622 | 436 | 0.70096463 |
| | | RBM24 | 10 | 7 | 0.7 |
| | | TRIM54 | 291 | 202 | 0.694158076 |
| | | VGLL2 | 13 | 9 | 0.692307692 |
| | | ITGA7 | 102 | 69 | 0.676470588 |
| | | CAPN3 | 481 | 324 | 0.673596674 |
| | | ACTN2 | 63 | 41 | 0.650793651 |
| | | SORBS3 | 57 | 36 | 0.631578947 |
| | | TXLNB | 8 | 5 | 0.625 |
| | | KLHL31 | 8 | 5 | 0.625 |
| | | CACNG1 | 13 | 8 | 0.615384615 |
| | | FOXK1 | 36 | 21 | 0.583333333 |
| | | PFKM | 511 | 292 | 0.571428571 |
| | | DUSP27 | 7 | 4 | 0.571428571 |
| | | SCN4A | 839 | 473 | 0.563766389 |
| | | CACNA1S | 877 | 451 | 0.514253136 |
| | | TMEM182 | 2 | 1 | 0.5 |
| | | RBM20 | 16 | 8 | 0.5 |
| | | KBTBD10 | 8 | 4 | 0.5 |
| | | SYNPO2 | 33 | 14 | 0.424242424 |
| | | TPM1 | 243 | 100 | 0.411522634 |
| | | PLB1 | 1114 | 419 | 0.376122083 |
| | | FABP3 | 744 | 269 | 0.36155914 |
| | | PPARGC1B | 213 | 75 | 0.352112676 |
| | | ADSSL1 | 3 | 1 | 0.333333333 |
| | | ABLIM2 | 3 | 1 | 0.333333333 |
| | | CNBP | 6556 | 2124 | 0.323978035 |
| | | CAPZB | 291 | 94 | 0.323024055 |
| | | PLN | 1996 | 632 | 0.316633267 |
| | | ZFAND5 | 10 | 3 | 0.3 |
| | | BTBD1 | 10 | 3 | 0.3 |
| BI_Stomach_Smooth_Muscle | [‘NR4A1’, | C15orf52 | 1 | 1 | 1 |
| | ‘GTF2IRD1’, | SMTN | 96 | 75 | 0.78125 |
| | ‘TGIF1’, | MYOCD | 68 | 53 | 0.779411765 |
| | ‘RREB1’, | ACTA2 | 728 | 488 | 0.67032967 |
| | ‘NR4A2’, | GNAI2 | 2970 | 1716 | 0.577777778 |
| | ‘SREBF1’]543 | MEF2D | 168 | 89 | 0.529761905 |
| | | KIAA1274 | 2 | 1 | 0.5 |
| | | MYLK | 4842 | 2018 | 0.41676993 |
| | | TAGLN | 828 | 310 | 0.374396135 |
| | | MYL9 | 336 | 118 | 0.351190476 |
| | | NT5DC3 | 3 | 1 | 0.333333333 |
| | | AHNAK2 | 3 | 1 | 0.333333333 |
| | | MRVI1 | 45 | 14 | 0.311111111 |
| | | PPP1R12B | 20 | 6 | 0.3 |
| | | MYH11 | 579 | 170 | 0.293609672 |
| | | GJC1 | 386 | 111 | 0.287564767 |
| | | BARX1 | 58 | 13 | 0.224137931 |
| | | DNAJB5 | 5 | 1 | 0.2 |
| | | MIR143 | 124 | 24 | 0.193548387 |
| | | TRAK1 | 21 | 4 | 0.19047619 |
| | | JAG1 | 7483 | 1385 | 0.185086195 |
| | | WNT9A | 76 | 14 | 0.184210526 |
| | | SYNPO2 | 33 | 6 | 0.181818182 |
| | | TEAD3 | 40 | 7 | 0.175 |
| | | PDGFC | 155 | 26 | 0.167741935 |
| | | SLC45A1 | 6 | 1 | 0.166666667 |
| | | NKD1 | 43 | 7 | 0.162790698 |
| | | CACNB2 | 80 | 13 | 0.1625 |
| | | MIR145 | 481 | 77 | 0.16008316 |
| | | HDAC7 | 162 | 24 | 0.148148148 |
| | | AFAP1 | 115 | 17 | 0.147826087 |
| | | CACNA1H | 240 | 35 | 0.145833333 |
| | | JPH2 | 173 | 25 | 0.144508671 |
| | | RAMP1 | 335 | 48 | 0.143283582 |
| | | RGS3 | 112 | 16 | 0.142857143 |
| | | ISL1 | 825 | 117 | 0.141818182 |
| | | TACC1 | 43 | 6 | 0.139534884 |
| | | CAMK2G | 793 | 107 | 0.134930643 |
| | | SMAD7 | 1310 | 176 | 0.134351145 |
| | | RGMA | 626 | 83 | 0.132587859 |
| | | ADCY5 | 213 | 27 | 0.126760563 |
| | | WISP1 | 158 | 20 | 0.126582278 |
| | | TP53I11 | 16 | 2 | 0.125 |
| | | KCNH2 | 3015 | 370 | 0.122719735 |
| | | TPM2 | 640 | 77 | 0.1203125 |
| | | GRK5 | 309 | 37 | 0.1197411 |
| | | AKAP1 | 520 | 62 | 0.119230769 |
| | | AHNAK | 95 | 11 | 0.115789474 |
| | | TINAGL1 | 744 | 85 | 0.114247312 |
| | | LIMS2 | 27 | 3 | 0.111111111 |
| CD14 | [‘IRF2’, | C19orf61 | 1 | 1 | 1 |
| | ‘BACH1’, | LAIR1 | 96 | 71 | 0.739583333 |
| | ‘SMAD3’, | LRRC8D | 3 | 2 | 0.666666667 |
| | ‘KLF4’, | CCR2 | 2787 | 1836 | 0.658772874 |
| | ‘IKZF1’, | CCR1 | 1192 | 744 | 0.624161074 |
| | ‘MAX’, | IRAK3 | 126 | 72 | 0.571428571 |
| | ‘FLI1’]859 | ITGAX | 4499 | 2436 | 0.541453656 |
| | | PDE4DIP | 35 | 18 | 0.514285714 |
| | | CAPG | 18504 | 9413 | 0.508700821 |
| | | SIGLEC9 | 61 | 31 | 0.508196721 |
| | | LRRC33 | 2 | 1 | 0.5 |
| | | TREM1 | 393 | 193 | 0.491094148 |
| | | CX3CR1 | 1055 | 500 | 0.473933649 |
| | | TLR2 | 6189 | 2887 | 0.466472774 |
| | | AOAH | 32 | 14 | 0.4375 |
| | | SIGLEC5 | 78 | 34 | 0.435897436 |
| | | CD86 | 7694 | 3341 | 0.434234468 |
| | | CD97 | 152 | 65 | 0.427631579 |
| | | FCGR3B | 6753 | 2878 | 0.426180957 |
| | | FCGR3A | 6819 | 2882 | 0.422642616 |
| | | TM9SF4 | 5 | 2 | 0.4 |
| | | FCN1 | 20 | 8 | 0.4 |
| | | AIM2 | 222 | 88 | 0.396396396 |
| | | IRF8 | 461 | 179 | 0.388286334 |
| | | C3AR1 | 220 | 81 | 0.368181818 |
| | | CD84 | 71 | 25 | 0.352112676 |
| | | SPI1 | 2118 | 735 | 0.347025496 |
| | | SCARB1 | 2019 | 684 | 0.338781575 |
| | | C20orf3 | 3 | 1 | 0.333333333 |
| | | ALOX5 | 3395 | 1111 | 0.32724595 |
| | | MNDA | 77 | 24 | 0.311688312 |
| | | IL16 | 733 | 228 | 0.311050477 |
| | | PILRA | 27 | 8 | 0.296296296 |
| | | CD58 | 1619 | 468 | 0.289067326 |
| | | LCP2 | 495 | 141 | 0.284848485 |
| | | IL10RA | 166 | 47 | 0.28313253 |
| | | PTAFR | 202 | 57 | 0.282178218 |
| | | STX11 | 58 | 16 | 0.275862069 |
| | | IL4R | 6442 | 1717 | 0.266532133 |
| | | MYO18A | 27 | 7 | 0.259259259 |
| | | IL6R | 11078 | 2848 | 0.257086117 |
| | | P2RX7 | 1675 | 419 | 0.250149254 |
| | | LRRFIP2 | 12 | 3 | 0.25 |
| | | KIAA0247 | 4 | 1 | 0.25 |
| | | IL1RN | 6571 | 1600 | 0.243494141 |
| | | GPR183 | 38 | 9 | 0.236842105 |
| | | TNFRSF10B | 58857 | 13879 | 0.235808825 |
| | | IL17RA | 282 | 66 | 0.234042553 |
| | | CD180 | 121 | 28 | 0.231404959 |
| | | CYTH4 | 13 | 3 | 0.230769231 |
| CD19_primary | [‘NR4A2’, | LRRC33 | 2 | 2 | 1 |
| | ‘FLI1’, | IGLL5 | 1 | 1 | 1 |
| | ‘SMAD3’, | CLEC17A | 1 | 1 | 1 |
| | ‘SPIB’, | C14orf43 | 1 | 1 | 1 |
| | ‘CTCF’, | CD72 | 223 | 216 | 0.968609865 |
| | ‘IKZF1’, | BTLA | 195 | 179 | 0.917948718 |
| | ‘IRF2’, | ISG20 | 13861 | 12559 | 0.906067383 |
| | ‘RFX1’, | CD22 | 1698 | 1454 | 0.856301531 |
| | ‘TGIF1’]520 | ICOSLG | 353 | 299 | 0.847025496 |
| | | FCER2 | 2768 | 2302 | 0.831647399 |
| | | CXCR5 | 600 | 498 | 0.83 |
| | | LY9 | 69 | 55 | 0.797101449 |
| | | CD180 | 121 | 95 | 0.785123967 |
| | | CCR7 | 2514 | 1934 | 0.769291965 |
| | | PAX5 | 1110 | 852 | 0.767567568 |
| | | CD83 | 2204 | 1653 | 0.75 |
| | | CD37 | 212 | 154 | 0.726415094 |
| | | POU2AF1 | 210 | 151 | 0.719047619 |
| | | TNFRSF13B | 1316 | 906 | 0.688449848 |
| | | CD53 | 152 | 101 | 0.664473684 |
| | | SPIB | 139 | 88 | 0.633093525 |
| | | RCSD1 | 8 | 5 | 0.625 |
| | | P2RY8 | 24 | 15 | 0.625 |
| | | BACH2 | 107 | 65 | 0.607476636 |
| | | CIITA | 771 | 462 | 0.59922179 |
| | | HLA-DMB | 343 | 200 | 0.583090379 |
| | | AIM2 | 222 | 128 | 0.576576577 |
| | | CCR6 | 1258 | 707 | 0.56200318 |
| | | RFX5 | 106 | 59 | 0.556603774 |
| | | SWAP70 | 76 | 41 | 0.539473684 |
| | | TREML2 | 17 | 9 | 0.529411765 |
| | | PTPRC | 17928 | 9128 | 0.509147702 |
| | | PILRB | 12 | 6 | 0.5 |
| | | CMTM7 | 8 | 4 | 0.5 |
| | | C12orf35 | 2 | 1 | 0.5 |
| | | IRF8 | 461 | 221 | 0.479392625 |
| | | CLEC2D | 59 | 28 | 0.474576271 |
| | | IL10RA | 166 | 77 | 0.463855422 |
| | | CD79B | 1660 | 763 | 0.459638554 |
| | | TMSB10 | 107 | 48 | 0.448598131 |
| | | IRF5 | 329 | 146 | 0.443768997 |
| | | IL16 | 733 | 320 | 0.436562074 |
| | | MIR142 | 69 | 30 | 0.434782609 |
| | | PLCG2 | 30 | 13 | 0.433333333 |
| | | VPREB1 | 365 | 158 | 0.432876712 |
| | | ENTPD1 | 779 | 337 | 0.432605905 |
| | | GPR132 | 672 | 286 | 0.425595238 |
| | | NFATC1 | 3400 | 1429 | 0.420294118 |
| | | LAPTM5 | 31 | 13 | 0.419354839 |
| | | BTG1 | 110 | 46 | 0.418181818 |
| CD20 | [‘SREBF2’, | IGLL5 | 1 | 1 | 1 |
| | ‘ARID5B’, | CLEC17A | 1 | 1 | 1 |
| | ‘ZBTB16’, | C14orf43 | 1 | 1 | 1 |
| | ‘SP3’, | ISG20 | 13861 | 12559 | 0.906067383 |
| | ‘FLI1’, | CD22 | 1698 | 1454 | 0.856301531 |
| | ‘HIF1A’, | ICOSLG | 353 | 299 | 0.847025496 |
| | ‘SMAD3’, | IL2RA | 30293 | 25331 | 0.836199782 |
| | ‘NR4A2’, | FCER2 | 2768 | 2302 | 0.831647399 |
| | ‘SPIB’, | CXCR5 | 600 | 498 | 0.83 |
| | ‘TGIF1’]458 | LY9 | 69 | 55 | 0.797101449 |
| | | CCR7 | 2514 | 1934 | 0.769291965 |
| | | IL21R | 767 | 575 | 0.749674055 |
| | | CD37 | 212 | 154 | 0.726415094 |
| | | POU2AF1 | 210 | 151 | 0.719047619 |
| | | MYL12B | 855 | 596 | 0.697076023 |
| | | TNFRSF13B | 1316 | 906 | 0.688449848 |
| | | CD53 | 152 | 101 | 0.664473684 |
| | | SPIB | 139 | 88 | 0.633093325 |
| | | RCSD1 | 8 | 5 | 0.625 |
| | | TCL1A | 295 | 183 | 0.620338983 |
| | | CIITA | 771 | 462 | 0.59922179 |
| | | AIM2 | 222 | 128 | 0.576576577 |
| | | SWAP70 | 76 | 41 | 0.539473684 |
| | | IFNAR2 | 2107 | 1098 | 0.521120076 |
| | | PTPRC | 17928 | 9128 | 0.509147702 |
| | | C12orf35 | 2 | 1 | 0.5 |
| | | ITGA4 | 2169 | 1050 | 0.484094053 |
| | | IRF8 | 461 | 221 | 0.479392625 |
| | | IL10RA | 166 | 77 | 0.463855422 |
| | | MALT1 | 1159 | 535 | 0.461604832 |
| | | IL16 | 733 | 320 | 0.436562074 |
| | | MIR142 | 69 | 30 | 0.434782609 |
| | | PLCG2 | 30 | 13 | 0.433333333 |
| | | VPREB1 | 365 | 158 | 0.432876712 |
| | | ENTPD1 | 779 | 337 | 0.432605905 |
| | | GPR132 | 672 | 286 | 0.425595238 |
| | | NFATC1 | 3400 | 1429 | 0.420294118 |
| | | LAPTM5 | 31 | 13 | 0.419354839 |
| | | BTG1 | 110 | 46 | 0.418181818 |
| | | TOR1AIP1 | 387 | 158 | 0.408268734 |
| | | ZBTB1 | 5 | 2 | 0.4 |
| | | CD79A | 45509 | 18126 | 0.398294843 |
| | | TRAF5 | 155 | 60 | 0.387096774 |
| | | SELL | 10547 | 3912 | 0.37091116 |
| | | ITGB2 | 22607 | 8153 | 0.36064051 |
| | | STK17B | 42 | 15 | 0.357142857 |
| | | LRMP | 31 | 11 | 0.35483871 |
| | | PLXNC1 | 17 | 6 | 0.352941176 |
| | | SLAMF1 | 1911 | 636 | 0.332810047 |
| | | CD97 | 152 | 49 | 0.322368421 |
| CD3 | [‘SMAD3’, | GIMAP7 | 3 | 3 | 1 |
| | ‘SREBF1’, | CLLU1 | 18 | 18 | 1 |
| | ‘TGIF1’, | CD28 | 9013 | 8740 | 0.969710418 |
| | ‘KLF12’ | ISG20 | 13861 | 13066 | 0.942644831 |
| | ‘FLI1’, | CD247 | 429 | 386 | 0.8997669 |
| | ‘NR4A2’, | TBX21 | 1698 | 1490 | 0.877502945 |
| | ‘STAT5B’]445 | IL7R | 2780 | 2436 | 0.876258993 |
| | | LCK | 3367 | 2863 | 0.85031185 |
| | | IL2RB | 1371 | 1155 | 0.842450766 |
| | | CXCR5 | 600 | 495 | 0.825 |
| | | CCR7 | 2514 | 2064 | 0.821002387 |
| | | LCP2 | 495 | 399 | 0.806060606 |
| | | CD84 | 71 | 57 | 0.802816901 |
| | | SKAP1 | 55 | 44 | 0.8 |
| | | NLRC5 | 44 | 34 | 0.772727273 |
| | | GPR183 | 38 | 29 | 0.763157895 |
| | | TCF7 | 343 | 258 | 0.752186589 |
| | | CD6 | 407 | 300 | 0.737100737 |
| | | ARL4C | 3420 | 2399 | 0.701461988 |
| | | ZBTB7B | 82 | 57 | 0.695121951 |
| | | FCGR3B | 6753 | 4537 | 0.671849548 |
| | | FCGR3A | 6819 | 4551 | 0.667399912 |
| | | ZC3HAV1 | 2531 | 1685 | 0.665744765 |
| | | CD53 | 152 | 101 | 0.664473684 |
| | | MYADM | 11 | 7 | 0.636363636 |
| | | PRKCQ | 404 | 257 | 0.636138614 |
| | | BATF | 95 | 60 | 0.631578947 |
| | | CD3E | 398 | 242 | 0.608040201 |
| | | CD8A | 118848 | 71224 | 0.599286484 |
| | | SIRPG | 17 | 9 | 0.529411765 |
| | | CD2 | 16582 | 8576 | 0.517187312 |
| | | PTPRC | 17928 | 9197 | 0.51299643 |
| | | IL10RA | 166 | 85 | 0.512048193 |
| | | PILRB | 12 | 6 | 0.5 |
| | | KIAA0922 | 2 | 1 | 0.5 |
| | | DOCK8 | 90 | 45 | 0.5 |
| | | ITGA4 | 2169 | 1082 | 0.498847395 |
| | | IL16 | 733 | 348 | 0.474761255 |
| | | BCL6 | 1505 | 709 | 0.471096346 |
| | | GPR65 | 48 | 22 | 0.458333333 |
| | | GPR132 | 672 | 297 | 0.441964286 |
| | | STK17B | 42 | 18 | 0.428571429 |
| | | TARP | 545 | 215 | 0.394495413 |
| | | LAPTM5 | 31 | 12 | 0.387096774 |
| | | IRAK2 | 993 | 383 | 0.385699899 |
| | | PSMB8 | 690 | 264 | 0.382608696 |
| | | CIC | 3500 | 1316 | 0.376 |
| | | CMTM7 | 8 | 3 | 0.375 |
| | | TNFAIP3 | 1645 | 612 | 0.372036474 |
| | | AKNA | 11 | 4 | 0.363636364 |
| CD34_adult | [‘ELF2’, | ZNF429 | 1 | 1 | 1 |
| | ‘RREB1’, | CD34 | 26251 | 20393 | 0.776846596 |
| | ‘STAT5A’, | GFI1B | 72 | 54 | 0.75 |
| | ‘SREBF1’, | CD58 | 1619 | 1126 | 0.695491044 |
| | ‘IKZF1’]193 | HEMGN | 32 | 21 | 0.65625 |
| | | SLC25A37 | 12163 | 7342 | 0.603633972 |
| | | TBCC | 2718 | 1639 | 0.603016924 |
| | | LYL1 | 65 | 39 | 0.6 |
| | | MIR142 | 69 | 40 | 0.579710145 |
| | | TM9SF3 | 49 | 28 | 0.571428571 |
| | | RHD | 2342 | 1272 | 0.543125534 |
| | | LGALS9 | 212 | 106 | 0.5 |
| | | BCL11A | 200 | 96 | 0.48 |
| | | KDM6B | 159 | 76 | 0.477987421 |
| | | HBE1 | 3310 | 1564 | 0.472507553 |
| | | CBFA2T3 | 119 | 55 | 0.462184874 |
| | | LY86-AS1 | 53 | 24 | 0.452830189 |
| | | PLCG2 | 30 | 13 | 0.433333333 |
| | | STAT5A | 4961 | 2103 | 0.42390647 |
| | | LAPTM5 | 31 | 13 | 0.419354839 |
| | | NUP210 | 142 | 57 | 0.401408451 |
| | | MIR144 | 32 | 12 | 0.375 |
| | | GDPD5 | 16 | 6 | 0.375 |
| | | IKZF1 | 1278 | 469 | 0.366979656 |
| | | FADS2 | 264 | 95 | 0.359848485 |
| | | IER2 | 31 | 11 | 0.35483871 |
| | | SIGLEC6 | 17 | 6 | 0.352941176 |
| | | SPTA1 | 1778 | 614 | 0.345331834 |
| | | SRSF5 | 18292 | 6316 | 0.345287557 |
| | | ZFP36 | 9123 | 3089 | 0.33859476 |
| | | MIDN | 15 | 5 | 0.333333333 |
| | | FAM38A | 9 | 3 | 0.333333333 |
| | | CIC | 3500 | 1151 | 0.328857143 |
| | | ID2 | 836 | 269 | 0.321770335 |
| | | KLF13 | 50 | 16 | 0.32 |
| | | ABCC4 | 613 | 188 | 0.306688418 |
| | | RIN3 | 10 | 3 | 0.3 |
| | | CCND3 | 580 | 171 | 0.294827586 |
| | | TET3 | 65 | 19 | 0.292307692 |
| | | NPRL3 | 63153 | 18370 | 0.290880877 |
| | | ST8SIA6 | 7 | 2 | 0.285714286 |
| | | JARID2 | 121 | 33 | 0.272727273 |
| | | IFITM1 | 2776 | 736 | 0.265129683 |
| | | SPTB | 522 | 138 | 0.264367816 |
| | | CD82 | 33053 | 8731 | 0.264151514 |
| | | TNFAIP8 | 57 | 15 | 0.263157895 |
| | | EMP3 | 84 | 22 | 0.261904762 |
| | | PIM1 | 1895 | 495 | 0.26121372 |
| | | MLL2 | 161 | 42 | 0.260869565 |
| | | HAGH | 95 | 24 | 0.252631579 |
| CD34_fetal | [‘TAL1’, | GFI1B | 72 | 54 | 0.75 |
| | ‘STAT5A’, | CD58 | 1619 | 1126 | 0.695491044 |
| | ‘IKZF1’, | TMEM56 | 3 | 2 | 0.666666667 |
| | ‘NFE2’]103 | LRRC8D | 3 | 2 | 0.666666667 |
| | | LMO2 | 440 | 273 | 0.620454545 |
| | | SLC25A37 | 12163 | 7342 | 0.603633972 |
| | | LYL1 | 65 | 39 | 0.6 |
| | | TM9SF3 | 49 | 28 | 0.571428571 |
| | | RHD | 2342 | 1272 | 0.543125534 |
| | | SH2D4B | 2 | 1 | 0.5 |
| | | LGALS9 | 212 | 106 | 0.5 |
| | | HBE1 | 3310 | 1564 | 0.472507553 |
| | | FABP6 | 144128 | 65242 | 0.452667074 |
| | | STAT5A | 4961 | 2103 | 0.42390647 |
| | | FAM46C | 5 | 2 | 0.4 |
| | | GDPD5 | 16 | 6 | 0.375 |
| | | IKZF1 | 1278 | 469 | 0.366979656 |
| | | SIGLEC6 | 17 | 6 | 0.352941176 |
| | | MIDN | 15 | 5 | 0.333333333 |
| | | KLF13 | 50 | 16 | 0.32 |
| | | CCND3 | 580 | 171 | 0.294827586 |
| | | TET3 | 65 | 19 | 0.292307692 |
| | | NPRL3 | 63153 | 18370 | 0.290880877 |
| | | ST8SIA6 | 7 | 2 | 0.285714286 |
| | | HPS1 | 2669 | 757 | 0.283626827 |
| | | BMP2K | 8323 | 2265 | 0.27213745 |
| | | SPTB | 522 | 138 | 0.264367816 |
| | | PIM1 | 1895 | 495 | 0.26121372 |
| | | RREB1 | 350 | 87 | 0.248571429 |
| | | TAL1 | 5638 | 1361 | 0.241397659 |
| | | LDB1 | 300 | 71 | 0.236666667 |
| | | ANK1 | 827 | 190 | 0.22974607 |
| | | PIK3R1 | 2665 | 588 | 0.220637899 |
| | | CPEB4 | 23 | 5 | 0.217391304 |
| | | KIAA0040 | 5 | 1 | 0.2 |
| | | TRAK2 | 93 | 18 | 0.193548387 |
| | | SH3GL1 | 186 | 36 | 0.193548387 |
| | | SLC4A1 | 5092562 | 983895 | 0.193202361 |
| | | FECH | 2134 | 408 | 0.191190253 |
| | | ARL4A | 21 | 4 | 0.19047619 |
| | | GYPC | 2604384 | 483868 | 0.185789807 |
| | | GATA5 | 184 | 34 | 0.184782609 |
| | | JUNB | 15304 | 2825 | 0.184592263 |
| | | NEAT1 | 117 | 21 | 0.179487179 |
| | | KLF9 | 140 | 25 | 0.178571429 |
| | | NFE2 | 4177 | 743 | 0.17787886 |
| | | MIR101-2 | 42 | 7 | 0.166666667 |
| | | NOX5 | 140 | 23 | 0.164285714 |
| | | EED | 1039 | 168 | 0.161693936 |
| | | TMBIM1 | 13 | 2 | 0.153846154 |
| CD56 | [‘ZBTB16’, | CCL3 | 3252 | 2439 | 0.75 |
| | ‘FLI1’, | CCL5 | 7504 | 4245 | 0.565698294 |
| | ‘SMAD3’, | SIGLEC9 | 61 | 31 | 0.508196721 |
| | ‘NR4A2’, | LRRC33 | 2 | 1 | 0.5 |
| | ‘IRF2’, | CX3CR1 | 1055 | 500 | 0.473933649 |
| | ‘TGIF1’]542 | ICAM2 | 316 | 141 | 0.446202532 |
| | | AOAH | 32 | 14 | 0.4375 |
| | | ITGB2 | 22607 | 9702 | 0.42915911 |
| | | CD97 | 152 | 65 | 0.427631579 |
| | | FCGR3B | 6753 | 2878 | 0.426180957 |
| | | FCGR3A | 6819 | 2882 | 0.422642616 |
| | | CD53 | 152 | 63 | 0.414473684 |
| | | IRAK2 | 993 | 355 | 0.357502518 |
| | | CCR7 | 2514 | 892 | 0.354813047 |
| | | CD300A | 56 | 19 | 0.339285714 |
| | | PILRB | 12 | 4 | 0.333333333 |
| | | C20orf3 | 3 | 1 | 0.333333333 |
| | | CCR6 | 1258 | 415 | 0.329888712 |
| | | TBCC | 2718 | 871 | 0.320456218 |
| | | IL16 | 733 | 228 | 0.311050477 |
| | | CMKLR1 | 217 | 65 | 0.299539171 |
| | | LY9 | 69 | 20 | 0.289855072 |
| | | CD58 | 1619 | 468 | 0.289067326 |
| | | LRRC8A | 7 | 2 | 0.285714286 |
| | | LCP2 | 495 | 141 | 0.284848485 |
| | | IL10RA | 166 | 47 | 0.28313253 |
| | | CTAGE1 | 233 | 65 | 0.278969957 |
| | | NLRC5 | 44 | 12 | 0.272727273 |
| | | GAB3 | 15 | 4 | 0.266666667 |
| | | LBR | 18340 | 4657 | 0.253925845 |
| | | PTPRC | 17928 | 4514 | 0.251784917 |
| | | KIAA0247 | 4 | 1 | 0.25 |
| | | GPR183 | 38 | 9 | 0.236842105 |
| | | ZC3H12A | 268 | 62 | 0.231343284 |
| | | LPXN | 26 | 6 | 0.230769231 |
| | | ARL4C | 3420 | 785 | 0.229532164 |
| | | CLEC2D | 59 | 13 | 0.220338983 |
| | | CXCR4 | 9055 | 1987 | 0.219436775 |
| | | IFNAR2 | 2107 | 458 | 0.217370669 |
| | | HLA-C | 2739 | 595 | 0.217232567 |
| | | FMNL1 | 43 | 9 | 0.209302326 |
| | | STK4 | 345 | 72 | 0.208695652 |
| | | KLRD1 | 867 | 179 | 0.206459054 |
| | | IL17C | 6891 | 1416 | 0.205485416 |
| | | CXCR5 | 600 | 123 | 0.205 |
| | | HLA-DRB1 | 8174 | 1656 | 0.202593589 |
| | | XCL2 | 20 | 4 | 0.2 |
| | | GLIPR2 | 15 | 3 | 0.2 |
| | | ISG20 | 13861 | 2765 | 0.199480557 |
| | | CEACAM21 | 58 | 11 | 0.189655172 |
| CD8_primary | [‘BACH2’, | PHF15 | 1 | 1 | 1 |
| | ‘FLI1’, | ISG20 | 13861 | 13066 | 0.942644831 |
| | ‘SMAD3’, | CRTAM | 32 | 30 | 0.9375 |
| | ‘IKZF1’, | CD247 | 429 | 386 | 0.8997669 |
| | ‘NR4A2’, | TBX21 | 1698 | 1490 | 0.877502945 |
| | ‘STAT5B’, | IL7R | 2780 | 2436 | 0.876258993 |
| | ‘SREBF1’, | LCK | 3367 | 2863 | 0.85031185 |
| | ‘TGIF1’]582 | IL2RB | 1371 | 1155 | 0.842450766 |
| | | CCR7 | 2514 | 2064 | 0.821002387 |
| | | NFATC2 | 496 | 406 | 0.818548387 |
| | | LCP2 | 495 | 399 | 0.806060606 |
| | | CD84 | 71 | 57 | 0.802816901 |
| | | SKAP1 | 55 | 44 | 0.8 |
| | | NLRC5 | 44 | 34 | 0.772727273 |
| | | KLRK1 | 1692 | 1294 | 0.764775414 |
| | | TCF7 | 343 | 258 | 0.752186589 |
| | | GVINP1 | 8 | 6 | 0.75 |
| | | CD6 | 407 | 300 | 0.737100737 |
| | | KLRD1 | 867 | 630 | 0.726643599 |
| | | NFATC3 | 215 | 153 | 0.711627907 |
| | | ARL4C | 3420 | 2399 | 0.701461988 |
| | | GIMAP5 | 74 | 51 | 0.689189189 |
| | | FCGR3B | 6753 | 4537 | 0.671849548 |
| | | FCGR3A | 6819 | 4551 | 0.667399912 |
| | | ZC3HAV1 | 2531 | 1685 | 0.665744765 |
| | | CD53 | 152 | 101 | 0.664473684 |
| | | BTN3A2 | 14 | 9 | 0.642857143 |
| | | MYADM | 11 | 7 | 0.636363636 |
| | | STAT4 | 1031 | 656 | 0.636275461 |
| | | PRKCQ | 404 | 257 | 0.636138614 |
| | | BATF | 95 | 60 | 0.631578947 |
| | | GZMH | 46 | 28 | 0.608695652 |
| | | CD3D | 332 | 199 | 0.59939759 |
| | | CD8A | 118848 | 71224 | 0.599286484 |
| | | CCL5 | 7504 | 4375 | 0.583022388 |
| | | IFNAR2 | 2107 | 1150 | 0.545799715 |
| | | SIRPG | 17 | 9 | 0.529411765 |
| | | CXCR6 | 353 | 185 | 0.52407932 |
| | | CD2 | 16582 | 8576 | 0.517187312 |
| | | PTPRC | 17928 | 9197 | 0.51299643 |
| | | IL10RA | 166 | 85 | 0.512048193 |
| | | FASLG | 10454 | 5233 | 0.500573943 |
| | | PILRB | 12 | 6 | 0.5 |
| | | KIAA0922 | 2 | 1 | 0.5 |
| | | DOCK8 | 90 | 45 | 0.5 |
| | | TAP1 | 1353 | 670 | 0.495195861 |
| | | CLEC2D | 59 | 29 | 0.491525424 |
| | | IL16 | 733 | 348 | 0.474761255 |
| | | BCL6 | 1505 | 709 | 0.471096346 |
| | | PLCG2 | 30 | 14 | 0.466666667 |
| Colon_Crypt_1 | [‘NR4A1’, | KIF26A | 1 | 1 | 1 |
| | ‘SMAD3’, | CDHR2 | 6 | 3 | 0.5 |
| | ‘FOXA1’, | B3GALT5 | 23 | 8 | 0.347826087 |
| | ‘HES1’, | SHROOM1 | 3 | 1 | 0.333333333 |
| | ‘RREB1’, | AIFM3 | 4 | 1 | 0.25 |
| | ‘ELF3’, | CDX1 | 240 | 55 | 0.229166667 |
| | ‘SREBF1’, | B3GNT7 | 9 | 2 | 0.222222222 |
| | ‘FOXP1’, | AFAP1 | 115 | 23 | 0.2 |
| | ‘SREBF2’, | RNF43 | 55 | 10 | 0.181818182 |
| | ‘KLF4’, | APOLD1 | 2453 | 390 | 0.158988993 |
| | ‘TGIF1’, | RXFP4 | 48 | 7 | 0.145833333 |
| | ‘NR4A2’, | CDX2 | 1304 | 185 | 0.141871166 |
| | ‘ATF3’]538 | FXYD3 | 60 | 8 | 0.133333333 |
| | | GPRC5C | 8 | 1 | 0.125 |
| | | B3GNT8 | 8 | 1 | 0.125 |
| | | TCF7L2 | 1739 | 217 | 0.124784359 |
| | | MUC2 | 3072 | 373 | 0.121419271 |
| | | FAM3D | 25 | 3 | 0.12 |
| | | GCNT3 | 17 | 2 | 0.117647059 |
| | | SLC16A5 | 19 | 2 | 0.105263158 |
| | | SLC9A8 | 43 | 4 | 0.093023256 |
| | | DUOX2 | 172 | 16 | 0.093023256 |
| | | SPIRE2 | 11 | 1 | 0.090909091 |
| | | KRT80 | 11 | 1 | 0.090909091 |
| | | HIC1 | 226 | 18 | 0.079646018 |
| | | TMPRSS4 | 103 | 8 | 0.077669903 |
| | | SIGIRR | 91 | 7 | 0.076923077 |
| | | MUC12 | 390 | 30 | 0.076923077 |
| | | KLF5 | 348 | 24 | 0.068965517 |
| | | ZNF217 | 102 | 7 | 0.068627451 |
| | | MIR145 | 481 | 33 | 0.068607069 |
| | | FZD5 | 88 | 6 | 0.068181818 |
| | | CSRNP1 | 15 | 1 | 0.066666667 |
| | | MUC4 | 876 | 57 | 0.065068493 |
| | | ATP2C2 | 31 | 2 | 0.064516129 |
| | | CDC42EP4 | 16 | 1 | 0.0625 |
| | | PDLIM1 | 51 | 3 | 0.058823529 |
| | | MLKL | 34 | 2 | 0.058823529 |
| | | MMP23A | 36 | 2 | 0.055555556 |
| | | ATP1B1 | 92 | 5 | 0.054347826 |
| | | PIM3 | 131 | 7 | 0.053435115 |
| | | CCBP2 | 19 | 1 | 0.052631579 |
| | | ATP2A3 | 134 | 7 | 0.052238806 |
| | | PIGR | 350 | 18 | 0.051428571 |
| | | MIR200C | 20 | 1 | 0.05 |
| | | KLF4 | 1466 | 71 | 0.048431105 |
| | | GPRC5A | 43 | 2 | 0.046511628 |
| | | FABP1 | 645 | 30 | 0.046511628 |
| | | SFN | 830 | 37 | 0.044578313 |
| | | RXRA | 115 | 5 | 0.043478261 |
| Colon_Crypt_2 | [‘FOXP1’, | KIF26A | 1 | 1 | 1 |
| | ‘IRF1’, | SMAGP | 3 | 2 | 0.666666667 |
| | ‘FOXA1’, | CDHR2 | 6 | 3 | 0.5 |
| | ‘ZNF219’, | LDHD | 1300 | 583 | 0.448461538 |
| | ‘GTF2IRD1’, | AIFM3 | 4 | 1 | 0.25 |
| | ‘KLF4’, | CDX1 | 240 | 55 | 0.229166667 |
| | ‘SREBF2’, | DENND2D | 5 | 1 | 0.2 |
| | ‘SREBF1’, | AFAP1 | 115 | 23 | 0.2 |
| | ‘NR5A2’, | APOLD1 | 2453 | 390 | 0.158988993 |
| | ‘HES1’, | RXFP4 | 48 | 7 | 0.145833333 |
| | ‘KLF12’, | GAL3ST2 | 21 | 3 | 0.142857143 |
| | ‘SMAD3’, | CDX2 | 1304 | 185 | 0.141871166 |
| | ‘NR4A2’, | BCL9L | 29 | 4 | 0.137931034 |
| | ‘ELF3’, | FXYD3 | 60 | 8 | 0.133333333 |
| | ‘NR4A1’, | MUC2 | 3072 | 373 | 0.121419271 |
| | ‘TGIF1’]610 | FAM3D | 25 | 3 | 0.12 |
| | | MIR26A1 | 9 | 1 | 0.111111111 |
| | | ACTN1 | 55 | 6 | 0.109090909 |
| | | SLC16A5 | 19 | 2 | 0.105263158 |
| | | MBOAT7 | 284 | 28 | 0.098591549 |
| | | DUOX2 | 172 | 16 | 0.093023256 |
| | | SPIRE2 | 11 | 1 | 0.090909091 |
| | | HIC1 | 226 | 18 | 0.079646018 |
| | | SIGIRR | 91 | 7 | 0.076923077 |
| | | MUC12 | 390 | 30 | 0.076923077 |
| | | MIR145 | 481 | 33 | 0.068607069 |
| | | FZD5 | 88 | 6 | 0.068181818 |
| | | CSRNP1 | 15 | 1 | 0.066666667 |
| | | MUC4 | 876 | 57 | 0.065068493 |
| | | ATP2C2 | 31 | 2 | 0.064516129 |
| | | TP53I11 | 16 | 1 | 0.0625 |
| | | CDC42EP4 | 16 | 1 | 0.0625 |
| | | PDLIM1 | 51 | 3 | 0.058823529 |
| | | MLKL | 34 | 2 | 0.058823529 |
| | | ABCC3 | 697 | 40 | 0.057388809 |
| | | MMP23A | 36 | 2 | 0.055555556 |
| | | ATP1B1 | 92 | 5 | 0.054347826 |
| | | PIM3 | 131 | 7 | 0.053435115 |
| | | PIK3IP1 | 38 | 2 | 0.052631579 |
| | | ATP2A3 | 134 | 7 | 0.052238806 |
| | | PIGR | 350 | 18 | 0.051428571 |
| | | S100A11 | 177 | 9 | 0.050847458 |
| | | MIR200C | 20 | 1 | 0.05 |
| | | IFITM3 | 122 | 6 | 0.049180328 |
| | | BIK | 615 | 30 | 0.048780488 |
| | | CCND1 | 14530 | 707 | 0.048657949 |
| | | KLF4 | 1466 | 71 | 0.048431105 |
| | | IER3 | 212 | 10 | 0.047169811 |
| | | FABP1 | 645 | 30 | 0.046511628 |
| | | SLCO2B1 | 240 | 11 | 0.045833333 |
| Colon_Crypt_3 | [‘FOXP1’, | CDHR2 | 6 | 3 | 0.5 |
| | ‘SREBF2’, | SHROOM1 | 3 | 1 | 0.333333333 |
| | ‘SREBF1’, | AIFM3 | 4 | 1 | 0.25 |
| | ‘KLF4’, | CDX1 | 240 | 55 | 0.229166667 |
| | ‘NR5A2’, | B3GNT7 | 9 | 2 | 0.222222222 |
| | ‘HES1’, | AFAP1 | 115 | 23 | 0.2 |
| | ‘NR4A2’, | CDX2 | 1304 | 185 | 0.141871166 |
| | ‘NR4A1’, | BCL9L | 29 | 4 | 0.137931034 |
| | ‘ELF3’, | GPRC5C | 8 | 1 | 0.125 |
| | ‘TGIF1’, | MUC2 | 3072 | 373 | 0.121419271 |
| | ‘FOXA1’]368 | SPIRE2 | 11 | 1 | 0.090909091 |
| | | SLC9A3 | 917 | 75 | 0.081788441 |
| | | SIGIRR | 91 | 7 | 0.076923077 |
| | | OPLAH | 39 | 3 | 0.076923077 |
| | | MUC12 | 390 | 30 | 0.076923077 |
| | | KLF5 | 348 | 24 | 0.068965517 |
| | | CLDN7 | 1267 | 87 | 0.06866614 |
| | | FZD5 | 88 | 6 | 0.068181818 |
| | | CSRNP1 | 15 | 1 | 0.066666667 |
| | | MUC4 | 876 | 57 | 0.065068493 |
| | | CDC42EP4 | 16 | 1 | 0.0625 |
| | | PDLIM1 | 51 | 3 | 0.058823529 |
| | | MMP23A | 36 | 2 | 0.055555556 |
| | | ATP1B1 | 92 | 5 | 0.054347826 |
| | | PIM3 | 131 | 7 | 0.053435115 |
| | | CCBP2 | 19 | 1 | 0.052631579 |
| | | ATP2A3 | 134 | 7 | 0.052238806 |
| | | MIR200C | 20 | 1 | 0.05 |
| | | KLF4 | 1466 | 71 | 0.048431105 |
| | | CBR3 | 68 | 3 | 0.044117647 |
| | | RXRA | 115 | 5 | 0.043478261 |
| | | MUC5B | 829 | 36 | 0.043425814 |
| | | SCNN1A | 168 | 7 | 0.041666667 |
| | | CDKN1A | 29540 | 1205 | 0.040792146 |
| | | SLC22A5 | 517 | 21 | 0.040618956 |
| | | ITGB4 | 850 | 33 | 0.038823529 |
| | | PTPRK | 336 | 13 | 0.038690476 |
| | | LY86-AS1 | 53 | 2 | 0.037735849 |
| | | TACC2 | 27 | 1 | 0.037037037 |
| | | RHOU | 83 | 3 | 0.036144578 |
| | | ITPKC | 28 | 1 | 0.035714286 |
| | | SLCO4A1 | 312 | 11 | 0.03525641 |
| | | MGAT4A | 57 | 2 | 0.035087719 |
| | | EPCAM | 5214 | 182 | 0.034906022 |
| | | PITPNA | 29 | 1 | 0.034482759 |
| | | LGALS3 | 2524 | 87 | 0.034469097 |
| | | HRC | 1107 | 35 | 0.031616983 |
| | | CDKN1B | 7412 | 230 | 0.031030761 |
| | | PTPRF | 2325 | 71 | 0.030537634 |
| | | HSD11B2 | 1843 | 53 | 0.028757461 |
| H1 | [‘SOX2’, | ZSCAN10 | 6 | 5 | 0.833333333 |
| | ‘GTF2I’, | DPPA4 | 25 | 19 | 0.76 |
| | ‘FOXD3’, | NANOG | 2608 | 1775 | 0.68059816 |
| | ‘MYB’, | POU5F1 | 6308 | 3188 | 0.505389981 |
| | ‘POU5F1’, | GRAMD3 | 2 | 1 | 0.5 |
| | ‘NR5A1’, | SOX2 | 3476 | 1657 | 0.476697353 |
| | ‘NANOG’]352 | LIN28A | 428 | 182 | 0.425233645 |
| | | AKR1D1 | 33 | 12 | 0.363636364 |
| | | ZNF462 | 9 | 3 | 0.333333333 |
| | | MIR302B | 3 | 1 | 0.333333333 |
| | | CYP2S1 | 56 | 18 | 0.321428571 |
| | | JARID2 | 121 | 33 | 0.272727273 |
| | | DAZL | 292 | 69 | 0.23630137 |
| | | AEBP2 | 13 | 3 | 0.230769231 |
| | | KDM2B | 41 | 9 | 0.219512195 |
| | | SALL4 | 427 | 88 | 0.206088993 |
| | | LIN28B | 121 | 24 | 0.198347107 |
| | | SETD1B | 26 | 5 | 0.192307692 |
| | | USP44 | 12 | 2 | 0.166666667 |
| | | RAI14 | 12 | 2 | 0.166666667 |
| | | ODZ2 | 6 | 1 | 0.166666667 |
| | | LRRK1 | 28 | 4 | 0.142857143 |
| | | TRIM71 | 63 | 8 | 0.126984127 |
| | | TGIF2LX | 8 | 1 | 0.125 |
| | | TEAD3 | 40. | 5 | 0.125 |
| | | SOX21 | 41 | 5 | 0.12195122 |
| | | MIR106A | 17 | 2 | 0.117647059 |
| | | CECR2 | 17 | 2 | 0.117647059 |
| | | INSC | 122 | 14 | 0.114754098 |
| | | GYLTL1B | 9 | 1 | 0.111111111 |
| | | TNRC6B | 19 | 2 | 0.105263158 |
| | | PHF17 | 19 | 2 | 0.105263158 |
| | | BCL11A | 200 | 21 | 0.105 |
| | | ZNF281 | 10 | 1 | 0.1 |
| | | SALL2 | 32 | 3 | 0.09375 |
| | | IDO2 | 54 | 5 | 0.092592593 |
| | | ZMYND8 | 11 | 1 | 0.090909091 |
| | | PHC1 | 121 | 11 | 0.090909091 |
| | | SOX11 | 298 | 27 | 0.090604027 |
| | | FZD7 | 146 | 13 | 0.089041096 |
| | | USP28 | 24 | 2 | 0.083333333 |
| | | FOXN3 | 36 | 3 | 0.083333333 |
| | | LDB2 | 182 | 14 | 0.076923077 |
| | | HIST1H4I | 13 | 1 | 0.076923077 |
| | | CGNL1 | 13 | 1 | 0.076923077 |
| | | BCOR | 109 | 8 | 0.073394495 |
| | | CDH8 | 57 | 4 | 0.070175439 |
| | | SOX13 | 44 | 3 | 0.068181818 |
| | | ITGB1 | 5414 | 369 | 0.068156631 |
| | | PPAP2B | 61 | 4 | 0.06557377 |
| HMEC | [‘TFCP2L1’, | MIR661 | 2 | 2 | 1 |
| | ‘NEUROD1’, | MAGEF1 | 1 | 1 | 1 |
| | ‘SMAD3’, | FLJ43663 | 1 | 1 | 1 |
| | ‘KLF4’, | FAM83B | 5 | 4 | 0.8 |
| | ‘TGIF1’, | RNF152 | 3 | 1 | 0.333333333 |
| | ‘NR4A2’, | CITED4 | 12 | 4 | 0.333333333 |
| | ‘HES1’, | RAD51L1 | 47 | 15 | 0.319148936 |
| | ‘HOXA5’, | TRIM16 | 21 | 6 | 0.285714286 |
| | ‘SREBF1’, | KRT80 | 11 | 3 | 0.272727273 |
| | ‘HIF1A’]612 | POU5F1B | 15 | 4 | 0.266666667 |
| | | EGFR | 67027 | 17169 | 0.256150507 |
| | | IRF2BP2 | 12 | 3 | 0.25 |
| | | TNS4 | 31 | 7 | 0.225806452 |
| | | TNKS1BP1 | 5 | 1 | 0.2 |
| | | SLC22A23 | 5 | 1 | 0.2 |
| | | LIMA1 | 32 | 6 | 0.1875 |
| | | HSD17B2 | 1797 | 330 | 0.183639399 |
| | | PLEKHG6 | 11 | 2 | 0.181818182 |
| | | SLCO3A1 | 45 | 8 | 0.177777778 |
| | | SSPN | 725 | 120 | 0.165517241 |
| | | SUMO1P1 | 7 | 1 | 0.142857143 |
| | | PPP4R1 | 7 | 1 | 0.142857143 |
| | | GPRC5A | 43 | 6 | 0.139534884 |
| | | MYOF | 37 | 5 | 0.135135135 |
| | | TBX3 | 570 | 76 | 0.133333333 |
| | | PARD6B | 15 | 2 | 0.133333333 |
| | | CCNG2 | 61 | 8 | 0.131147541 |
| | | DFNA5 | 54 | 7 | 0.12962963 |
| | | FGFBP1 | 93 | 12 | 0.129032258 |
| | | SNX9 | 256 | 32 | 0.125 |
| | | ARHGAP12 | 8 | 1 | 0.125 |
| | | PHLDA1 | 82 | 10 | 0.12195122 |
| | | S100A16 | 17 | 2 | 0.117647059 |
| | | SEC14L1 | 18 | 2 | 0.111111111 |
| | | RNF19B | 9 | 1 | 0.111111111 |
| | | ARTN | 918 | 99 | 0.107843137 |
| | | TPM4 | 47 | 5 | 0.106382979 |
| | | MIR21 | 1479 | 154 | 0.104124408 |
| | | TRPS1 | 154 | 16 | 0.103896104 |
| | | VEGFC | 1849 | 190 | 0.102758248 |
| | | ETS2 | 435 | 44 | 0.101149425 |
| | | ITGA6 | 1908 | 192 | 0.100628931 |
| | | HOXA5 | 249 | 25 | 0.100401606 |
| | | MMP14 | 2594 | 260 | 0.100231303 |
| | | TFCP2L1 | 20 | 2 | 0.1 |
| | | RTKN | 40 | 4 | 0.1 |
| | | S100A2 | 192 | 19 | 0.098958333 |
| | | CDKN1B | 7412 | 727 | 0.098084188 |
| | | MIR222 | 328 | 32 | 0.097560976 |
| | | PRICKLE2 | 31 | 3 | 0.096774194 |
| NHDF-Ad | [‘NR4A1’, | MIR1205 | 4 | 3 | 0.75 |
| | ‘KLF4’, | COL6A2 | 110 | 42 | 0.381818182 |
| | ‘TGIF1’, | KLF4 | 1466 | 528 | 0.360163711 |
| | ‘SREBF1’, | GRLF1 | 112 | 40 | 0.357142857 |
| | ‘HIF1A’]490 | MED15 | 222 | 78 | 0.351351351 |
| | | SDC4 | 539 | 176 | 0.326530612 |
| | | IER2 | 31 | 10 | 0.322580645 |
| | | COL6A3 | 104 | 33 | 0.317307692 |
| | | COL1A1 | 1398 | 437 | 0.312589413 |
| | | PDGFRB | 9477 | 2605 | 0.274876016 |
| | | TWIST2 | 119 | 32 | 0.268907563 |
| | | HAS2-AS1 | 461 | 123 | 0.26681128 |
| | | PKIG | 12 | 3 | 0.25 |
| | | PITPNB | 16 | 4 | 0.25 |
| | | MRPS22 | 16 | 4 | 0.25 |
| | | METRNL | 4 | 1 | 0.25 |
| | | LAYN | 4 | 1 | 0.25 |
| | | C11orf59 | 4 | 1 | 0.25 |
| | | FBLN1 | 50 | 12 | 0.24 |
| | | PHLDA1 | 82 | 19 | 0.231707317 |
| | | SH3PXD2B | 26 | 6 | 0.230769231 |
| | | VGLL4 | 9 | 2 | 0.222222222 |
| | | LTBP2 | 117 | 26 | 0.222222222 |
| | | OSR2 | 42 | 9 | 0.214285714 |
| | | ADAMTSL1 | 14 | 3 | 0.214285714 |
| | | BCL9L | 29 | 6 | 0.206896552 |
| | | HSP90B3P | 5 | 1 | 0.2 |
| | | SMAD3 | 3407 | 664 | 0.194892868 |
| | | CYR61 | 646 | 125 | 0.193498452 |
| | | RFX2 | 32 | 6 | 0.1875 |
| | | CDC42EP4 | 16 | 3 | 0.1875 |
| | | ADAMTS14 | 16 | 3 | 0.1875 |
| | | EPAS1 | 789 | 146 | 0.18504436 |
| | | SMAD7 | 1310 | 233 | 0.177862595 |
| | | ITGB1 | 5414 | 935 | 0.172700406 |
| | | MLLT1 | 643 | 110 | 0.171073095 |
| | | MMP14 | 2594 | 435 | 0.16769468 |
| | | SMAD6 | 1367 | 228 | 0.166788588 |
| | | RASSF8 | 12 | 2 | 0.166666667 |
| | | RASSF10 | 18 | 3 | 0.166666667 |
| | | ERGIC1 | 6 | 1 | 0.166666667 |
| | | ARHGEF17 | 12 | 2 | 0.166666667 |
| | | CREB3L2 | 55 | 9 | 0.163636364 |
| | | PXN | 817 | 131 | 0.160342717 |
| | | SPARC | 2584 | 414 | 0.160216718 |
| | | SERTAD1 | 39 | 6 | 0.153846154 |
| | | FOSL2 | 260 | 40 | 0.153846154 |
| | | TGFBR1 | 1066 | 154 | 0.144465291 |
| | | CSNK1A1 | 573 | 80 | 0.139616056 |
| | | EMX2 | 205 | 27 | 0.131707317 |
| NHLF | [‘SMAD3’, | CT62 | 1 | 1 | 1 |
| | ‘RREB1’, | C8orf46 | 1 | 1 | 1 |
| | ‘KLF4’, | CALU | 995 | 595 | 0.59798995 |
| | ‘NR4A2’, | LOC554202 | 2 | 1 | 0.5 |
| | ‘ARID5B’, | ARHGAP23 | 3 | 1 | 0.333333333 |
| | ‘NR4A1’]521 | ITGB6 | 29 | 9 | 0.310344828 |
| | | VGLL4 | 9 | 2 | 0.222222222 |
| | | PCID2 | 1940 | 425 | 0.219072165 |
| | | WHSC1L1 | 30 | 6 | 0.2 |
| | | HS3ST3A1 | 5 | 1 | 0.2 |
| | | CSRNP1 | 15 | 3 | 0.2 |
| | | NTM | 1787 | 339 | 0.189703414 |
| | | ADAMTS6 | 16 | 3 | 0.1875 |
| | | DBN1 | 11 | 2 | 0.181818182 |
| | | HDGF | 131 | 23 | 0.175572519 |
| | | UACA | 24 | 4 | 0.166666667 |
| | | MED15 | 222 | 37 | 0.166666667 |
| | | ARHGEF17 | 12 | 2 | 0.166666667 |
| | | KLF2 | 351 | 57 | 0.162393162 |
| | | SASH1 | 19 | 3 | 0.157894737 |
| | | S100A2 | 192 | 27 | 0.140625 |
| | | TMSB10 | 107 | 15 | 0.140186916 |
| | | EGFR | 67027 | 8869 | 0.132319811 |
| | | SPRY2 | 281 | 37 | 0.131672598 |
| | | ABCC1 | 5571 | 651 | 0.116855143 |
| | | LTBP1 | 131 | 15 | 0.114503817 |
| | | SPATS2L | 18 | 2 | 0.111111111 |
| | | LTBP2 | 117 | 13 | 0.111111111 |
| | | FAM38A | 9 | 1 | 0.111111111 |
| | | LOXL2 | 118 | 13 | 0.110169492 |
| | | GNA12 | 3484 | 377 | 0.108208955 |
| | | TPM4 | 47 | 5 | 0.106382979 |
| | | FOXL1 | 58 | 6 | 0.103448276 |
| | | PDGFC | 155 | 16 | 0.103225806 |
| | | CTGF | 2796 | 276 | 0.098712446 |
| | | VEGFC | 1849 | 180 | 0.097349919 |
| | | ERRFI1 | 226 | 22 | 0.097345133 |
| | | EPHA2 | 2474 | 235 | 0.094987874 |
| | | SMAD3 | 3407 | 322 | 0.0945113 |
| | | STK40 | 194 | 18 | 0.092783505 |
| | | TWIST2 | 119 | 11 | 0.092436975 |
| | | MIR21 | 1479 | 135 | 0.09127789 |
| | | KCTD10 | 11 | 1 | 0.090909091 |
| | | NFIX | 56 | 5 | 0.089285714 |
| | | ECT2 | 140 | 12 | 0.085714286 |
| | | SPRY4 | 119 | 10 | 0.084033613 |
| | | SH2D4A | 12 | 1 | 0.083333333 |
| | | RAI14 | 12 | 1 | 0.083333333 |
| | | NEURL | 12 | 1 | 0.083333333 |
| | | IRF2BP2 | 12 | 1 | 0.083333333 |
| Skeletal_Muscle_Myoblast | [‘GLIS3’, | ASB7 | 1 | 1 | 1 |
| | ‘TGIF1’, | MYF6 | 437 | 414 | 0.947368421 |
| | ‘RREB1’, | MEF2D | 168 | 126 | 0.75 |
| | ‘KLF12’, | MYOF | 37 | 27 | 0.72972973 |
| | ‘ZBTB16’, | TRIM55 | 31 | 22 | 0.709677419 |
| | ‘FOSL1’]470 | RBM24 | 10 | 7 | 0.7 |
| | | CHRNA1 | 507 | 321 | 0.633136095 |
| | | LMCD1 | 13 | 8 | 0.615384615 |
| | | VGLL4 | 9 | 5 | 0.555555556 |
| | | TRIM43 | 2 | 1 | 0.5 |
| | | LRTM1 | 2 | 1 | 0.5 |
| | | SLC8A1 | 630 | 303 | 0.480952381 |
| | | ACTC1 | 122 | 51 | 0.418032787 |
| | | ADAM19 | 84 | 30 | 0.357142857 |
| | | ACTN1 | 55 | 18 | 0.327272727 |
| | | IRS1 | 2857 | 845 | 0.295764788 |
| | | CAPN2 | 115 | 34 | 0.295652174 |
| | | AFAP1-AS1 | 7 | 2 | 0.285714286 |
| | | ADAMTSL1 | 14 | 4 | 0.285714286 |
| | | CELF2 | 95 | 26 | 0.273684211 |
| | | AHNAK | 95 | 26 | 0.273684211 |
| | | ATOH8 | 15 | 4 | 0.266666667 |
| | | VGLL3 | 12 | 3 | 0.25 |
| | | PTCD2 | 4 | 1 | 0.25 |
| | | MRPL33 | 4 | 1 | 0.25 |
| | | MICAL2 | 8 | 2 | 0.25 |
| | | LMNA | 23436 | 5703 | 0.243343574 |
| | | PFKP | 42 | 10 | 0.238095238 |
| | | MYO1E | 105 | 25 | 0.238095238 |
| | | JPH2 | 173 | 39 | 0.225433526 |
| | | SIX1 | 371 | 80 | 0.215633423 |
| | | ADAM12 | 285 | 61 | 0.214035088 |
| | | IRS2 | 1446 | 307 | 0.21230982 |
| | | PDGFC | 155 | 32 | 0.206451613 |
| | | FHL2 | 989 | 190 | 0.192113246 |
| | | PHLDB2 | 16 | 3 | 0.1875 |
| | | GAPDH | 9338 | 1582 | 0.169415292 |
| | | FOXO3 | 1586 | 265 | 0.167087011 |
| | | PRSS23 | 12 | 2 | 0.166666667 |
| | | MYO18B | 18 | 3 | 0.166666667 |
| | | IRF2BP2 | 12 | 2 | 0.166666667 |
| | | SMAD3 | 3407 | 531 | 0.155855591 |
| | | MIR23B | 40 | 6 | 0.15 |
| | | LIMS1 | 4803 | 717 | 0.149281699 |
| | | NUAK1 | 61 | 9 | 0.147540984 |
| | | SDC4 | 539 | 79 | 0.146567718 |
| | | ID3 | 542 | 78 | 0.143911439 |
| | | CAV1 | 5940 | 854 | 0.143771044 |
| | | VAMP3 | 446 | 64 | 0.143497758 |
| | | IQGAP1 | 1745 | 250 | 0.143266476 |
| UCSD_Adrenal_Gland | [‘SREBF2’, | CYP11B2 | 1604 | 649 | 0.404613466 |
| | ‘SREBF1’, | CBLN3 | 11 | 2 | 0.181818182 |
| | ‘RREB1’, | ERGIC1 | 6 | 1 | 0.166666667 |
| | ‘DBP’, | NR5A1 | 5913 | 799 | 0.135125994 |
| | ‘NR4A1’, | CHST3 | 5360 | 590 | 0.110074627 |
| | ‘NR4A2’, | RPH3AL | 42 | 4 | 0.095238095 |
| | ‘HIF1A’, | COMT | 3502 | 319 | 0.091090805 |
| | ‘TGIF1’, | CDC42EP4 | 16 | 1 | 0.0625 |
| | ‘NR5A1’, | ABLIM1 | 32 | 2 | 0.0625 |
| | ‘ATF4’, | TNS1 | 850 | 53 | 0.062352941 |
| | ‘ZBTB16’]425 | CTDSP2 | 271 | 16 | 0.05904059 |
| | | ZCCHC14 | 17 | 1 | 0.058823529 |
| | | PDE8A | 51 | 3 | 0.058823529 |
| | | SCARB1 | 2019 | 109 | 0.053987122 |
| | | NR4A2 | 890 | 48 | 0.053932584 |
| | | FOSL2 | 260 | 12 | 0.046153846 |
| | | NR2F1 | 488 | 22 | 0.045081967 |
| | | SLC23A2 | 179 | 8 | 0.044692737 |
| | | CMIP | 23 | 1 | 0.043478261 |
| | | GATA6 | 527 | 22 | 0.041745731 |
| | | STAR | 13238 | 516 | 0.038978698 |
| | | NR2F2 | 473 | 16 | 0.033826638 |
| | | IER2 | 31 | 1 | 0.032258065 |
| | | NR4A1 | 3061 | 95 | 0.031035609 |
| | | C1QTNF1 | 2748 | 83 | 0.030203785 |
| | | MRAS | 305 | 9 | 0.029508197 |
| | | ST3GAL4 | 7289 | 215 | 0.029496502 |
| | | ARAP1 | 35 | 1 | 0.028571429 |
| | | DUSP1 | 1191 | 31 | 0.026028547 |
| | | INSR | 47446 | 1180 | 0.024870379 |
| | | ACTN4 | 3536 | 85 | 0.024038462 |
| | | DBP | 10189 | 223 | 0.021886348 |
| | | AHNAK | 95 | 2 | 0.021052632 |
| | | PBX1 | 579 | 12 | 0.020725389 |
| | | USP2 | 98 | 2 | 0.020408163 |
| | | IL6R | 11078 | 207 | 0.018685683 |
| | | ANKRD11 | 701 | 13 | 0.018544936 |
| | | SEMA4B | 57 | 1 | 0.01754386 |
| | | RXRA | 115 | 2 | 0.017391304 |
| | | B4GALT1 | 1787 | 31 | 0.01734751 |
| | | FAM129B | 93889 | 1607 | 0.017115956 |
| | | LMNA | 23436 | 399 | 0.01702509 |
| | | BHLHE40 | 296 | 5 | 0.016891892 |
| | | PAPD7 | 2963 | 49 | 0.016537293 |
| | | SH3BP5 | 5453901 | 88069 | 0.016147891 |
| | | KCNQ1 | 2424 | 39 | 0.016089109 |
| | | CORO1A | 1284 | 20 | 0.015576324 |
| | | AKR1B1 | 116533 | 1750 | 0.015017205 |
| | | TM7SF2 | 468 | 7 | 0.014957265 |
| | | FKBP5 | 6248 | 91 | 0.014884763 |
| UCSD_Aorta | [‘SP3’, | C15orf52 | 1 | 1 | 1 |
| | ‘NR4A1’, | LMNA | 23436 | 15173 | 0.647422768 |
| | ‘ZBTB16’, | PRDM6 | 6 | 3 | 0.5 |
| | ‘MEIS1’, | MRPL33 | 4 | 2 | 0.5 |
| | ‘SMAD3’, | C14orf4 | 2 | 1 | 0.5 |
| | ‘TCF7L2’, | C14orf179 | 2 | 1 | 0.5 |
| | ‘ARID5B’]542 | PYGB | 47 | 20 | 0.425531915 |
| | | PTGIS | 694 | 255 | 0.367435159 |
| | | ADRA1B | 9269 | 3401 | 0.366921998 |
| | | KLF2 | 351 | 125 | 0.356125356 |
| | | LDB3 | 1168 | 414 | 0.354452055 |
| | | PPP1R12B | 20 | 7 | 0.35 |
| | | ADSSL1 | 3 | 1 | 0.333333333 |
| | | KCNA5 | 1285 | 428 | 0.33307393 |
| | | PKDCC | 118 | 38 | 0.322033898 |
| | | SMTN | 96 | 30 | 0.3125 |
| | | PRKG1 | 166 | 51 | 0.307228916 |
| | | MEF2A | 1446 | 424 | 0.293222683 |
| | | RAMP1 | 335 | 97 | 0.289552239 |
| | | GRK5 | 309 | 88 | 0.284789644 |
| | | NEDD9 | 511 | 143 | 0.279843444 |
| | | TEAD3 | 40 | 11 | 0.275 |
| | | THSD4 | 11 | 3 | 0.272727273 |
| | | KCTD10 | 11 | 3 | 0.272727273 |
| | | TPM1 | 243 | 66 | 0.271604938 |
| | | CSRP1 | 27376 | 7352 | 0.2685564 |
| | | GATA6 | 527 | 141 | 0.267552182 |
| | | MYH10 | 23 | 6 | 0.260869565 |
| | | PTTG1IP | 855 | 219 | 0.256140351 |
| | | SNX19 | 8 | 2 | 0.25 |
| | | MTSS1L | 4 | 1 | 0.25 |
| | | MFAP4 | 20 | 5 | 0.25 |
| | | B4GALNT3 | 4 | 1 | 0.25 |
| | | NAV1 | 2951 | 706 | 0.239240935 |
| | | MYLK | 4842 | 1134 | 0.234200743 |
| | | ROCK2 | 428 | 100 | 0.23364486 |
| | | ADCY5 | 213 | 48 | 0.225352113 |
| | | RGS3 | 112 | 25 | 0.223214286 |
| | | VGLL4 | 9 | 2 | 0.222222222 |
| | | MRVI1 | 45 | 10 | 0.222222222 |
| | | CPXM2 | 9 | 2 | 0.222222222 |
| | | FSTL1 | 622 | 138 | 0.221864952 |
| | | TPM4 | 47 | 10 | 0.212765957 |
| | | SERPINE1 | 20104 | 4130 | 0.205431755 |
| | | HDAC5 | 5139 | 1048 | 0.203930726 |
| | | HEY2 | 546 | 111 | 0.203296703 |
| | | HAND2 | 1276 | 258 | 0.202194357 |
| | | NUFIP1 | 15 | 3 | 0.2 |
| | | FEM1B | 65 | 13 | 0.2 |
| | | LBH | 61 | 12 | 0.196721311 |
| UCSD_Bladder | [‘NR4A2’, | CD9 | 1639 | 42 | 0.025625381 |
| | ‘SMAD3’, | TAGLN | 828 | 18 | 0.02173913 |
| | ‘SREBF1’, | TPM4 | 47 | 1 | 0.021276596 |
| | ‘TGIF1’, | KLF13 | 50 | 1 | 0.02 |
| | ‘BCL6’, | UNC5B | 109 | 2 | 0.018348624 |
| | ‘ZBTB16’, | HIC1 | 226 | 4 | 0.017699115 |
| | ‘MEIS1’]166 | UBC | 9403 | 139 | 0.014782516 |
| | | KLF9 | 140 | 2 | 0.014285714 |
| | | TNS1 | 850 | 12 | 0.014117647 |
| | | APOLD1 | 2453 | 34 | 0.013860579 |
| | | BTG2 | 3433 | 47 | 0.01369065 |
| | | TGIF1 | 221 | 3 | 0.013574661 |
| | | SPARC | 2584 | 34 | 0.013157895 |
| | | PITX1 | 9107 | 110 | 0.012078621 |
| | | PLEC | 1987 | 23 | 0.011575239 |
| | | GATA6 | 527 | 6 | 0.011385199 |
| | | COL6A3 | 104 | 1 | 0.009615385 |
| | | ZFP36L2 | 105 | 1 | 0.00952381 |
| | | SDC1 | 3885 | 37 | 0.00952381 |
| | | PER1 | 671255 | 6205 | 0.009243879 |
| | | PWWP2B | 221 | 2 | 0.009049774 |
| | | FAM53B | 225 | 2 | 0.008888889 |
| | | SERPINF1 | 920 | 8 | 0.008695652 |
| | | FAM129B | 93889 | 790 | 0.008414191 |
| | | SLC16A3 | 4865 | 40 | 0.008221994 |
| | | TSC22D3 | 7803 | 59 | 0.007561194 |
| | | NAGLU | 5063 | 37 | 0.00730792 |
| | | B4GALT1 | 1787 | 13 | 0.007274762 |
| | | TBX3 | 570 | 4 | 0.007017544 |
| | | MMP14 | 2594 | 18 | 0.00693909 |
| | | BCL2L1 | 9949 | 68 | 0.006834858 |
| | | BHLHE40 | 296 | 2 | 0.006756757 |
| | | ACTB | 450 | 3 | 0.006666667 |
| | | MALAT1 | 2222 | 14 | 0.00630063 |
| | | MEIS1 | 322 | 2 | 0.00621118 |
| | | NEK6 | 2626 | 16 | 0.006092917 |
| | | TEAD1 | 628464 | 3558 | 0.005661422 |
| | | SPEN | 52570 | 293 | 0.005573521 |
| | | RAI1 | 3966 | 22 | 0.005547151 |
| | | ECE1 | 2824 | 14 | 0.004957507 |
| | | KLF6 | 2304 | 11 | 0.004774306 |
| | | PVRL1 | 1924 | 9 | 0.004677755 |
| | | ETS2 | 435 | 2 | 0.004597701 |
| | | ATN1 | 32370 | 144 | 0.004448563 |
| | | COL1A1 | 1398 | 6 | 0.004291845 |
| | | IGFBP4 | 1404 | 6 | 0.004273504 |
| | | MYH9 | 1425 | 6 | 0.004210526 |
| | | DDIT4 | 484 | 2 | 0.004132231 |
| | | PTCH1 | 8270 | 34 | 0.004111245 |
| | | RBPMS | 1743 | 7 | 0.004016064 |
| UCSD_Esophagus | [‘TFCP2L1’, | EGOT | 10057 | 1 | 9.94E−05 |
| | ‘SMAD3’, | TEF | 1368 | 401 | 0.293128655 |
| | ‘ELF3’, | LYPD3 | 31 | 8 | 0.258064516 |
| | ‘GTF2I’, | CRNN | 54 | 13 | 0.240740741 |
| | ‘SREBF1’, | ALDH2 | 1265 | 116 | 0.091699605 |
| | ‘MEIS1’, | TSPAN18 | 34 | 3 | 0.088235294 |
| | ‘FOXF2’, | TPM4 | 47 | 4 | 0.085106383 |
| | ‘NR4A1’, | NEURL | 12 | 1 | 0.083333333 |
| | ‘SREBF2’, | MYEOV | 56 | 4 | 0.071428571 |
| | ‘FOXP1’, | MFAP4 | 20 | 1 | 0.05 |
| | ‘KLF4’, | ZNF217 | 102 | 5 | 0.049019608 |
| | ‘HES1’, | NKD1 | 43 | 2 | 0.046511628 |
| | ‘ZBTB16’, | TRIM29 | 72 | 3 | 0.041666667 |
| | ‘DBP’, | PPL | 991 | 41 | 0.041372351 |
| | ‘FOXA1’, | TSKU | 1912 | 77 | 0.040271967 |
| | ‘ATF4’, | BHLHE40 | 296 | 11 | 0.037162162 |
| | ‘NFE2L1’, | TACC2 | 27 | 1 | 0.037037037 |
| | ‘TGIF1’]711 | SOX7 | 81 | 3 | 0.037037037 |
| | | PKP1 | 83 | 3 | 0.036144578 |
| | | KLF5 | 348 | 12 | 0.034482759 |
| | | MIR21 | 1479 | 48 | 0.032454361 |
| | | FAT2 | 31 | 1 | 0.032258065 |
| | | RFX2 | 32 | 1 | 0.03125 |
| | | KAZ | 200 | 6 | 0.03 |
| | | PCDH1 | 34 | 1 | 0.029411765 |
| | | VSNL1 | 140 | 4 | 0.028571429 |
| | | FOXK1 | 36 | 1 | 0.027777778 |
| | | ZBTB17 | 109 | 3 | 0.027522936 |
| | | MYOF | 37 | 1 | 0.027027027 |
| | | AFAP1 | 115 | 3 | 0.026086957 |
| | | NXN | 201 | 5 | 0.024875622 |
| | | KANK1 | 41 | 1 | 0.024390244 |
| | | KRT13 | 584 | 14 | 0.023972603 |
| | | ARL4D | 42 | 1 | 0.023809524 |
| | | CDH1 | 1925 | 45 | 0.023376623 |
| | | TACC1 | 43 | 1 | 0.023255814 |
| | | SUN1 | 129 | 3 | 0.023255814 |
| | | FOXF2 | 44 | 1 | 0.022727273 |
| | | NAA20 | 45 | 1 | 0.022222222 |
| | | LASP1 | 92 | 2 | 0.02173913 |
| | | LTBP4 | 47 | 1 | 0.021276596 |
| | | SMTN | 96 | 2 | 0.020833333 |
| | | P4HB | 10369 | 215 | 0.020734883 |
| | | S1PR5 | 106 | 2 | 0.018867925 |
| | | EHD2 | 53 | 1 | 0.018867925 |
| | | FOXA1 | 544 | 10 | 0.018382353 |
| | | HS6ST1 | 111 | 2 | 0.018018018 |
| | | PGAM1 | 56 | 1 | 0.017857143 |
| | | FOXP1 | 284 | 5 | 0.017605634 |
| | | ARHGEF4 | 57 | 1 | 0.01754386 |
| UCSD_Gastric | [‘SMAD3’, | C19orf61 | 1 | 1 | 1 |
| | ‘SREBF1’, | GNA12 | 2970 | 1699 | 0.572053872 |
| | ‘HES1’, | CLDN18 | 48 | 24 | 0.5 |
| | ‘ELF3’, | HCG27 | 5 | 2 | 0.4 |
| | ‘FOXA1’, | GCNT4 | 5 | 2 | 0.4 |
| | ‘NR4A2’, | CAPN9 | 18 | 6 | 0.333333333 |
| | ‘PATZ1’, | ZKSCAN1 | 11 | 3 | 0.272727273 |
| | ‘MAZ’, | FRAT2 | 21 | 5 | 0.238095238 |
| | ‘SREBF2’, | CDH1 | 1925 | 350 | 0.181818182 |
| | ‘GTF2I’, | JAG1 | 7483 | 1354 | 0.180943472 |
| | ‘ATF4’, | GPR146 | 6 | 1 | 0.166666667 |
| | ‘TGIF1’]866 | SLC9A4 | 63 | 10 | 0.158730159 |
| | | PGA4 | 27 | 4 | 0.148148148 |
| | | PSCA | 298 | 43 | 0.144295302 |
| | | TACC1 | 43 | 6 | 0.139534884 |
| | | FOXQ1 | 59 | 8 | 0.13559322 |
| | | HRH2 | 179 | 23 | 0.12849162 |
| | | RAB40C | 9 | 1 | 0.111111111 |
| | | ZFHX3 | 84 | 9 | 0.107142857 |
| | | TFF1 | 2338 | 243 | 0.103934987 |
| | | FZD5 | 88 | 9 | 0.102272727 |
| | | ZNF217 | 102 | 10 | 0.098039216 |
| | | NEURL | 12 | 1 | 0.083333333 |
| | | MIRLET7A3 | 12 | 1 | 0.083333333 |
| | | GRB7 | 216 | 18 | 0.083333333 |
| | | CHD9 | 13 | 1 | 0.076923077 |
| | | LASP1 | 92 | 7 | 0.076086957 |
| | | SH3GL1 | 186 | 14 | 0.075268817 |
| | | RAB11B | 40 | 3 | 0.075 |
| | | TACC2 | 27 | 2 | 0.074074074 |
| | | FOXP4 | 27 | 2 | 0.074074074 |
| | | KLF6 | 2304 | 151 | 0.065538194 |
| | | PTP4A3 | 467 | 30 | 0.064239829 |
| | | EBAG9 | 169 | 10 | 0.059171598 |
| | | SEC14L1 | 18 | 1 | 0.055555556 |
| | | GATA5 | 184 | 10 | 0.054347826 |
| | | ATP1B1 | 92 | 5 | 0.054347826 |
| | | PAK4 | 149 | 8 | 0.053691275 |
| | | KCNQ1 | 2424 | 130 | 0.053630363 |
| | | MYEOV | 56 | 3 | 0.053571429 |
| | | PIM3 | 131 | 7 | 0.053435115 |
| | | TEF | 1368 | 73 | 0.053362573 |
| | | P4HB | 10369 | 548 | 0.052849841 |
| | | S100P | 253 | 13 | 0.051383399 |
| | | PPP2R1B | 80 | 4 | 0.05 |
| | | LOC100130872- | 20 | 1 | 0.05 |
| | | SPON2 | | | |
| | | DAPK1 | 990 | 49 | 0.049494949 |
| | | GATA6 | 527 | 26 | 0.049335863 |
| | | ANXA4 | 42 | 2 | 0.047619048 |
| | | PTP4A1 | 65 | 3 | 0.046153846 |
| UCSD_Left_Ventricle | [‘NFE2L1’, | C15orf52 | 1 | 1 | 1 |
| | ‘SMAD3’, | TNNT2 | 1719 | 1609 | 0.936009308 |
| | ‘RREB1’, | NKX2-5 | 1226 | 1095 | 0.89314845 |
| | ‘NR4A1’, | RBM20 | 16 | 14 | 0.875 |
| | ‘MEIS1’, | CASQ2 | 157 | 133 | 0.847133758 |
| | ‘ARID5B’, | LMOD2 | 6 | 5 | 0.833333333 |
| | ‘ZBTB16’]764 | TBX20 | 97 | 80 | 0.824742268 |
| | | MYL3 | 75 | 60 | 0.8 |
| | | PKP2 | 131 | 119 | 0.78807947 |
| | | LMNA | 23436 | 18416 | 0.785799625 |
| | | PRKAG2 | 5788 | 4453 | 0.76935038 |
| | | CMYA5 | 19 | 14 | 0.736842105 |
| | | AKAP6 | 53 | 39 | 0.735849057 |
| | | NPPB | 7829 | 5493 | 0.701622174 |
| | | FABP3 | 744 | 505 | 0.678763441 |
| | | MYOCD | 68 | 46 | 0.676470588 |
| | | MEF2A | 1446 | 914 | 0.63208852 |
| | | MEF2D | 168 | 103 | 0.613095238 |
| | | MYL2 | 230 | 140 | 0.608695652 |
| | | GATA4 | 1442 | 875 | 0.606796117 |
| | | RBM24 | 10 | 6 | 0.6 |
| | | ACTC1 | 122 | 73 | 0.598360656 |
| | | KCNH2 | 3015 | 1784 | 0.591708126 |
| | | MYH7 | 1103 | 642 | 0.582048957 |
| | | MYH6 | 1310 | 762 | 0.581679389 |
| | | PYGB | 47 | 27 | 0.574468085 |
| | | SLC8A1 | 630 | 348 | 0.552380952 |
| | | TRIM55 | 31 | 17 | 0.548387097 |
| | | MIR1-1 | 133 | 70 | 0.526315789 |
| | | KCNQ1 | 2424 | 1268 | 0.52310231 |
| | | ZNF778 | 2 | 1 | 0.5 |
| | | PPAPDC3 | 2 | 1 | 0.5 |
| | | C14orf4 | 2 | 1 | 0.5 |
| | | ADRB1 | 5293 | 2627 | 0.496315889 |
| | | NRAP | 49 | 24 | 0.489795918 |
| | | FHOD3 | 25 | 12 | 0.48 |
| | | RYR2 | 5811 | 2617 | 0.450352779 |
| | | SNTA1 | 35 | 15 | 0.428571429 |
| | | PLB1 | 1114 | 468 | 0.42010772 |
| | | ACTN2 | 63 | 26 | 0.412698413 |
| | | CKMT2 | 30 | 12 | 0.4 |
| | | AFAP1L1 | 5 | 2 | 0.4 |
| | | TPM1 | 243 | 95 | 0.390946502 |
| | | FOXK1 | 36 | 14 | 0.388888889 |
| | | CACNB2 | 80 | 31 | 0.3875 |
| | | MYPN | 16 | 6 | 0.375 |
| | | CAMK2D | 60 | 22 | 0.366666667 |
| | | NACC2 | 142 | 50 | 0.352112676 |
| | | NAV1 | 2951 | 1039 | 0.352084039 |
| | | PPP1R12B | 20 | 7 | 0.35 |
| UCSD_Lung | [‘FLI1’, | SFTA3 | 1 | 1 | 1 |
| | ‘SREBF2’, | SFTA2 | 3 | 3 | 1 |
| | ‘SREBF1’, | C8orf46 | 1 | 1 | 1 |
| | ‘RREB1’, | SFTPB | 1245 | 1165 | 0.935742972 |
| | ‘MEIS1’, | THSD4 | 11 | 7 | 0.636363636 |
| | ‘ZNF423’, | LRRC33 | 2 | 1 | 0.5 |
| | ‘TGIF1’, | ZNF444 | 6 | 2 | 0.333333333 |
| | ‘NR4A2’, | TNS3 | 9 | 3 | 0.333333333 |
| | ‘ZBTB16’, | RNF19B | 9 | 3 | 0.333333333 |
| | ‘ARID5B’, | GRTP1 | 3 | 1 | 0.333333333 |
| | ‘SMAD3’]905 | GPR116 | 15 | 5 | 0.333333333 |
| | | C3orf21 | 3 | 1 | 0.333333333 |
| | | ARHGAP23 | 3 | 1 | 0.333333333 |
| | | PPM1K | 1095 | 364 | 0.332420091 |
| | | LPCAT1 | 68 | 22 | 0.323529412 |
| | | LRRC8A | 7 | 2 | 0.285714286 |
| | | GNA15 | 7 | 2 | 0.285714286 |
| | | TMSB10 | 107 | 30 | 0.280373832 |
| | | PTBP1 | 3614 | 953 | 0.263696735 |
| | | MTSS1L | 4 | 1 | 0.25 |
| | | KIAA0247 | 4 | 1 | 0.25 |
| | | PCID2 | 1940 | 454 | 0.234020619 |
| | | ACVRL1 | 2049 | 478 | 0.233284529 |
| | | FNIP2 | 13 | 3 | 0.230769231 |
| | | PPP2R1B | 80 | 18 | 0.225 |
| | | VGLL4 | 9 | 2 | 0.222222222 |
| | | HLF | 608 | 125 | 0.205592105 |
| | | ZC3H7A | 5 | 1 | 0.2 |
| | | PTTG1IP | 855 | 171 | 0.2 |
| | | MFAP4 | 20 | 4 | 0.2 |
| | | HSP90B3P | 5 | 1 | 0.2 |
| | | CSRNP1 | 15 | 3 | 0.2 |
| | | ANXA11 | 27 | 5 | 0.185185185 |
| | | AKNA | 11 | 2 | 0.181818182 |
| | | ACO2 | 133 | 24 | 0.180451128 |
| | | EPAS1 | 789 | 141 | 0.178707224 |
| | | SPTBN1 | 2440 | 431 | 0.176639344 |
| | | MED15 | 222 | 39 | 0.175675676 |
| | | HDGF | 131 | 23 | 0.175572519 |
| | | LATS2 | 413 | 72 | 0.17433414 |
| | | KLF2 | 351 | 59 | 0.168091168 |
| | | ARHGEF17 | 12 | 2 | 0.166666667 |
| | | LAMA5 | 37 | 6 | 0.162162162 |
| | | SLC16A3 | 4865 | 777 | 0.15971223 |
| | | ENO1 | 4302 | 683 | 0.158763366 |
| | | SASH1 | 19 | 3 | 0.157894737 |
| | | MYO18A | 27 | 4 | 0.148148148 |
| | | ABLIM3 | 7 | 1 | 0.142857143 |
| | | LIMD1 | 29 | 4 | 0.137931034 |
| | | EGFR | 67027 | 9126 | 0.136154087 |
| UCSD_Ovary | [‘WT1’, | AGAP11 | 1 | 1 | 1 |
| | ‘N4A2’, | PISRT1 | 13 | 6 | 0.461538462 |
| | ‘NR4A1’, | MXRA7 | 3 | 1 | 0.333333333 |
| | ‘FOXO3’, | EGFLAM | 4 | 1 | 0.25 |
| | ‘KLF4’, | MIR202 | 9 | 2 | 0.222222222 |
| | ‘TEF’, | CHST3 | 5360 | 800 | 0.149253731 |
| | ‘SREBF1’]427 | BNC2 | 27 | 4 | 0.148148148 |
| | | GPR78 | 15 | 2 | 0.133333333 |
| | | CAPN5 | 83 | 10 | 0.120481928 |
| | | IGFBP4 | 1404 | 151 | 0.107549858 |
| | | PPP2R1B | 80 | 8 | 0.1 |
| | | ISLR | 10 | 1 | 0.1 |
| | | EDN2 | 190 | 18 | 0.094736842 |
| | | IGFBP5 | 854 | 79 | 0.092505855 |
| | | ZMYND8 | 11 | 1 | 0.090909091 |
| | | EPHX3 | 550 | 48 | 0.087272727 |
| | | GREB1 | 61 | 5 | 0.081967213 |
| | | PRKACA | 41 | 3 | 0.073170732 |
| | | WT1 | 3384 | 244 | 0.072104019 |
| | | GATA6 | 527 | 37 | 0.070208729 |
| | | SCARB1 | 2019 | 134 | 0.06636949 |
| | | GATA4 | 1442 | 88 | 0.061026352 |
| | | FOXO3 | 1586 | 88 | 0.055485498 |
| | | RGS10 | 56 | 3 | 0.053571429 |
| | | SMOC2 | 38 | 2 | 0.052631579 |
| | | BMP8A | 19 | 1 | 0.052631579 |
| | | CTDSP2 | 271 | 14 | 0.051660517 |
| | | TSHZ3 | 20 | 1 | 0.05 |
| | | MIR23B | 40 | 2 | 0.05 |
| | | KLF9 | 140 | 7 | 0.05 |
| | | HIC1 | 226 | 11 | 0.048672566 |
| | | CTDSP1 | 173 | 8 | 0.046242775 |
| | | PKNOX2 | 22 | 1 | 0.045454545 |
| | | COL16A1 | 22 | 1 | 0.045454545 |
| | | STAR | 13238 | 558 | 0.042151382 |
| | | GPX3 | 366 | 15 | 0.040983607 |
| | | ZBTB38 | 25 | 1 | 0.04 |
| | | FOSL2 | 260 | 10 | 0.038461538 |
| | | PTMA | 131 | 5 | 0.038167939 |
| | | INSR | 47446 | 1790 | 0.0377271 |
| | | EGFR | 67027 | 2498 | 0.037268563 |
| | | HDAC7 | 162 | 6 | 0.037037037 |
| | | PSMA6 | 1554 | 57 | 0.036679537 |
| | | ZNF469 | 4129 | 149 | 0.036086219 |
| | | ZMIZ1 | 201 | 7 | 0.034825871 |
| | | CDH11 | 11787 | 410 | 0.034784084 |
| | | NR1D1 | 748 | 26 | 0.034759358 |
| | | LTBP2 | 117 | 4 | 0.034188034 |
| | | PLD1 | 502 | 17 | 0.033864541 |
| | | NR2F2 | 473 | 16 | 0.033826638 |
| UCSD_Pancreas | [‘HES1, | PNLIPRP1 | 31 | 29 | 0.935483871 |
| | ‘NR5A2’, | PTF1A | 173 | 123 | 0.710982659 |
| | ‘PDX1’, | BHLHA15 | 72 | 35 | 0.486111111 |
| | ‘ELF3’, | EPN3 | 5 | 2 | 0.4 |
| | ‘NR4A2’, | ONECUT1 | 206 | 72 | 0.349514563 |
| | ‘PATZ1’, | ARHGEF10L | 3 | 1 | 0.333333333 |
| | ‘NR4A1’, | SOX13 | 44 | 13 | 0.295454545 |
| | ‘DBP’, | GNAI2 | 2970 | 826 | 0.278114478 |
| | ‘HIF1A’]399 | PDX1 | 6404 | 1629 | 0.254372267 |
| | | CDR2L | 4 | 1 | 0.25 |
| | | RPH3AL | 42 | 9 | 0.214285714 |
| | | HNF1B | 1221 | 246 | 0.201474201 |
| | | MNX1 | 282 | 50 | 0.177304965 |
| | | LAD1 | 653 | 101 | 0.15467075 |
| | | SNED1 | 199 | 30 | 0.150753769 |
| | | MRPL37 | 7 | 1 | 0.142857143 |
| | | PLA2G1B | 4467 | 575 | 0.128721737 |
| | | GPRC5C | 8 | 1 | 0.125 |
| | | INSR | 47446 | 5701 | 0.120157653 |
| | | CBX4 | 1311 | 152 | 0.115942029 |
| | | LLGL2 | 201 | 23 | 0.114427861 |
| | | SLC39A14 | 64 | 7 | 0.109375 |
| | | ATN1 | 32370 | 2977 | 0.091967871 |
| | | SLC29A1 | 415 | 38 | 0.091566265 |
| | | ZMYND8 | 11 | 1 | 0.090909091 |
| | | CDX2 | 1304 | 111 | 0.085122699 |
| | | ANP32A | 229 | 19 | 0.082969432 |
| | | RAI1 | 3966 | 286 | 0.07211296 |
| | | BCL9L | 29 | 2 | 0.068965517 |
| | | CSRNP1 | 15 | 1 | 0.066666667 |
| | | FXYD2 | 77 | 5 | 0.064935065 |
| | | IL22RA1 | 16 | 1 | 0.0625 |
| | | HES1 | 1584 | 98 | 0.061868687 |
| | | HPCAL1 | 33 | 2 | 0.060606061 |
| | | XBP1 | 1136 | 67 | 0.058978873 |
| | | ZBTB4 | 17 | 1 | 0.058823529 |
| | | LZTS2 | 17 | 1 | 0.058823529 |
| | | SOX4 | 231 | 13 | 0.056277056 |
| | | DUSP6 | 303 | 16 | 0.052805281 |
| | | TPCN1 | 96 | 5 | 0.052083333 |
| | | RAB20 | 20 | 1 | 0.05 |
| | | DAGLA | 63 | 3 | 0.047619048 |
| | | IER3 | 212 | 10 | 0.047169811 |
| | | SPRED2 | 44 | 2 | 0.045454545 |
| | | NUAK2 | 48 | 2 | 0.041666667 |
| | | SFRP5 | 148 | 6 | 0.040540541 |
| | | PAK4 | 149 | 6 | 0.040268456 |
| | | CAMKK1 | 25 | 1 | 0.04 |
| | | DUSP8 | 76 | 3 | 0.039473684 |
| | | HDGF | 131 | 5 | 0.038167939 |
| UCSD_Psoas_Muscle | [‘NR4A1’, | ZCCHC24 | 1 | 1 | 1 |
| | ‘SMAD3’, | SMTNL2 | 1 | 1 | 1 |
| | ‘ZNF423’, | LMOD3 | 1 | 1 | 1 |
| | ‘GTF2I’, | FAM193B | 1 | 1 | 1 |
| | ‘RREB1’, | FBXO32 | 488 | 478 | 0.979508197 |
| | ‘SREBF1’, | OBSCN | 46 | 44 | 0.956521739 |
| | ‘DBP’, | DYSF | 421 | 386 | 0.916864608 |
| | ‘TGIF1’, | LMOD2 | 6 | 5 | 0.833333333 |
| | ‘HES1’, | MYOD1 | 3844 | 3031 | 0.788501561 |
| | ‘NR4A2’]447 | NRAP | 49 | 37 | 0.755102041 |
| | | MEF2D | 168 | 126 | 0.75 |
| | | RBM24 | 10 | 7 | 0.7 |
| | | CAPN3 | 481 | 324 | 0.673596674 |
| | | MYOM2 | 9 | 6 | 0.666666667 |
| | | PRKAG3 | 92 | 59 | 0.641304348 |
| | | SORBS3 | 57 | 36 | 0.631578947 |
| | | TNNC2 | 13 | 8 | 0.615384615 |
| | | MIR1-1 | 133 | 81 | 0.609022556 |
| | | FOXK1 | 36 | 21 | 0.583333333 |
| | | DUSP27 | 7 | 4 | 0.571428571 |
| | | SCN4A | 839 | 473 | 0.563766389 |
| | | TMOD1 | 121 | 68 | 0.561983471 |
| | | CKM | 327 | 171 | 0.52293578 |
| | | PYGM | 160 | 83 | 0.51875 |
| | | CACNA1S | 877 | 452 | 0.515393387 |
| | | MYLK2 | 1121 | 575 | 0.51293488 |
| | | RBM20 | 16 | 8 | 0.5 |
| | | MIR365-1 | 2 | 1 | 0.5 |
| | | ASB8 | 2 | 1 | 0.5 |
| | | SYNPO2 | 33 | 14 | 0.424242424 |
| | | NFATC3 | 215 | 86 | 0.4 |
| | | PLB1 | 1114 | 419 | 0.376122083 |
| | | FABP3 | 744 | 270 | 0.362903226 |
| | | PPARGC1B | 213 | 76 | 0.356807512 |
| | | RNF122 | 3 | 1 | 0.333333333 |
| | | MRPS18A | 3 | 1 | 0.333333333 |
| | | ADSSL1 | 3 | 1 | 0.333333333 |
| | | ABLIM2 | 3 | 1 | 0.333333333 |
| | | CNBP | 6556 | 2132 | 0.325198292 |
| | | IRS1 | 2857 | 845 | 0.295764788 |
| | | PDE4DIP | 35 | 10 | 0.285714286 |
| | | FEM1A | 14 | 4 | 0.285714286 |
| | | AHNAK | 95 | 26 | 0.273684211 |
| | | MIR499 | 11 | 3 | 0.272727273 |
| | | TRPM4 | 203 | 55 | 0.270935961 |
| | | ATOH8 | 15 | 4 | 0.266666667 |
| | | SLC6A6 | 769 | 199 | 0.258777633 |
| | | SNTA1 | 35 | 9 | 0.257142857 |
| | | PDK2 | 127 | 32 | 0.251968504 |
| | | RHOBTB1 | 8 | 2 | 0.25 |
| UCSD_Right_Atrium | [‘NR4A1’, | ZCCHC24 | 1 | 1 | 1 |
| | ‘GTF2IRD1’, | C15orf52 | 1 | 1 | 1 |
| | ‘HIF1A’, | TNNT2 | 1719 | 1594 | 0.927283304 |
| | ‘MEIS1’, | NKX2-5 | 1226 | 1092 | 0.890701468 |
| | ‘SREBF2’, | RBM20 | 16 | 14 | 0.875 |
| | ‘ZNF423’, | TBX20 | 97 | 80 | 0.824742268 |
| | ‘NR4A2’, | PRKAG2 | 5788 | 4407 | 0.761402903 |
| | ‘DBP’, | LMNA | 23436 | 16098 | 0.686891961 |
| | ‘HES1’, | MEF2A | 1446 | 912 | 0.630705394 |
| | ‘FLI1’]696 | MEF2D | 168 | 103 | 0.613095238 |
| | | GATA4 | 1442 | 872 | 0.604715673 |
| | | KCNH2 | 3015 | 1774 | 0.588391376 |
| | | MYBPC3 | 829 | 481 | 0.580217129 |
| | | PYGB | 47 | 27 | 0.574468085 |
| | | GJA5 | 626 | 343 | 0.547923323 |
| | | MIR1-1 | 133 | 70 | 0.526315789 |
| | | ZNF778 | 2 | 1 | 0.5 |
| | | TMEM204 | 4 | 2 | 0.5 |
| | | MYBPHL | 2 | 1 | 0.5 |
| | | C14orf4 | 2 | 1 | 0.5 |
| | | BMP10 | 49 | 24 | 0.489795918 |
| | | SMARCD3 | 49 | 23 | 0.469387755 |
| | | PLB1 | 1114 | 469 | 0.421005386 |
| | | SNTA1 | 35 | 14 | 0.4 |
| | | AFAP1L1 | 5 | 2 | 0.4 |
| | | FOXK1 | 36 | 14 | 0.388888889 |
| | | NAV1 | 2951 | 1032 | 0.349711962 |
| | | KLF15 | 86 | 30 | 0.348837209 |
| | | NACC2 | 142 | 49 | 0.345070423 |
| | | KCNA5 | 1285 | 438 | 0.340856031 |
| | | RNF122 | 3 | 1 | 0.333333333 |
| | | KBTBD13 | 3 | 1 | 0.333333333 |
| | | ADSSL1 | 3 | 1 | 0.333333333 |
| | | ADCY6 | 142 | 47 | 0.330985915 |
| | | SPNS2 | 16 | 5 | 0.3125 |
| | | NFATC3 | 215 | 65 | 0.302325581 |
| | | DBP | 10189 | 3045 | 0.298851703 |
| | | TMOD1 | 121 | 36 | 0.297520661 |
| | | FBLN2 | 24 | 7 | 0.291666667 |
| | | ADPRHL1 | 7 | 2 | 0.285714286 |
| | | ABLIM3 | 7 | 2 | 0.285714286 |
| | | GATA6 | 527 | 148 | 0.280834915 |
| | | GRK5 | 309 | 86 | 0.278317152 |
| | | MTSS1L | 4 | 1 | 0.25 |
| | | MRPL33 | 4 | 1 | 0.25 |
| | | B4GALNT3 | 4 | 1 | 0.25 |
| | | SLC9A1 | 1428 | 352 | 0.246498599 |
| | | ADCY5 | 213 | 52 | 0.244131455 |
| | | XIRP1 | 9516 | 2307 | 0.242433796 |
| | | LDB3 | 1168 | 281 | 0.240582192 |
| UCSD_Right_Ventricle | [‘GTF2IRD1’, | TNNT2 | 1719 | 1609 | 0.936009308 |
| | ‘TEF’, | NKX2-5 | 1226 | 1095 | 0.89314845 |
| | ‘NKX2-5’, | RBM20 | 16 | 14 | 0.875 |
| | ‘BCL6’ | MYL3 | 75 | 60 | 0.8 |
| | ‘TGIF1’, | PRKAG2 | 5788 | 4453 | 0.76935038 |
| | ‘FOXO3’]277 | NPPB | 7829 | 5493 | 0.701622174 |
| | | FABP3 | 744 | 505 | 0.678763441 |
| | | MEF2D | 168 | 103 | 0.613095238 |
| | | GATA4 | 1442 | 875 | 0.606796117 |
| | | KCNH2 | 3015 | 1784 | 0.591708126 |
| | | MYH6 | 1310 | 762 | 0.581679389 |
| | | PYGB | 47 | 27 | 0.574468085 |
| | | KCNQ1 | 2424 | 1268 | 0.52310231 |
| | | HSPB7 | 41 | 21 | 0.512195122 |
| | | TMEM204 | 4 | 2 | 0.5 |
| | | C14orf4 | 2 | 1 | 0.5 |
| | | SNTA1 | 35 | 15 | 0.428571429 |
| | | MIR499 | 11 | 4 | 0.363636364 |
| | | NAV1 | 2951 | 1039 | 0.352084039 |
| | | MIR637 | 6 | 2 | 0.333333333 |
| | | C14orf180 | 3 | 1 | 0.333333333 |
| | | ADSSL1 | 3 | 1 | 0.333333333 |
| | | TRPM4 | 203 | 61 | 0.300492611 |
| | | GATA6 | 527 | 150 | 0.284619981 |
| | | ADCY5 | 213 | 55 | 0.258215962 |
| | | LDB3 | 1168 | 296 | 0.253424658 |
| | | XIRP1 | 9516 | 2387 | 0.250840689 |
| | | ZNF213 | 4 | 1 | 0.25 |
| | | MTSS1L | 4 | 1 | 0.25 |
| | | MRPL33 | 4 | 1 | 0.25 |
| | | B4GALNT3 | 4 | 1 | 0.25 |
| | | RGS3 | 112 | 26 | 0.232142857 |
| | | MYOM2 | 9 | 2 | 0.222222222 |
| | | DERL3 | 9 | 2 | 0.222222222 |
| | | FTH1 | 1097 | 230 | 0.209662716 |
| | | HAND2 | 1276 | 256 | 0.200626959 |
| | | ITGA7 | 102 | 20 | 0.196078431 |
| | | BCOR | 109 | 21 | 0.19266055 |
| | | PPARGC1B | 213 | 40 | 0.187793427 |
| | | HDAC7 | 162 | 28 | 0.172839506 |
| | | AKAP1 | 520 | 87 | 0.167307692 |
| | | RAMP1 | 335 | 56 | 0.167164179 |
| | | IRF2BP2 | 12 | 2 | 0.166666667 |
| | | ACO2 | 133 | 22 | 0.165413534 |
| | | MB | 42308 | 6716 | 0.158740664 |
| | | AHNAK | 95 | 15 | 0.157894737 |
| | | PDK2 | 127 | 20 | 0.157480315 |
| | | HDAC5 | 5139 | 805 | 0.156645262 |
| | | PTMA | 131 | 20 | 0.152671756 |
| | | LIMS2 | 27 | 4 | 0.148148148 |
| UCSD_Sigmoid_Colon | [‘FLI1’, | KIAA0247 | 4 | 3 | 0.75 |
| | ‘SMAD3’, | CDX2 | 1304 | 669 | 0.51303681 |
| | ‘SREBF1’, | MYO9B | 47 | 17 | 0.361702128 |
| | ‘ELF3’, | GCNT3 | 17 | 6 | 0.352941176 |
| | ‘NR4A1’, | SLCO2B1 | 240 | 79 | 0.329166667 |
| | ‘TEF’, | SLC9A8 | 43 | 14 | 0.325581395 |
| | ‘FOXA1’, | PIGR | 350 | 104 | 0.297142857 |
| | ‘ZNF219’, | FABP1 | 645 | 183 | 0.28372093 |
| | ‘TCF7L2’, | SLC16A5 | 19 | 5 | 0.263157895 |
| | ‘SREBF2’, | NKX2-3 | 64 | 16 | 0.25 |
| | ‘TGIF1’, | AIFM3 | 4 | 1 | 0.25 |
| | ‘ATF4’]589 | PSMG1 | 1341 | 319 | 0.237882177 |
| | | SLC43A2 | 13 | 3 | 0.230769231 |
| | | FXYD3 | 60 | 13 | 0.216666667 |
| | | ZC3H7A | 5 | 1 | 0.2 |
| | | NOXO1 | 85 | 17 | 0.2 |
| | | DENND2D | 5 | 1 | 0.2 |
| | | APOLD1 | 2453 | 477 | 0.194455768 |
| | | TCF7L2 | 1739 | 337 | 0.193789534 |
| | | SPIRE2 | 11 | 2 | 0.181818182 |
| | | MRVI1 | 45 | 8 | 0.177777778 |
| | | ARHGEF17 | 12 | 2 | 0.166666667 |
| | | SLC7A6 | 80 | 13 | 0.1625 |
| | | TJP3 | 87 | 13 | 0.149425287 |
| | | DUOX2 | 172 | 25 | 0.145348837 |
| | | SLCO4A1 | 312 | 40 | 0.128205128 |
| | | ACTN1 | 55 | 7 | 0.127272727 |
| | | KLF6 | 2304 | 292 | 0.126736111 |
| | | GPRC5C | 8 | 1 | 0.125 |
| | | FZD5 | 88 | 11 | 0.125 |
| | | ARHGAP17 | 16 | 2 | 0.125 |
| | | VDR | 4435 | 525 | 0.11837655 |
| | | NOSIP | 27 | 3 | 0.111111111 |
| | | MIR26A1 | 9 | 1 | 0.111111111 |
| | | CD79A | 45509 | 5017 | 0.11024193 |
| | | IFITM2 | 55 | 6 | 0.109090909 |
| | | CELF2 | 95 | 10 | 0.105263158 |
| | | CEACAM5 | 31340 | 3292 | 0.105041481 |
| | | IL10RA | 166 | 17 | 0.102409639 |
| | | HIC1 | 226 | 22 | 0.097345133 |
| | | DHRS3 | 65 | 6 | 0.092307692 |
| | | TNFAIP2 | 77 | 7 | 0.090909091 |
| | | PLEKHA7 | 22 | 2 | 0.090909091 |
| | | NAA20 | 45 | 4 | 0.088888889 |
| | | ZNF217 | 102 | 9 | 0.088235294 |
| | | GALNT2 | 349 | 30 | 0.085959885 |
| | | LTBP4 | 47 | 4 | 0.085106383 |
| | | PTK6 | 342 | 29 | 0.084795322 |
| | | SMTN | 96 | 8 | 0.083333333 |
| | | TINAGL1 | 744 | 59 | 0.079301075 |
| UCSD_Small_Intestine | [‘NR4A1’, | SLC5A1 | 952 | 530 | 0.556722689 |
| | ‘TCF7L2’, | ZDHHC19 | 2 | 1 | 0.5 |
| | ‘SMAD3’, | C16orf72 | 2 | 1 | 0.5 |
| | ‘SREBF1’, | CDX2 | 1304 | 602 | 0.461656442 |
| | ‘DBP’, | MYO9B | 47 | 17 | 0.361702128 |
| | ‘ELF3’, | SLCO2B1 | 240 | 75 | 0.3125 |
| | ‘ZBTB16’, | MOGAT2 | 51 | 15 | 0.294117647 |
| | ‘HES1’, | SLC16A5 | 19 | 5 | 0.263157895 |
| | ‘NR4A2’, | SLC37A1 | 8 | 2 | 0.25 |
| | ‘FLI1’, | SLC35B1 | 4 | 1 | 0.25 |
| | ‘TGIF1’]554 | KIAA0247 | 4 | 1 | 0.25 |
| | | ISX | 32 | 8 | 0.25 |
| | | NKX2-3 | 64 | 15 | 0.234375 |
| | | PSMG1 | 1341 | 312 | 0.232662192 |
| | | SLC43A2 | 13 | 2 | 0.153846154 |
| | | TJP3 | 87 | 13 | 0.149425287 |
| | | HRASLS2 | 7 | 1 | 0.142857143 |
| | | ARHGAP17 | 16 | 2 | 0.125 |
| | | KLF6 | 2304 | 278 | 0.120659722 |
| | | CD79A | 45509 | 4864 | 0.106879958 |
| | | TCF7L2 | 1739 | 179 | 0.10293272 |
| | | PMVK | 187 | 18 | 0.096256684 |
| | | DHRS3 | 65 | 6 | 0.092307692 |
| | | SPIRE2 | 11 | 1 | 0.090909091 |
| | | PLEKHA7 | 22 | 2 | 0.090909091 |
| | | VDR | 4435 | 393 | 0.088613303 |
| | | DUOX2 | 172 | 15 | 0.087209302 |
| | | ENPP6 | 12 | 1 | 0.083333333 |
| | | IL10RA | 166 | 13 | 0.078313253 |
| | | SLC13A2 | 401 | 29 | 0.072319202 |
| | | ACSL5 | 194 | 13 | 0.067010309 |
| | | GATA6 | 527 | 35 | 0.066413662 |
| | | TINAGL1 | 744 | 48 | 0.064516129 |
| | | ORMDL3 | 94 | 6 | 0.063829787 |
| | | LTBP4 | 47 | 3 | 0.063829787 |
| | | TGM2 | 1544 | 97 | 0.062823834 |
| | | CDC42EP4 | 16 | 1 | 0.0625 |
| | | P4HB | 10369 | 629 | 0.060661587 |
| | | TRIM8 | 33 | 2 | 0.060606061 |
| | | COTL1 | 4184 | 249 | 0.059512428 |
| | | XPNPEP1 | 323 | 18 | 0.055727554 |
| | | SLC9A1 | 1428 | 77 | 0.053921569 |
| | | RAB20 | 20 | 1 | 0.05 |
| | | MGAT3 | 160 | 8 | 0.05 |
| | | APOLD1 | 2453 | 117 | 0.047696698 |
| | | TSPAN15 | 21 | 1 | 0.047619048 |
| | | ANPEP | 7254 | 337 | 0.046457127 |
| | | CXCR6 | 353 | 16 | 0.045325779 |
| | | LASP1 | 92 | 4 | 0.043478261 |
| | | NUDT16L1 | 24 | 1 | 0.041666667 |
| UCSD_Spleen | [‘WT1’, | ARHGAP23 | 3 | 1 | 0.333333333 |
| | ‘NFE2L1’, | RNP19B | 9 | 2 | 0.222222222 |
| | ‘SMAD3’, | ZC3H7A | 5 | 1 | 0.2 |
| | ‘TGIF1’, | MADCAM1 | 322 | 46 | 0.142857143 |
| | ‘FLI1’, | NKX2-3 | 64 | 9 | 0.140625 |
| | ‘SREBF1’, | RASA3 | 23 | 3 | 0.130434783 |
| | ‘DBP’, | SPNS2 | 16 | 2 | 0.125 |
| | ‘ZNF423’]545 | CXCR5 | 600 | 71 | 0.118333333 |
| | | ABHD2 | 78 | 8 | 0.102564103 |
| | | MFAP4 | 20 | 2 | 0.1 |
| | | C1orf38 | 10 | 1 | 0.1 |
| | | ISG20 | 13861 | 1259 | 0.090830387 |
| | | SPI1 | 2118 | 179 | 0.084513692 |
| | | IL4R | 6442 | 531 | 0.082427817 |
| | | LBR | 18340 | 1465 | 0.079880044 |
| | | ST3GAL2 | 13 | 1 | 0.076923077 |
| | | IL34 | 53 | 4 | 0.075471698 |
| | | MYO18A | 27 | 2 | 0.074074074 |
| | | CHI3L2 | 29 | 2 | 0.068965517 |
| | | NLRC5 | 44 | 3 | 0.068181818 |
| | | PLCG2 | 30 | 2 | 0.066666667 |
| | | MFNG | 30 | 2 | 0.066666667 |
| | | APOL2 | 15 | 1 | 0.066666667 |
| | | TK2 | 211 | 14 | 0.066350711 |
| | | SWAP70 | 76 | 5 | 0.065789474 |
| | | LAPTM5 | 31 | 2 | 0.064516129 |
| | | CCR7 | 2514 | 159 | 0.063245823 |
| | | CDC42EP4 | 16 | 1 | 0.0625 |
| | | CDC42EP2 | 16 | 1 | 0.0625 |
| | | ARHGAP17 | 16 | 1 | 0.0625 |
| | | ACSS1 | 16 | 1 | 0.0625 |
| | | SLC9A5 | 34 | 2 | 0.058823529 |
| | | PDLIM1 | 51 | 3 | 0.058823529 |
| | | JAG1 | 7483 | 425 | 0.056795403 |
| | | CSF1 | 25327 | 1345 | 0.053105382 |
| | | TNFAIP2 | 77 | 4 | 0.051948052 |
| | | COTL1 | 4184 | 212 | 0.050669216 |
| | | SIGLEC9 | 61 | 3 | 0.049180328 |
| | | SEMA6B | 350 | 17 | 0.048571429 |
| | | OAF | 129 | 6 | 0.046511628 |
| | | LYL1 | 65 | 3 | 0.046153846 |
| | | RELT | 22 | 1 | 0.045454545 |
| | | SLC16A6 | 23 | 1 | 0.043478261 |
| | | MIR199A1 | 46 | 2 | 0.043478261 |
| | | CMIP | 23 | 1 | 0.043478261 |
| | | MYO9B | 47 | 2 | 0.042553191 |
| | | CD79A | 45509 | 1826 | 0.040123932 |
| | | KLF13 | 50 | 2 | 0.04 |
| | | ITGB2 | 22607 | 893 | 0.03950104 |
| | | ANKRD13A | 26 | 1 | 0.038461538 |
| UCSD_Thymus | [‘SMAD3’, | CCR9 | 366 | 71 | 0.193989071 |
| | ‘RREB1’, | TCF7 | 343 | 55 | 0.160349854 |
| | ‘ZBTB16’, | TMSB10 | 107 | 16 | 0.14953271 |
| | ‘BACH2’ | CD247 | 429 | 63 | 0.146853147 |
| | ‘CTCF’, | STK17B | 42 | 6 | 0.142857143 |
| | ‘SP3’, | LCK | 3367 | 470 | 0.13959014 |
| | ‘FLI1’]376 | CD3D | 332 | 46 | 0.138554217 |
| | | CD3E | 398 | 53 | 0.133165829 |
| | | CD6 | 407 | 51 | 0.125307125 |
| | | SATB1 | 227 | 27 | 0.118942731 |
| | | LCP2 | 495 | 48 | 0.096969697 |
| | | CD7 | 2216 | 198 | 0.089350181 |
| | | HDAC7 | 162 | 14 | 0.086419753 |
| | | KLF13 | 50 | 4 | 0.08 |
| | | IKZF1 | 1278 | 99 | 0.077464789 |
| | | ISG20 | 13861 | 981 | 0.070774114 |
| | | DNTT | 5014 | 334 | 0.066613482 |
| | | ZBTB16 | 512 | 34 | 0.06640625 |
| | | CD4 | 124625 | 8177 | 0.065612839 |
| | | CD2 | 16582 | 1070 | 0.064527801 |
| | | HIST1H2AC | 147 | 9 | 0.06122449 |
| | | CD8A | 118848 | 6689 | 0.056281974 |
| | | ITPKB | 54 | 3 | 0.055555556 |
| | | ZC3HAV1 | 2531 | 136 | 0.053733702 |
| | | NPATC3 | 215 | 11 | 0.051162791 |
| | | PFN1 | 261 | 13 | 0.049808429 |
| | | CD28 | 9013 | 429 | 0.047597914 |
| | | SMARCE1 | 65 | 3 | 0.046153846 |
| | | MXD4 | 47 | 2 | 0.042553191 |
| | | PRKCQ | 404 | 17 | 0.042079208 |
| | | MEF2D | 168 | 7 | 0.041666667 |
| | | HIVEP2 | 100 | 4 | 0.04 |
| | | CCR7 | 2514 | 98 | 0.038981702 |
| | | DAD1 | 133 | 5 | 0.037593985 |
| | | GNB1L | 55 | 2 | 0.036363636 |
| | | CD99 | 1419 | 51 | 0.035940803 |
| | | RANBP3 | 30 | 1 | 0.033333333 |
| | | LAPTM5 | 31 | 1 | 0.032258065 |
| | | CXCR5 | 600 | 18 | 0.03 |
| | | C21orf33 | 1434 | 42 | 0.029288703 |
| | | NFATC1 | 3400 | 96 | 0.028235294 |
| | | IFNAR2 | 2107 | 55 | 0.026103465 |
| | | FMNL1 | 43 | 1 | 0.023255814 |
| | | ETS1 | 1684 | 38 | 0.022565321 |
| | | PLCG1 | 577 | 13 | 0.022530329 |
| | | ARL4C | 3420 | 76 | 0.022222222 |
| | | SLAMF1 | 1911 | 42 | 0.021978022 |
| | | CELF2 | 95 | 2 | 0.021052632 |
| | | TARP | 545 | 11 | 0.020183486 |
| | | CD38 | 8274 | 166 | 0.020062847 |

1. A method of identifying the core regulatory circuitry of a cell or tissue, comprising:
a) identifying a group of transcription factor encoding genes in a cell or tissue which are associated with a super-enhancer;
b) determining which transcription factor encoding genes identified in a) comprise autoregulated transcription factor encoding genes, wherein a transcription factor encoding gene identified in a) comprises an autoregulated transcription factor encoding gene if the transcription factor encoded by the transcription factor encoding gene is predicted to bind to the super-enhancer associated with the transcription factor encoding gene;
c) identifying the core regulatory circuitry of the cell or tissue, wherein the core regulatory circuitry of the cell or tissue comprises autoregulated transcription factor encoding genes identified in b) which form an interconnected autoregulatory loop, wherein the autoregulated transcription factor encoding genes identified in b) form an interconnected autoregulatory loop if each transcription factor encoded by an autoregulated transcription factor encoding gene identified in b) is predicted to bind to the super-enhancer associated with each of the other autoregulated transcription factor encoding genes identified in b).

2. The method of claim 1, wherein the core regulatory circuitry comprises the autoregulated transcription factors forming the interconnected autoregulatory loop, the transcription factors encoded by the autoregulated transcription factor encoding genes, a super-enhancers associated with the autoregulated transcription factor encoding genes, or a component of the super-enhancer.

3. The method of claim 1, further comprising d) determining at least one target of at least one transcription factor encoded by at least one autoregulated transcription factor encoding gene.

4. (canceled)

5. The method of claim 1, wherein the transcription factor encoded by the transcription factor encoding gene is predicted to bind to the super-enhancer associated with transcription factor encoding gene if the super-enhancer associated with the transcription factor encoding gene comprises at least one DNA sequence motif predicted for the transcription factor encoded by the transcription factor encoding gene.

6. The method of claim 1, wherein each transcription factor encoded by the autoregulated transcription factor encoding gene is predicted to bind to the super-enhancer associated with each of the other autoregulated transcription factor encoding genes if the super-enhancers associated with each of the other autoregulated transcription factor encoding genes comprise at least one DNA sequence motif predicted for each of the transcription factors encoded by each of the other autoregulated transcription factor encoding genes.

7. The method of claim 5, wherein the at least one DNA sequence motif is located between 500 bp upstream and 500 bp downstream of the super-enhancer associated with the transcription factor encoding gene.

8. (canceled)

9. (canceled)

10. A method of identifying the cell identity program of a cell or tissue, comprising
a) identifying the core regulatory circuitry of a cell or tissue of interest according to the method of claim 1, wherein the core regulatory circuitry of the cell or tissue of interest comprises at least one autoregulated transcription factor encoding gene associated with a super-enhancer in the cell or tissue of interest, at least one transcription factor encoded by the at least one autoregulated transcription factor encoding gene, at least one super-enhancer associated with the at least one autoregulated transcription factor encoding gene, and optionally at least one component of the super-enhancer; and
b) identifying the cell identity program of the cell or tissue, wherein the cell identity program of the cell or tissue comprises the core regulatory circuitry identified in a) and at least one target of the at least one transcription factor encoded by the at least one autoregulated transcription factor encoding gene in the core regulatory circuitry.

11. The method of claim 10, wherein the at least one target comprises a gene comprising at least one enhancer element predicted to be bound by the at least one transcription factor.

12. The method of claim 10, wherein the at least one enhancer element predicted to be bound by the at least one transcription factor comprises a DNA sequence motif associated with a super-enhancer.

13.-37. (canceled)

38. A method of identifying a candidate modulator of at least one component of the core regulatory circuitry of a cell or tissue or of at least one component of the cell identity program of a cell or tissue, comprising:
a) contacting a cell or tissue with a test agent; and
b) assessing the ability of the test agent to modulate at least one component of the core regulatory circuitry of the cell or tissue or at least one component of the cell identity program of a cell or tissue, wherein the test agent is identified as a candidate modulator of the at least one component of the core regulatory circuitry of the cell or tissue or of the at least one component of the cell identity program of a cell or tissue if the at least one component of the core regulatory circuitry or the at least one component of the cell identity program of a cell or tissue is activated or inhibited in the presence of the test agent.

39. The method of claim 38, wherein the at least one component of the core regulatory circuitry of the cell or tissue comprises a reprogramming factor or a cell identity gene.

40. The method of claim 38, wherein the at least one component of the core regulatory circuitry of the cell or tissue comprises a disease-associated variant.

41. A method of reprogramming a cell comprising contacting the cell with the candidate modulator identified according to the method of claim 38.

42. The method of claim 41, wherein at least one component of the core regulatory circuitry of the cell comprises a disease-associated variant.

43.-49. (canceled)

50. A method of identifying a target for drug discovery comprising identifying a variation in at least one component of the core regulatory circuitry of a cell or tissue that is more prevalent in subjects suffering from a disease than in healthy subjects or identifying a variation in at least one component of the cell identity program of a cell or tissue that is more prevalent in subjects suffering from a disease than in healthy subjects, wherein the at least one component of the core regulatory circuitry of the cell or tissue or the least one component of the cell identity program of a cell or tissue that is more prevalent in subjects suffering from a disease than in healthy subjects comprises a disease-associated variant, and wherein the disease-associated variant is a target for drug discovery.

51.-57. (canceled)
